(19) United States
(12) Patent Application Publication
(10) Pub. No.: US 2012/0309773 A1
(43) Pub. Date: Dec. 6, 2012

(54) HETEROCYCLIC COMPOUNDS AS JANUS KINASE INHIBITORS

(76) Inventors: Yarlagadda S. Babu, Birmingham, AL (US); Pravin L. Kotian, Birmingham, AL (US)

(21) Appl. No.: 13/519,015

(22) PCT Filed: Dec. 22, 2010
(86) PCT No.: PCT/US10/61912

§ 371 Date: Aug. 21, 2012

Related U.S. Application Data
(60) Provisional application No. 61/289,978, filed on Dec. 23, 2009.
(60) Provisional application No. 61/289,975, filed on Dec. 23, 2009.

(51) Int. Cl. A61K 31/519; A61P 35/00; A61P 37/06; C07D 491/048

(52) U.S. Cl. 514/260.1; 544/278

(57) ABSTRACT

The invention provides compounds of formula I:

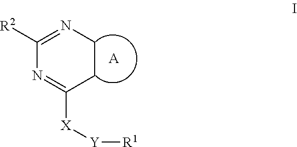

or a salt thereof as described herein. The invention also provides pharmaceutical compositions comprising a compound of formula I, processes for preparing compounds of formula I, intermediates useful for preparing compounds of formula I, and therapeutic methods for suppressing an immune response or treating cancer or a hematologic malignancy using compounds of formula I.

### CROSS-REFERENCE TO RELATED APPLICATIONS

This patent application claims the benefit of priority of U.S. application Ser. No. 61/289,978, filed Dec. 23, 2009 and U.S. application Ser. No. 61/289,975, filed Dec. 23, 2009 which applications are herein incorporated by reference.

### BACKGROUND OF THE INVENTION

Janus kinase 3 (JAK3) is a cytoplasmic protein tyrosine kinase associated with the common gamma chain (γc), which is an integral component of various cytokine receptors (Elizabeth Kudlacz et al., American Journal of Transplantation, 2004, 4, 51-57).

While effective in the prevention of transplant rejection, commonly used immunosuppressants, such as calcineurin inhibitors, possess a number of significant dose-limiting toxicities, thereby prompting a search for agents with novel mechanisms of action. The inhibition of JAK3 represents an attractive strategy for immunosuppression based upon its limited tissue distribution, lack of constitutive activation and the evidence for its role in immune cell function. JAK3 is a viable target for immunosuppression and transplant rejection. JAK3 specific inhibitors may also be useful for treatment of hematologic and other malignancies that involve pathologic JAK activation.

Currently, there is a need for compounds, compositions and methods that are useful for treating diseases and conditions associated with pathologic JAK activation.

### SUMMARY OF THE INVENTION

In one embodiment, the invention provides a compound of the invention which is a compound of formula I:

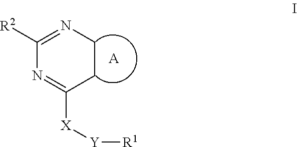

wherein:

A is furan optionally substituted with one or more (e.g. 1 or 2) R³ groups;

X is NH, O, S or absent;

Y is heteroaryl or aryl, wherein heteroaryl is linked to X by a carbon atom when X is NH, O or S and wherein any heteroaryl or aryl of Y may be optionally substituted with one or more (e.g. 1, 2, 3, 4 or 5) Rₐ groups;

R¹ is —C(O)NR_{g}Rₕ, —NRᵢC(O)NR_{g}Rₕ, —CHO, —C(O)Rⱼ, —CO₂H, —C(O)ORⱼ, —NRᵢS(O)₂NR_{g}Rₕ, —NRᵢC(O)Rⱼ, —NRᵢS(O)₂Rⱼ, —C(O)C(O)Rⱼ, —C(O)NRᵢS(O)₂Rⱼ, —C(O)NRᵢCHO, —C(O)NRᵢC(O)Rⱼ, —C≡CH, —C≡CRⱼ, —C(S)NR_{g}Rₕ, —C(═NRᵢ)NR_{g}Rₕ, (C₁-C₆)alkyl, (C₃-C₆)cycloalkyl, heterocycle, heteroaryl, aryl or is absent, wherein any alkyl, cycloalkyl, heterocycle, heteroaryl or aryl of R¹ may be optionally substituted with one or more (e.g. 1, 2 or 3) R_{z} groups;

R² is heteroaryl, —NR⁶R⁷, —OR⁸, SR⁸ or CHR⁹R¹⁰, wherein any heteroaryl of R² may be optionally substituted with one or more (e.g. 1, 2 or 3) R¹¹ groups;

each R³ is independently halo, (C₁-C₆)alkyl, (C₂-C₆)alkenyl, (C₂-C₆)alkynyl, (C₃-C₆)cycloalkyl, —ORₐ₂, —OC(O)R_{b2}, —OC(O)NR_{c2}R_{d2}, —SRₐ₂, —S(O)₂OH, —S(O)R_{b2}, —S(O)₂R_{b2}, —S(O)₂NR_{c2}R_{d2}, —NR_{c2}R_{d2}, —NRₑ₂C(O)R_{b2}, —NRₑ₂C(O)NR_{c2}R_{d2}, NRₑ₂S(O)₂R_{b2}, —NRₑ₂S(O)₂NR_{c2}R_{d2}, NO₂, —C(O)Rₐ₂, —C(O)ORₐ₂ or —C(O)NR_{c2}R_{d2};

R⁶ is selected from (C₁-C₆)alkyl, (C₂-C₆)alkenyl, (C₂-C₆)alkynyl, (C₃-C₆)cycloalkyl, heteroaryl, heterocycle and aryl, and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino or thiomorpholino of R⁶ and R⁷ may be optionally substituted with one or more (e.g. 1, 2 or 3) R¹¹ groups;

each R⁸ is independently selected from (C₁-C₆)alkyl, (C₂-C₆)alkenyl, (C₂-C₆)alkynyl, (C₃-C₆)cycloalkyl, heteroaryl and aryl, wherein any alkyl, alkenyl, alkynyl, cycloalkyl, heteroaryl or aryl of R⁸ may be optionally substituted with one or more (e.g. 1, 2 or 3) R¹¹ groups;

R⁹ is selected from (C₁-C₆)alkyl, (C₂-C₆)alkenyl, (C₂-C₆)alkynyl, (C₃-C₆)cycloalkyl, heteroaryl, heterocycle and aryl, and R¹⁰ is selected from H and (C₁-C₆)alkyl; or R⁹ and R¹⁰ together with the carbon to which they are attached form a (C₃-C₇)cycloalkyl, pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of R⁹ and R¹⁰ may be optionally substituted with one or more (e.g. 1, 2 or 3) R¹¹ groups;

each R¹¹ is independently selected from (C₁-C₆)alkyl, (C₂-C₆)alkenyl, (C₂-C₆)alkynyl, (C₃-C₆)cycloalkyl, —ORₘ, —NRₜCORₙ, NRₒRₚ, heteroaryl and aryl, wherein alkyl, alkenyl, alkynyl, cycloalkyl, heteroaryl or aryl may be optionally substituted with one or more (e.g. 1, 2, 3, 4 or 5) groups selected from halo, R_{q}, OH, CN, —OR_{q}, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, —CHO, —C(O)R_{q}, —C(O)OH, —C(O)OR_{q} and —C(O)NRᵣRₛ;

each Rₐ is independently selected from (C₁-C₆)alkyl, (C₂-C₆)alkenyl, (C₂-C₆)alkynyl, (C₃-C₆)cycloalkyl, halo, CN, —OR_{f}, —OC(O)R_{b}, —OC(O)NR_{c}R_{d}, —SR_{f}, —S(O)R_{b}, —S(O)₂OH, —S(O)₂R_{b}, —S(O)₂NR_{c}R_{d}, —NR_{c}R_{d}, —NRₑCOR_{b}, —NRₑCO₂R_{b}, —NRₑCONR_{c}R_{d}, —NRₑS(O)₂R_{b}, —NRₑS(O)₂NR_{c}R_{d}, NO₂, —C(O)R_{f}, —C(O)OR_{f} and —C(O)NR_{c}R_{d};

each R_{b} is independently (C₁-C₆)alkyl, (C₂-C₆)alkenyl, (C₂-C₆)alkynyl, (C₃-C₆)cycloalkyl, heterocycle, heteroaryl or aryl;

R_{c} and R_{d} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c} and R_{d} together with the nitrogen to which they are attached form a pyrrolidino, piperidino, piperazino, azetidino, morpholino, or thiomorpholino;

each Rₑ is independently H, (C₁-C₆)alkyl, (C₂-C₆)alkenyl, (C₂-C₆) alkynyl or (C₃-C₆)cycloalkyl;

each R_{f} is independently H, (C₁-C₆)alkyl, (C₂-C₆)alkenyl, (C₂-C₆)alkynyl, (C₃-C₆)cycloalkyl, heterocycle, heteroaryl or aryl;

R_{g} and Rₕ are each independently selected from H, (C₁-C₈)alkyl, (C₂-C₈)alkenyl, (C₂-C₈)alkynyl, (C₃-C₈)cycloalkyl, heterocycle, heteroaryl and aryl, wherein any aryl or heteroaryl of R_{g} or Rₕ may be optionally substituted with one or more (e.g. 1, 2, 3, 4 or 5) Rₖ groups and wherein any alkyl, alkenyl, alkynyl, cycloalkyl or heterocycle of R_{g} or Rₕ may be optionally substituted with one or more (e.g. 1, 2, 3, 4 or 5) oxo (C═O) or Rₖ groups; or R_{g} and Rₕ together with the nitrogen to which they are attached form a pyrrolidino, piperidino, piperazino, azetidino, morpholino, or thiomorpholino wherein any pyrrolidino, piperidino, piperazino, azetidino, morpholino or thiomorpholino of R_{g} and Rₕ may be optionally substituted with one or more (e.g. 1, 2, 3, 4 or 5) Rₖ or oxo groups;

each Rᵢ is independently H, (C₁-C₆)alkyl, (C₂-C₆)alkenyl, (C₂-C₆)alkynyl or (C₃-C₆)cycloalkyl;

each Rⱼ is independently selected from (C₁-C₆)alkyl, (C₂-C₆)alkenyl, (C₂-C₆)alkynyl, (C₃-C₆)cycloalkyl, heterocycle, heteroaryl and aryl, wherein any aryl or heteroaryl of Rⱼ may be optionally substituted with one or more (e.g. 1, 2, 3, 4 or 5) Rₖ groups and wherein any alkyl, alkenyl, alkynyl, cycloalkyl or heterocycle of Rⱼ may be optionally substituted with one or more (e.g. 1, 2, 3, 4 or 5) oxo (C═O) or Rₖ groups;

each Rₖ is independently selected from halo, R_{y}, CN, OH, —OR_{y}, —OC(O)R_{y}, —OC(O)NRᵥR_{w}, SH, —SR_{y}, —S(O)R_{y}, —S(O)₂OH, —S(O)₂R_{y}, —S(O)₂NRᵥR_{w}, —NRₓCOR_{y}, —NRₓCO₂R_{y}, —NRₓCONRᵥR_{w}, —NRₓS(O)₂R_{y}, —NRₓS(O)₂NRᵥR_{w}, NO₂, —C(O)Rᵤ, —C(O)ORᵤ and —C(O)NRᵥR_{w};

each Rₘ is independently H, (C₁-C₆)alkyl, (C₂-C₆)alkenyl, (C₂-C₆)alkynyl, (C₃-C₆)cycloalkyl, heterocycle, heteroaryl or aryl;

each Rₙ is independently (C₁-C₆)alkyl, (C₂-C₆)alkenyl, (C₂-C₆)alkynyl, (C₃-C₆)cycloalkyl, heterocycle, heteroaryl or aryl;

Rₒ and Rₚ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rₒ and Rₚ together with the nitrogen to which they are attached form a pyrrolidino, piperidino, piperazino, azetidino, morpholino, or thiomorpholino;

each R_{q} is independently (C₁-C₆)alkyl, (C₂-C₆)alkenyl, (C₂-C₆)alkynyl, (C₃-C₆)cycloalkyl, heterocycle, heteroaryl or aryl;

R^{r} and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino;

each Rₜ is independently H, (C₁-C₆)alkyl, (C₂-C₆)alkenyl, (C₂-C₆) alkynyl or (C₃-C₆)cycloalkyl;

each Rᵤ is independently H, (C₁-C₆)alkyl, (C₂-C₆)alkenyl, (C₂-C₆)alkynyl, (C₃-C₆)cycloalkyl, heterocycle, heteroaryl or aryl;

Rᵥ and R_{w} are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵥ and R_{w} together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of Rᵥ and R_{w} may be optionally substituted with one or more (e.g. 1 or 2) groups independently selected from CH₂OH, OH, NH₂ and CONH₂;

each Rₓ is independently H, (C₁-C₆)alkyl, (C₂-C₆)alkenyl, (C₂-C₆) alkynyl or (C₃-C₆)cycloalkyl;

each R_{y} is independently (C₁-C₆)alkyl, (C₂-C₆)alkenyl, (C₂-C₆)alkynyl, (C₃-C₆)cycloalkyl, heterocycle, heteroaryl or aryl, wherein any alkyl, alkenyl, alkynyl, cycloalkyl, heterocycle, heteroaryl or aryl of R_{y} may be optionally substituted with one or more (e.g. 1 or 2) groups selected from ORᵤ and NRᵥR_{w};

each R_{z} is independently halo, heteroaryl, (C₁-C₆)alkyl, CN, —O(C₁-C₆)alkyl, NO₂, —C(O)OH, —(C₁-C₆)alkylNH₂, —(C₁-C₆)alkylOH, —NHC(O)(C₁-C₆)alkyl or —NHC(O)(C₁-C₆)alkylCN, wherein heteroaryl is optionally substituted with —(C₁-C₆)alkylNH₂ or —(C₁-C₆)alkylOH;

each Rₐ₂ is independently H, (C₁-C₆)alkyl, (C₂-C₆)alkenyl, (C₂-C₆)alkynyl, (C₃-C₆)cycloalkyl, heterocycle, heteroaryl or aryl;

each R_{b2} is independently (C₁-C₆)alkyl, (C₂-C₆)alkenyl, (C₂-C₆)alkynyl, (C₃-C₆)cycloalkyl, heterocycle, heteroaryl or aryl;

R_{c2} and R_{d2} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c2} and R_{d2} together with the nitrogen to which they are attached form a pyrrolidino, piperidino, piperazino, azetidino, morpholino, or thiomorpholino; and

each Rₑ₂ is independently H, (C₁-C₆)alkyl, (C₂-C₆)alkenyl, (C₂-C₆) alkynyl or (C₃-C₆)cycloalkyl;

or a salt thereof.

The invention also provides a pharmaceutical composition comprising a compound of formula I or a pharmaceutically acceptable salt thereof, and a pharmaceutically acceptable diluent or carrier.

The invention also provides method for treating a disease or condition associated with pathologic JAK activation (e.g. a cancer, a hematologic malignancy or other malignancy) in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic treatment of a disease or condition associated with pathologic JAK activation (e.g. a cancer, a hematologic malignancy or other malignancy).

The invention also provides a compound of formula I, or a pharmaceutically acceptable salt thereof for use in medical therapy (e.g. for use in treating a disease or condition associated with pathologic JAK activation such as cancer, a hematologic malignancy or other malignancy).

The invention also provides a compound of formula I or a pharmaceutically acceptable salt thereof for the manufacture of a medicament for the treatment of a disease or condition associated with pathologic JAK activation (e.g. a cancer, a hematologic malignancy or other malignancy) in a mammal (e.g. a human).

The invention also provides a method for suppressing an immune response in a mammal (e.g. a human), comprising administering a compound of formula I, or a pharmaceutically acceptable salt thereof, to the mammal.

The invention also provides a compound of formula I, or a pharmaceutically acceptable salt thereof, for use in the prophylactic or therapeutic suppression of an immune response.

The invention also provides the use of a compound of formula I, or a pharmaceutically acceptable salt thereof for the manufacture of a medicament for suppressing an immune response in a mammal (e.g. a human).

The invention also provides novel processes and novel intermediates disclosed herein that are useful for preparing compounds of formula I or salts thereof, for example, those described in Schemes 1-19.

### DETAILED DESCRIPTION OF THE INVENTION

### Definitions

The term “alkyl” as used herein refers to alkyl groups having from 1 to 10 carbon atoms which are straight or branched monovalent groups. This term is exemplified by groups such as methyl, ethyl, n-propyl, iso-propyl, n-butyl, t-butyl, isobutyl, n-pentyl, neopentyl, and n-hexyl, and the like

The terms “alkenyl” or “alkene” as used herein refers to an alkenyl group having from 2 to 10 carbon atoms which are straight or branched monovalent groups and having at least one double bond. Such groups are exemplified by vinykethen-1-yl), allyl, 1-propenyl, 2-propenyl(allyl), 1-methylethen-1-yl, 1-buten-1-yl, 2-buten-1-yl, 3-buten-1-yl, 1-methyl-1-propen-1-yl, 2-methyl-1-propen-1-yl, 1-methyl-2-propen-1-yl, and 2-methyl-2-propen-1-yl, preferably 1-methyl-2-propen-1-yl, 3,5-hexadien-1-yl and the like.

The term “alkynyl” or “alkyne” as used herein refers to an alkynyl group having from 2-10 carbon atoms which are straight or branched monovalent groups and having at least one triple bond. Such groups are exemplified by, but not limited to ethyn-1-yl, propyn-1-yl, propyn-2-yl, 1-methylprop-2-yn-1-yl, butyn-1-yl, butyn-2-yl, butyn-3-yl, 3,5-hexadiyn-1-yl and the like.

The term “halo” as used herein refers to fluoro, chloro, bromo and iodo. The term “cycloalkyl” as used herein refers to a saturated or partially unsaturated cyclic hydrocarbon ring systems, such as those containing 1 to 3 rings and 3 to 8 carbons per ring wherein multiple ring cycloalkyls can have fused, bridging and spiro bonds to one another. Exemplary groups include but are not limited to cyclopropyl, cyclobutyl, cyclopentyl, cyclohexyl, cycloheptyl, cyclooctyl, cyclobutenyl, cyclohexenyl, cyclooctadienyl, decahydronaphthalene and spiro[4.5]decane.

The term “aryl” as used herein refers to an aromatic cyclic group of from 6 to 14 carbon atoms having a single ring (e.g. phenyl) or multiple condensed rings (e.g. naphthyl or anthryl) wherein the condensed rings may be aromatic, saturated or partially saturated provided that at least one of the condensed rings is aromatic. Such multiple condensed rings may be optionally substituted with one or two oxo groups on the unsaturated or partially unsaturated ring portions of the multiple condensed ring. Exemplary aryls include, but are not limited to phenyl, indanyl naphthyl, 1,2-dihydronaphthyl and 1,2,3,4-tetrahydronaphthyl.

The term “heteroaryl” as used herein refers to a single aromatic ring of from about 1 to 6 carbon atoms and about 1-4 heteroatoms selected from the group consisting of oxygen, nitrogen and sulfur in the rings. The sulfur and nitrogen atoms may also be present in their oxidized forms. Such rings include but are not limited to pyridyl, pyrimidinyl, oxazolyl or furyl. The term heteroaryl also includes multiple condensed ring systems wherein a heteroaryl group (as defined above) can be fused with another heteroaryl (e.g. naphthyridinyl), a cycloalkyl (e.g. 5,6,7,8-tetrahydroquinolyl), an aryl (e.g. indazolyl) or a heterocycle (1,2,3,4-tetrahydronaphthyridine) to form a multiple condensed ring. Such multiple condensed rings may be optionally substituted with one or two oxo groups on the cycloalkyl or heterocycle portions of the condensed ring. Exemplary heteroaryls include but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and 4,5,6,7-tetrahydroindolyl.

The term “heterocycle” or “heterocyclic” or “heterocycloalkyl” as used herein refers to a single saturated or partially unsaturated ring (e.g. 3, 4, 5, 6, 7 or 8-membered ring) from about 1 to 7 carbon atoms and from about 1 to 3 heteroatoms selected from the group consisting of oxygen, nitrogen and sulfur in the rings. The sulfur and nitrogen atoms may also be present in their oxidized forms. Such rings include but are not limited to azetidinyl, tetrahydrofuranyl or piperidinyl. The term heterocycle also includes multiple condensed ring systems wherein a heterocycle group (as defined above) can be fused with another heterocycle (e.g. decahydronapthyridinyl), a cycloalkyl (e.g. decahydroquinolyl) or an aryl (e.g. 1,2,3,4-tetrahydroisoquinolyl) to form a multiple condensed ring. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, benzoxazinyl and dihydrooxazolyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts, including pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Specific values listed below for radicals, substituents, and ranges, are for illustration only; they do not exclude other defined values or other values within defined ranges for the radicals and substituents. The specific values listed below are specific values for compounds of formula I as well as compounds of formula IIa, IIb, IIc, IId, IIe, IIf and IIg.

A specific group of compounds of formula I are compounds of formula IIa:

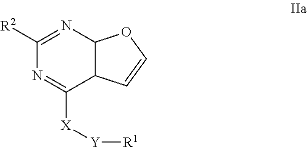

or a salt thereof.

A specific group of compounds of formula I are compounds of formula IIb:

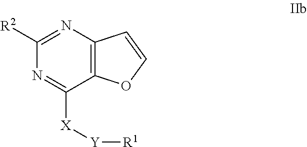

or a salt thereof.

A specific group of compounds of formula I are compounds of formula IIc:

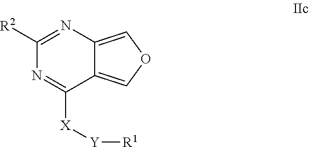

or a salt thereof.

A specific group of compounds of formula I are compounds of formula IIb:

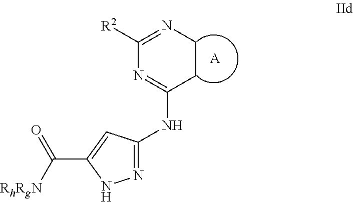

or a salt thereof.

A specific group of compounds of formula I are compounds of formula IIb:

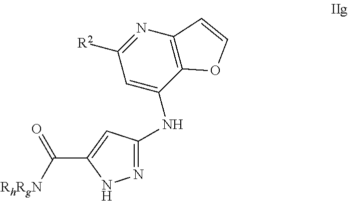

or a salt thereof.

A specific group of compounds of formula I are compounds of formula IIb:

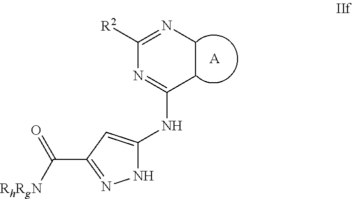

or a salt thereof.

A specific group of compounds of formula I are compounds of formula IIb:

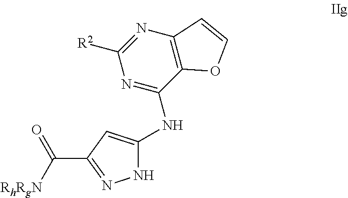

or a salt thereof.

In one embodiment X is absent.

A specific value for X is O.

Another specific value for X is NH.

Another specific value for Y is heteroaryl, wherein any heteroaryl of Y may be optionally substituted with one or more Rₐ groups.

A specific value for Y is heteroaryl.

Another specific value for Y is pyrazolyl, pyrimidinyl, thiazolyl or oxazolyl, wherein any pyrazolyl, pyrimidinyl, thiazolyl or oxazolyl of Y may be optionally substituted with one or more Rₐ groups.

Another specific value for Y is pyrazolyl, pyrimidinyl, thiazolyl or oxazolyl.

Another specific value for Y is:

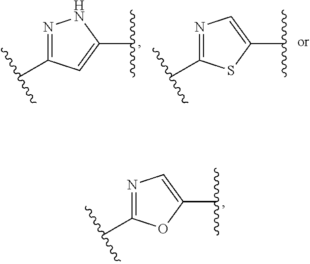

wherein the ring can be oriented in either direction in formula I.

Another specific value for Y is:

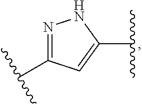

wherein the ring can be oriented in either direction in formula I.

Another specific value for Y is aryl, wherein any aryl of Y may be optionally substituted with one or more Rₐ groups.

Another specific value for Y is aryl.

Another specific value for Y is phenyl.

A specific value for R¹ is —C(O)NR_{g}Rₕ, —NRᵢC(O)NR_{g}Rₕ, —C(O)Rⱼ, or R¹ is absent.

Another specific value for R¹ is —C(O)NR_{g}Rₕ or —C(O)Rⱼ.

Another specific value for R¹ is —C(O)NR_{g}Rₕ.

A specific value for R_{g} is (C₁-C₈)alkyl, (C₃-C₈)cycloalkyl, aryl or heteroaryl.

Another specific value for R_{g} is (C₁-C₈)alkyl, (C₃-C₈)cycloalkyl, aryl or heteroaryl, wherein any aryl or heteroaryl of R_{g} may be optionally substituted with one or more Rₖ groups and wherein any alkyl or cycloalkyl of R_{g} may be optionally substituted with one or more oxo (C═O) or Rₖ groups.

Another specific value for R_{g} is (C₅-C₈)alkyl, (C₅-C₈)cycloalkyl, aryl or heteroaryl.

Another specific value for R_{g} is (C₅-C₈)alkyl, (C₅-C₈)cycloalkyl, aryl or heteroaryl, wherein any aryl or heteroaryl of R_{g} may be optionally substituted with one or more Rₖ groups and wherein any alkyl or cycloalkyl of R_{g} may be optionally substituted with one or more oxo (C═O) or Rₖ groups.

Another specific value for R_{g} is (C₁-C₆)alkyl or (C₃-C₆)cycloalkyl.

Another specific value for R_{g} is (C₁-C₆)alkyl or (C₃-C₆)cycloalkyl, wherein any alkyl or cycloalkyl of R_{g} may be optionally substituted with one or more oxo (C═O) or Rₖ groups.

Another specific value for R_{g} is (C₁-C₄)alkyl or (C₃-C₆)cycloalkyl.

Another specific value for R_{g} is (C₁-C₄)alkyl or (C₃-C₆)cycloalkyl, wherein any alkyl or cycloalkyl of R_{g} may be optionally substituted with one or more oxo (C═O) or Rₖ groups.

Another specific value for R_{g} is (C₅-C₈)alkyl or (C₅-C₈)cycloalkyl.

Another specific value for R_{g} is (C₅-C₈)alkyl or (C₅-C₈)cycloalkyl, wherein any alkyl or cycloalkyl of R_{g} may be optionally substituted with one or more oxo (C═O) or Rₖ groups.

Another specific value for R_{g} is aryl, wherein any aryl of R_{g} may be optionally substituted with one or more Rₖ groups.

Another specific value for R_{g} is heteroaryl, wherein any heteroaryl of R_{g} may be optionally substituted with one or more Rₖ groups.

Another specific value for R_{g} is aryl or heteroaryl, wherein any aryl or heteroaryl of R_{g} may be optionally substituted with one or more Rₖ groups.

Another specific value for R_{g} is heterocycle, wherein any heterocycle of R_{g} of may be optionally substituted with one or more oxo (C═O) or Rₖ groups.

A specific value for Rₕ is H or (C₁-C₆)alkyl, wherein any alkyl of Rₕ may be optionally substituted with one or more oxo (C═O) or Rₖ groups

Another specific value for R_{g} is aryl or heteroaryl.

Another specific value for R_{g} is aryl.

Another specific value for R_{g} is heteroaryl.

Another specific value for R_{g} is heterocycle.

Another specific value for Rₕ is H or (C₁-C₆)alkyl.

Another specific value for Rₕ is H.

A specific value for —X—Y—R¹ is:

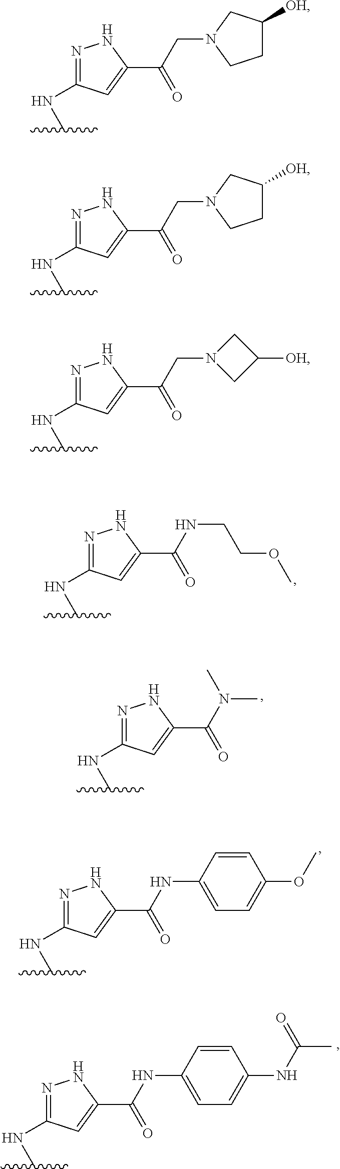

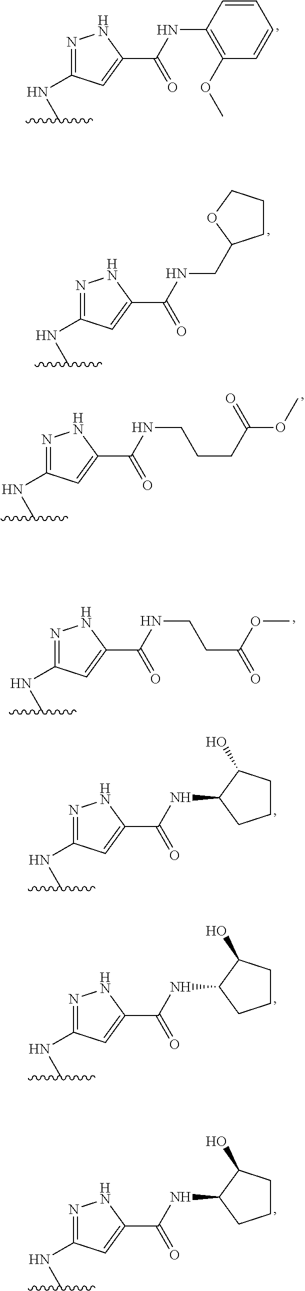

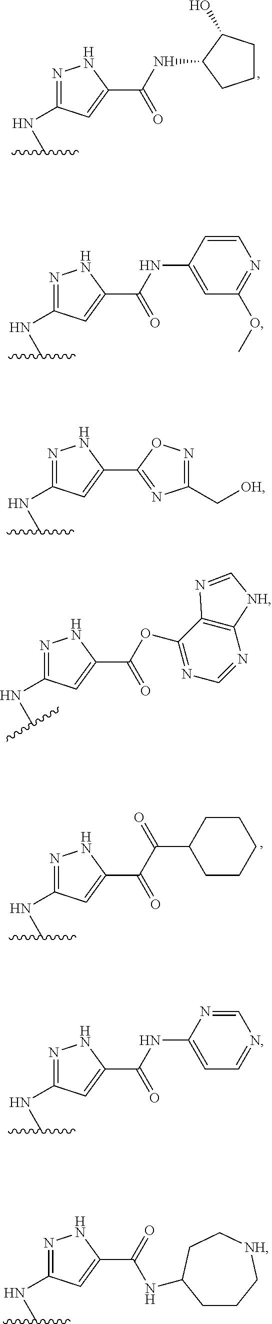

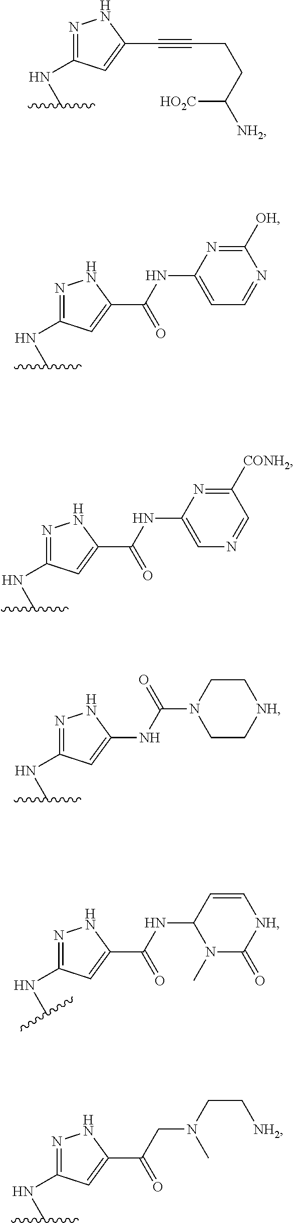

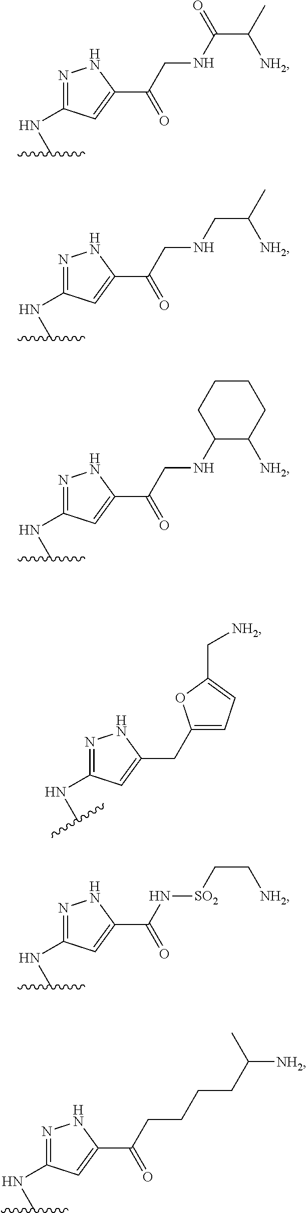

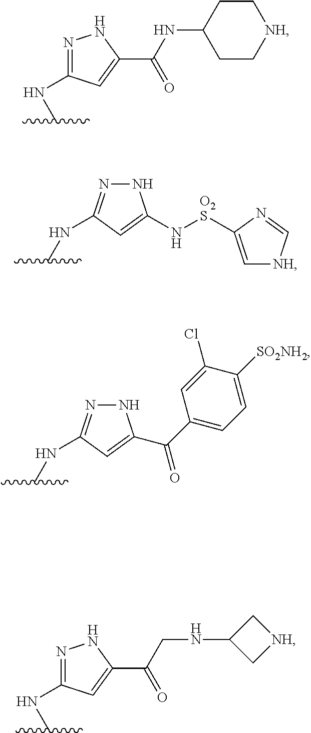

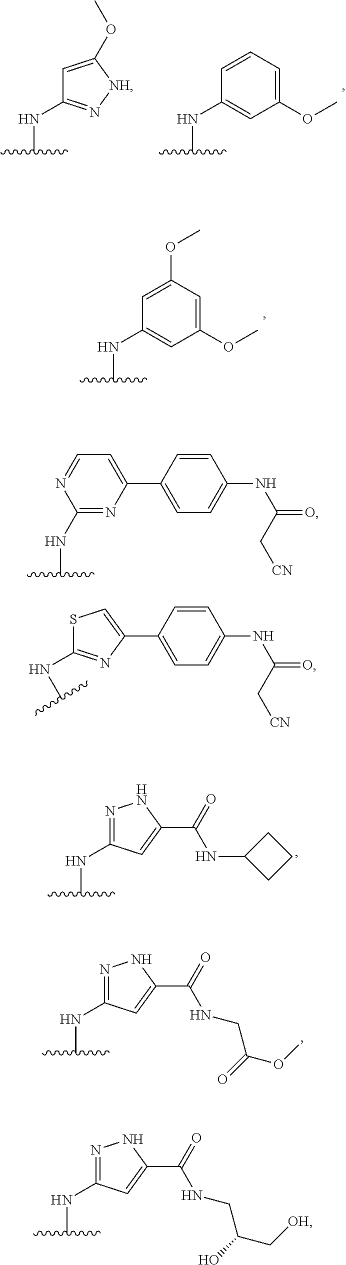

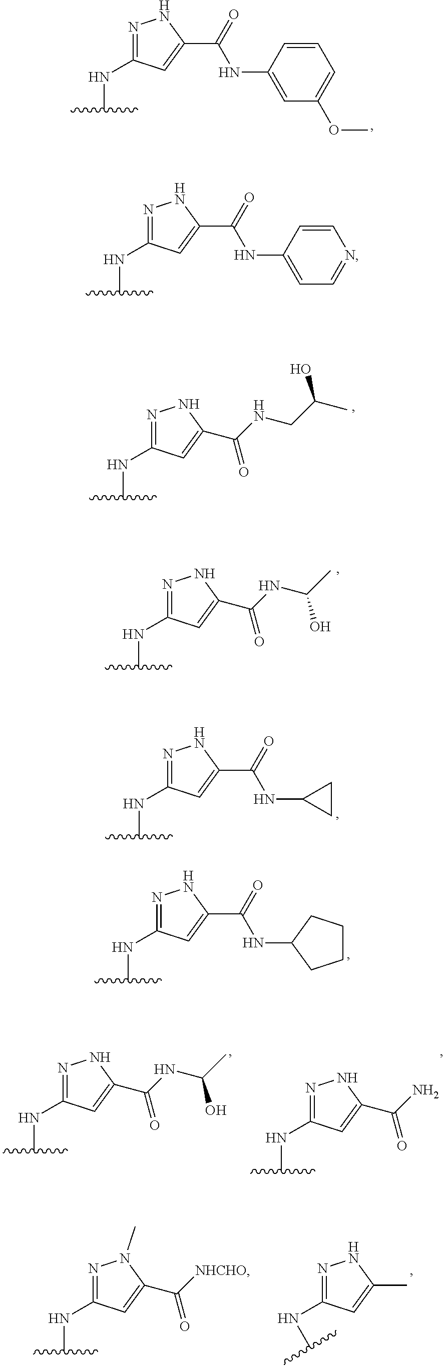

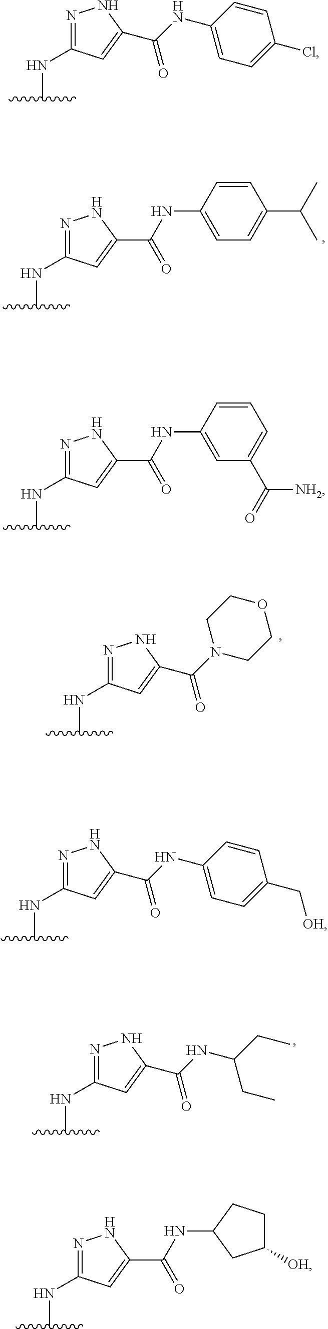

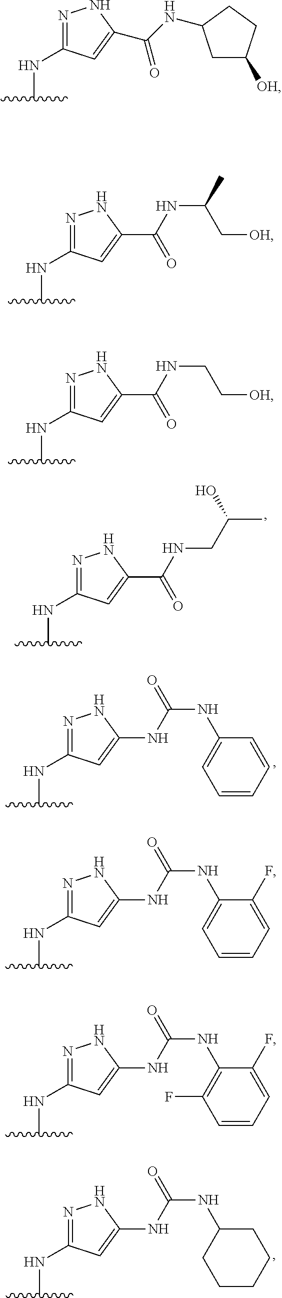

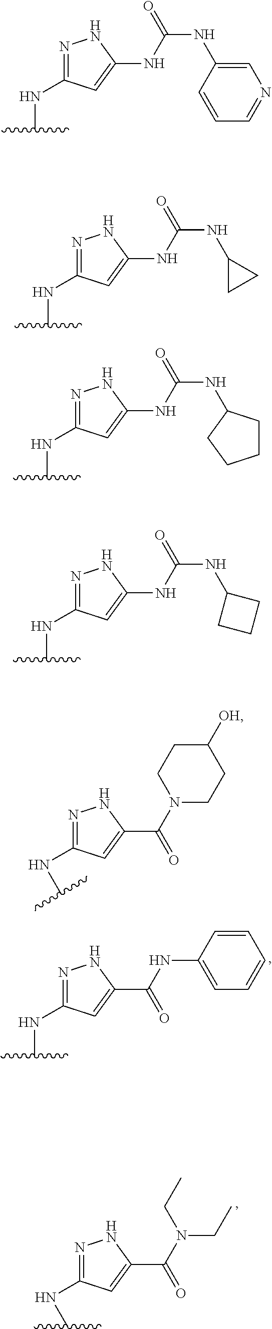

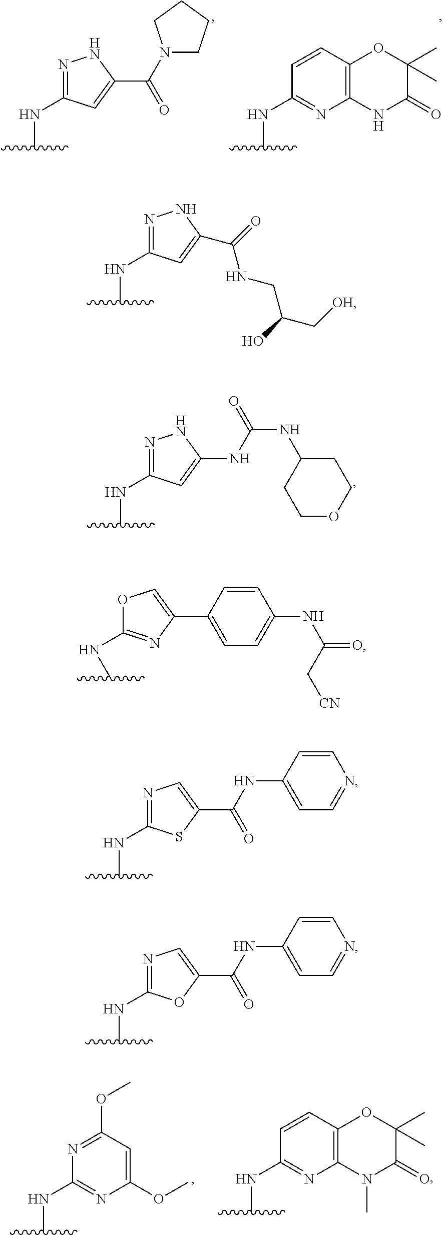

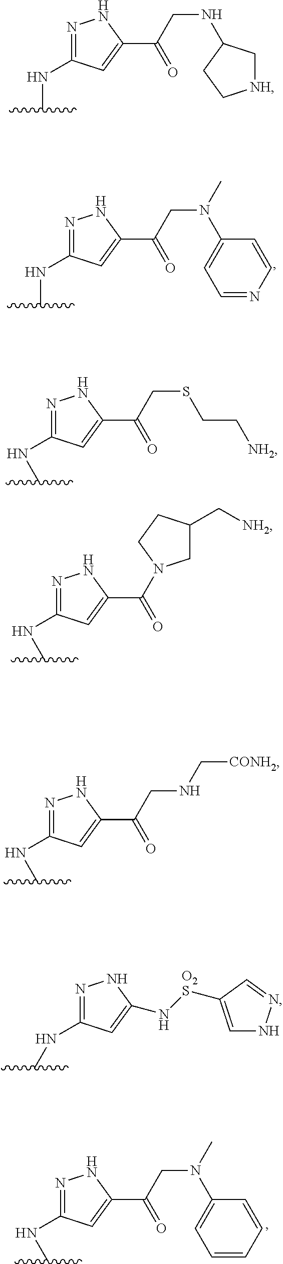

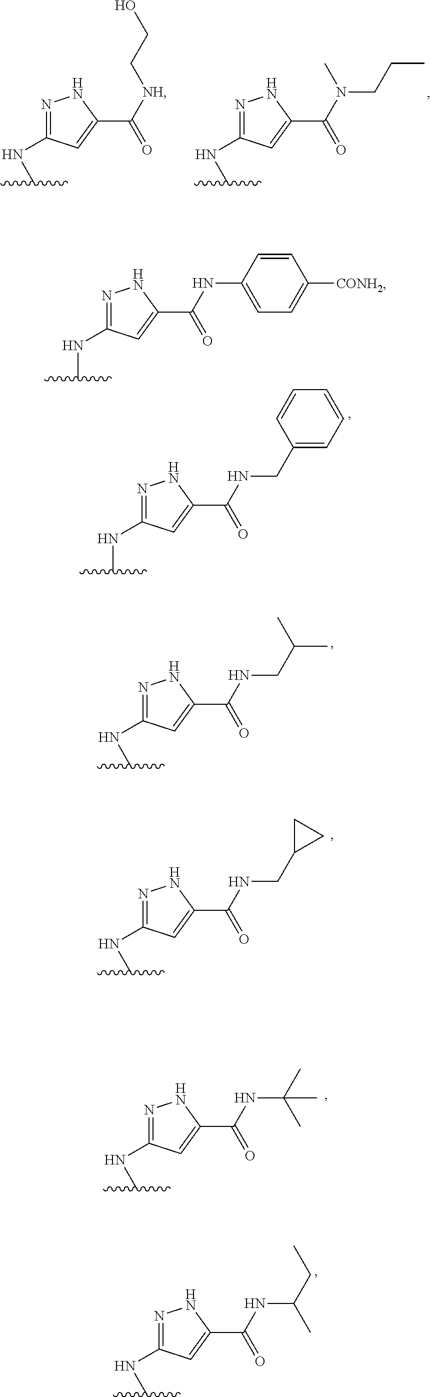

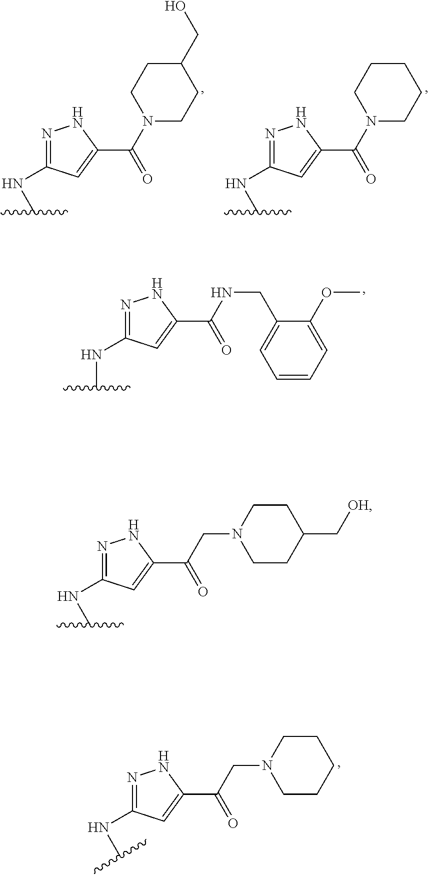

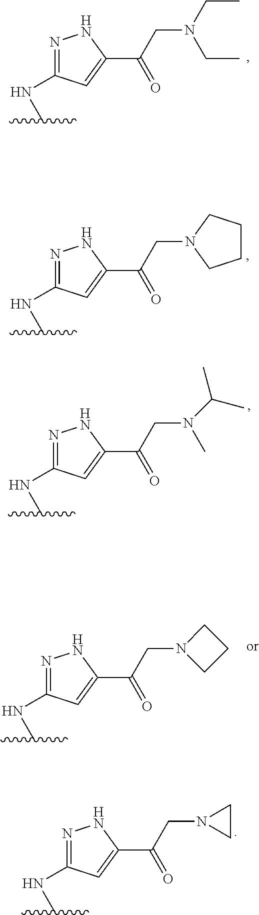

Another specific value for —X—Y—R¹ is:

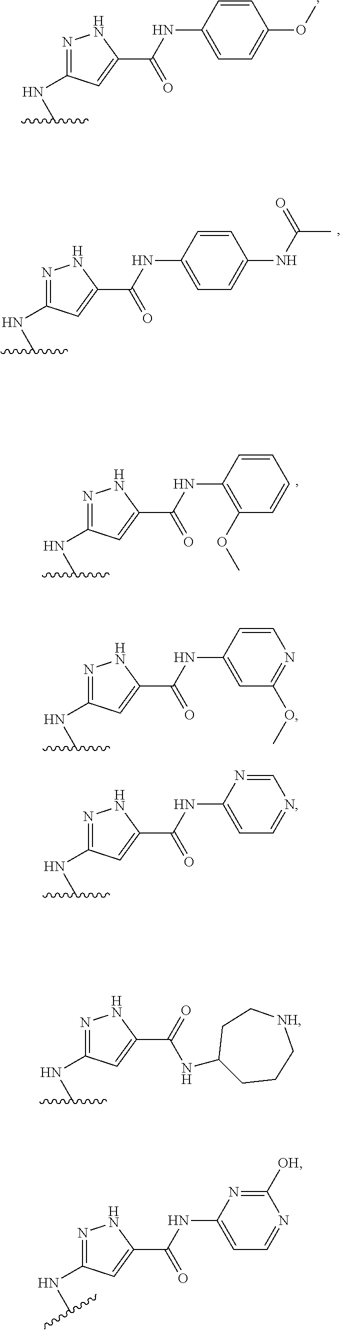

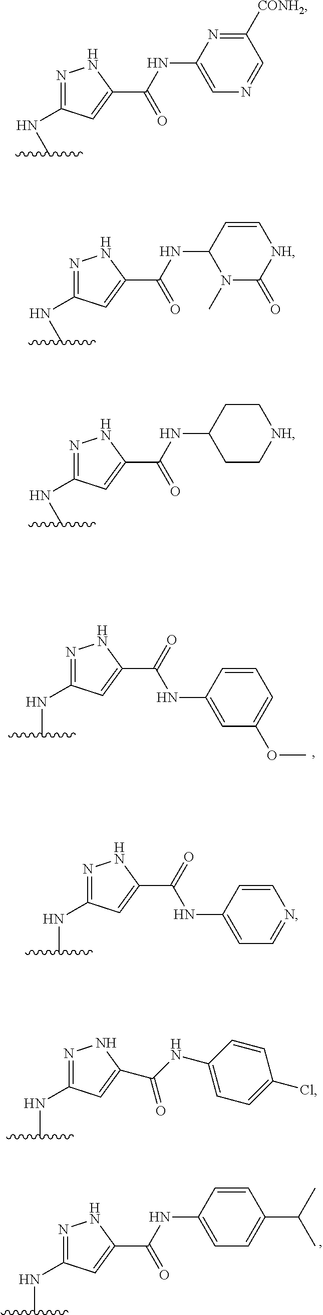

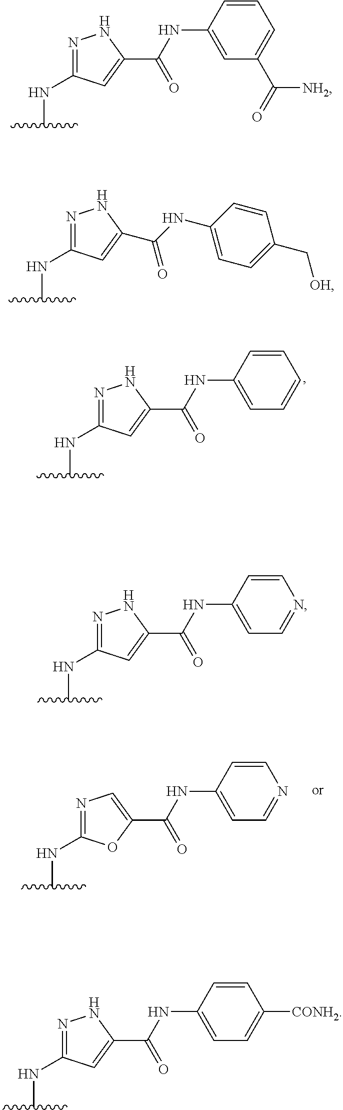

Another specific value for —X—Y—R¹ is:

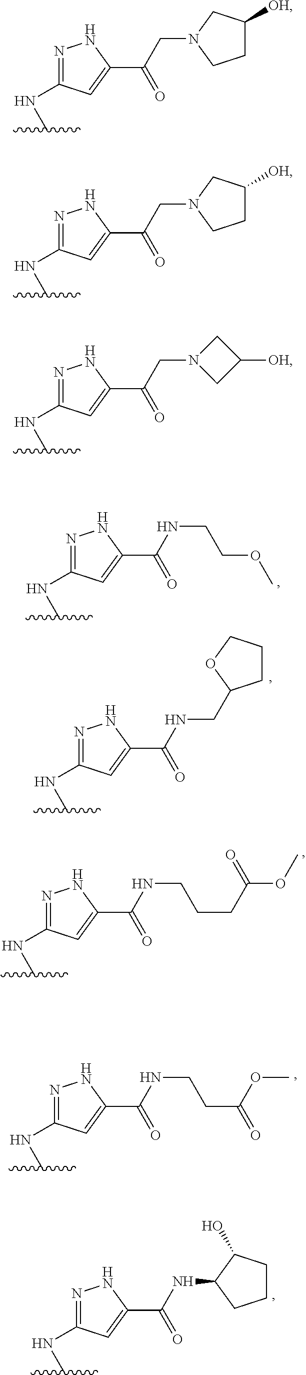

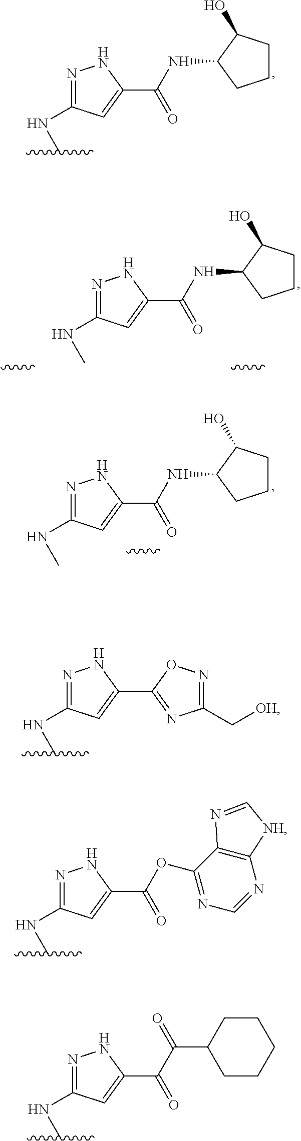

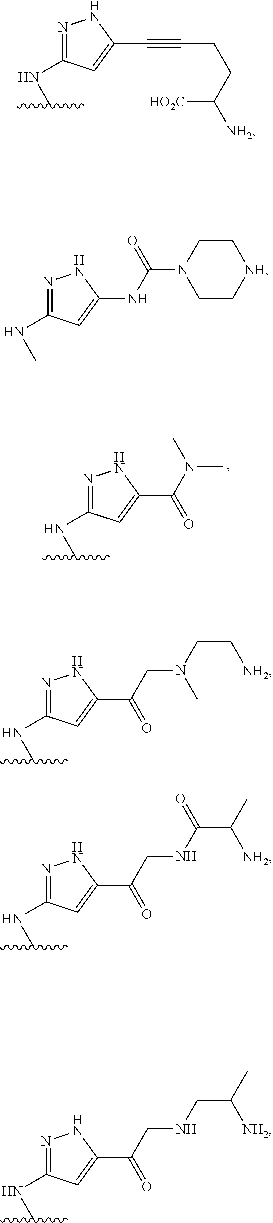

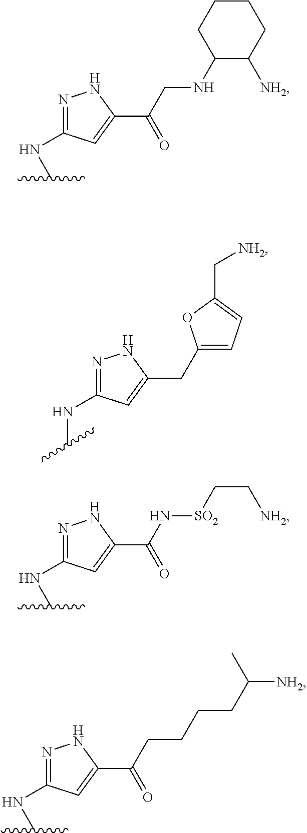

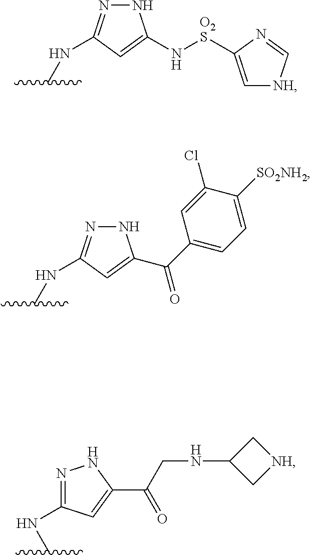

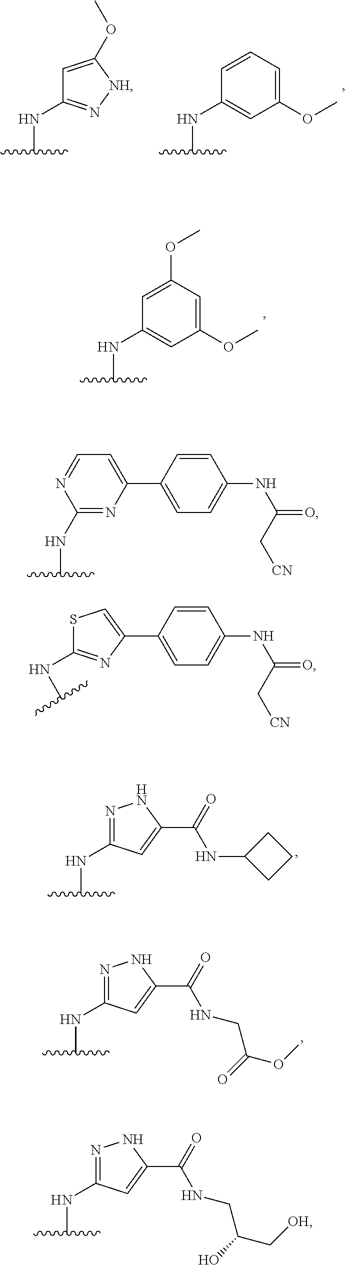

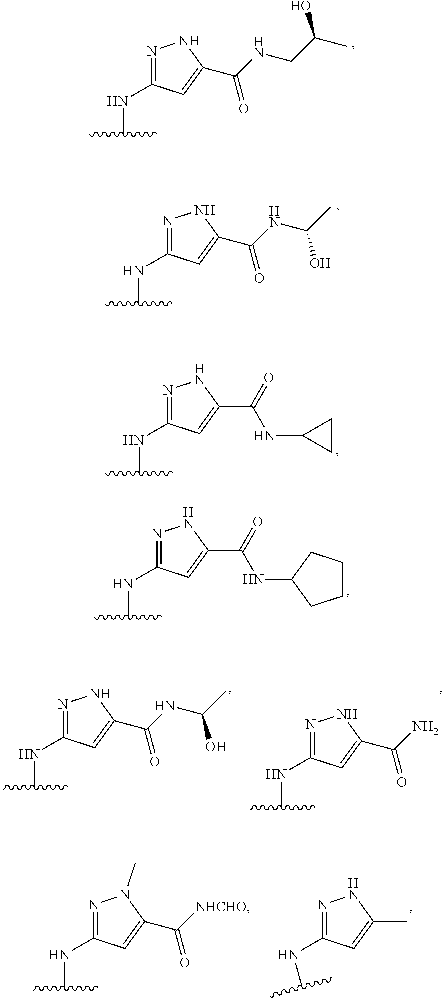

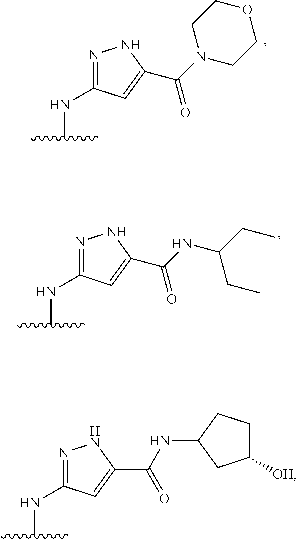

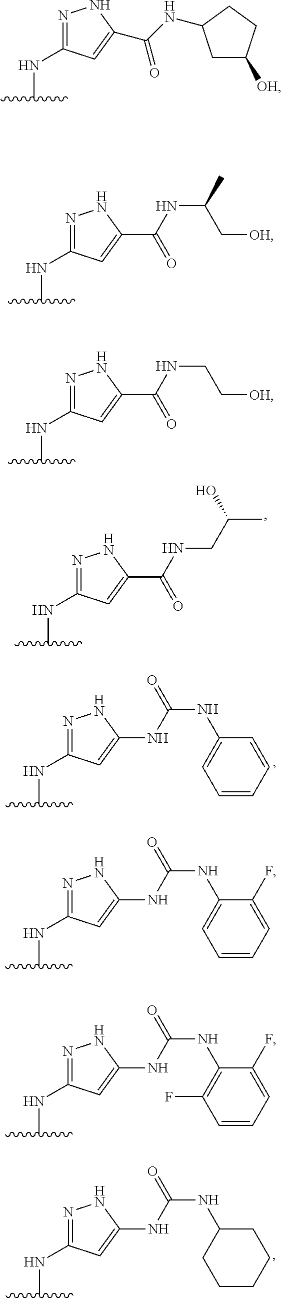

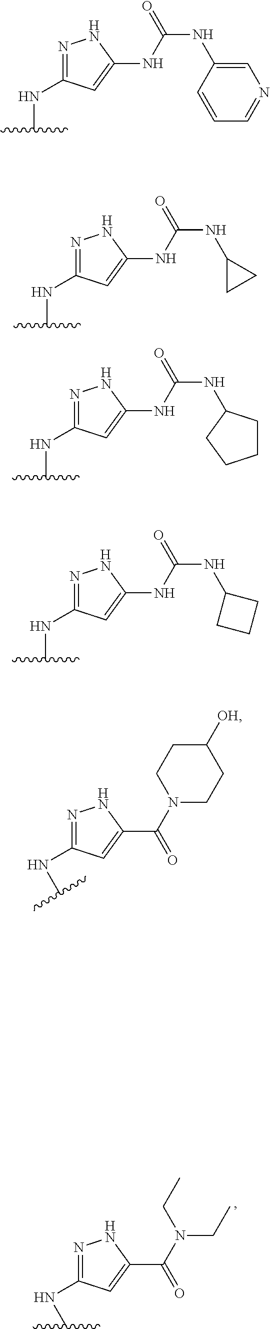

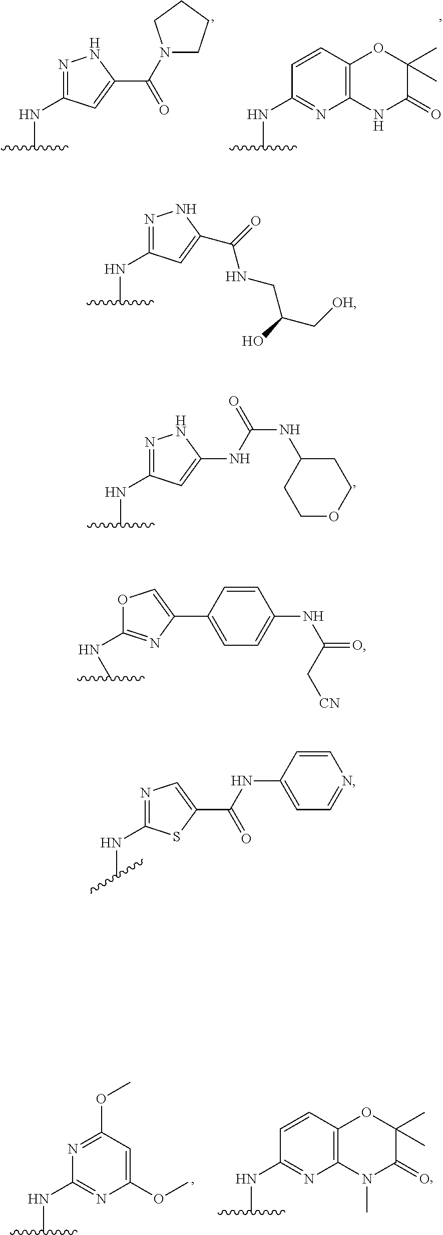

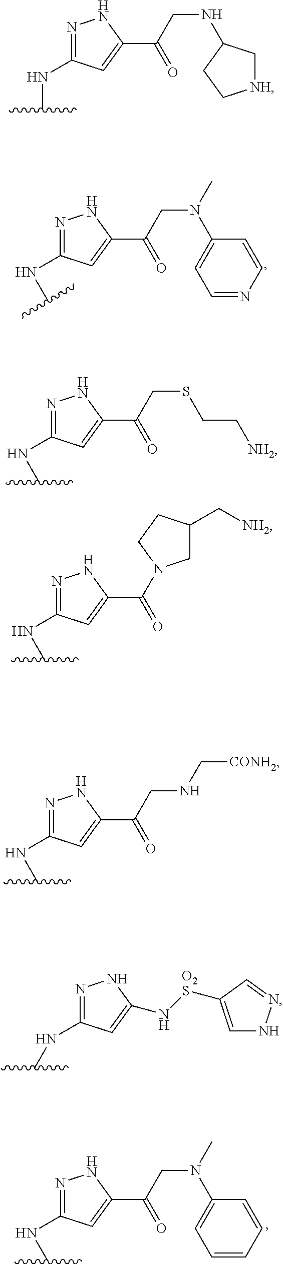

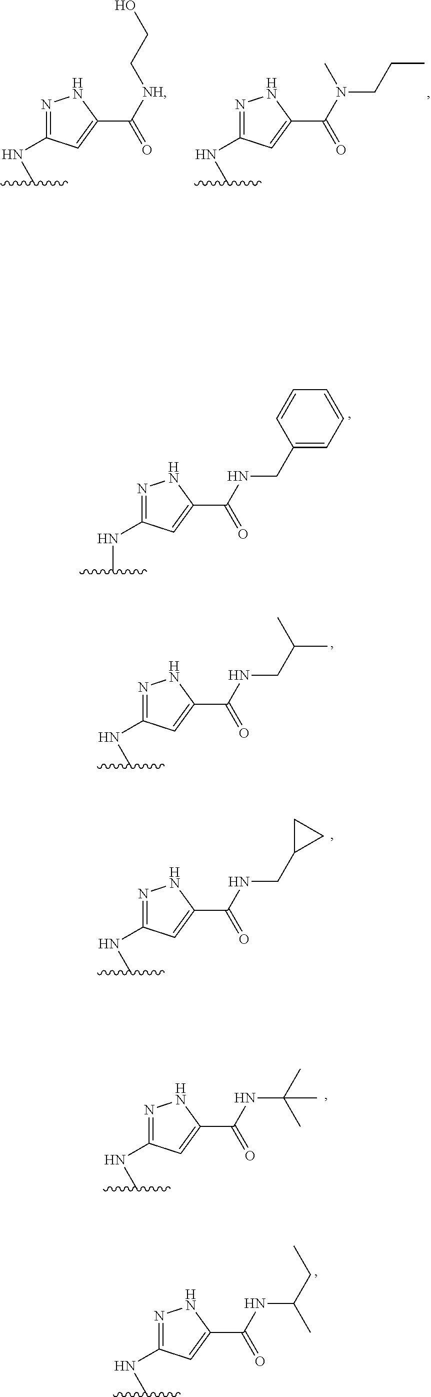

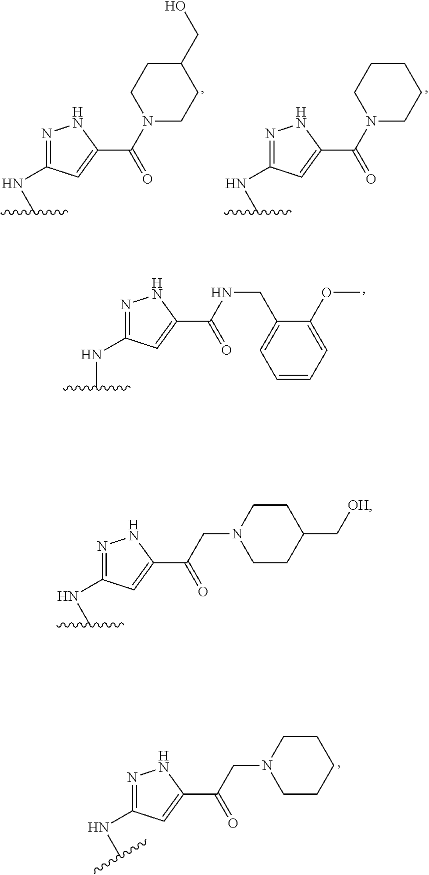

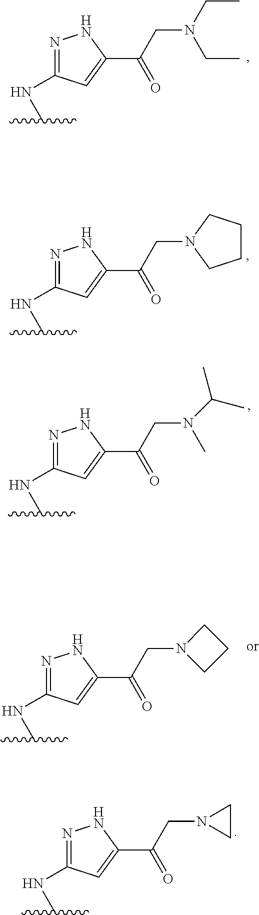

A specific value for R² is —NR⁶R⁷ or —OR⁸.

Another specific value for R² is —OR⁸.

A specific value for R⁸ is (C₁-C₆)alkyl.

A specific value for —NR⁶R⁷ is pyrrolidino, piperidino, piperazino, azetidino, morpholino or thiomorpholino, wherein any pyrrolidino, piperidino, piperazino, azetidino, morpholino or thiomorpholino of R⁶ and R⁷ may be optionally substituted with one or more R¹¹ groups.

Another specific value for —NR⁶R⁷ is pyrrolidino, piperidino, piperazino, azetidino, morpholino or thiomorpholino.

A specific value for R⁶ is (C₁-C₆)alkyl or (C₃-C₆)cycloalkyl, wherein any alkyl or cycloalkyl of R⁶ may be optionally substituted with one or more R¹¹ groups.

A specific value for R⁶ is (C₁-C₆)alkyl or (C₃-C₆)cycloalkyl.

A specific value for R⁷ is H.

Another specific group of compounds of formula I are compounds wherein —NR⁶R⁷ is pyrrolidino substituted with one or two R¹¹ groups.

Another specific group of compounds of formula I are compounds wherein R² is:

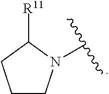

A specific value for R¹¹ is heteroaryl, aryl, —CH₂OH, —CH₂NH₂, —NHC(O)CH₃ and OH.

Another specific value for R¹¹ is heteroaryl.

Another specific value for R¹¹ is pyridine.

Another specific value for R¹¹ is —CH₂OH.

Another specific value for R² is:

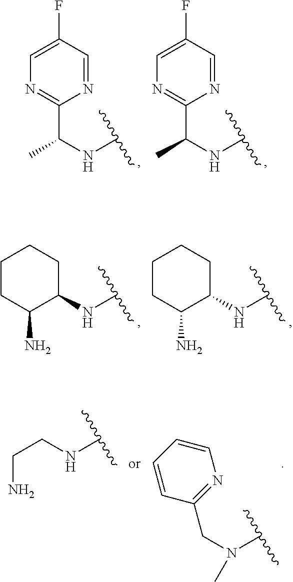

Another specific value for R² is:

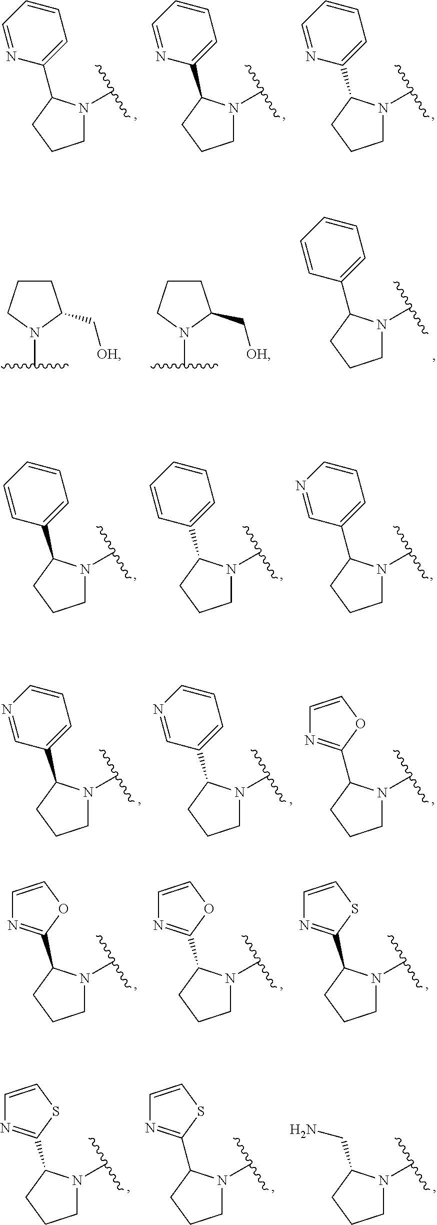

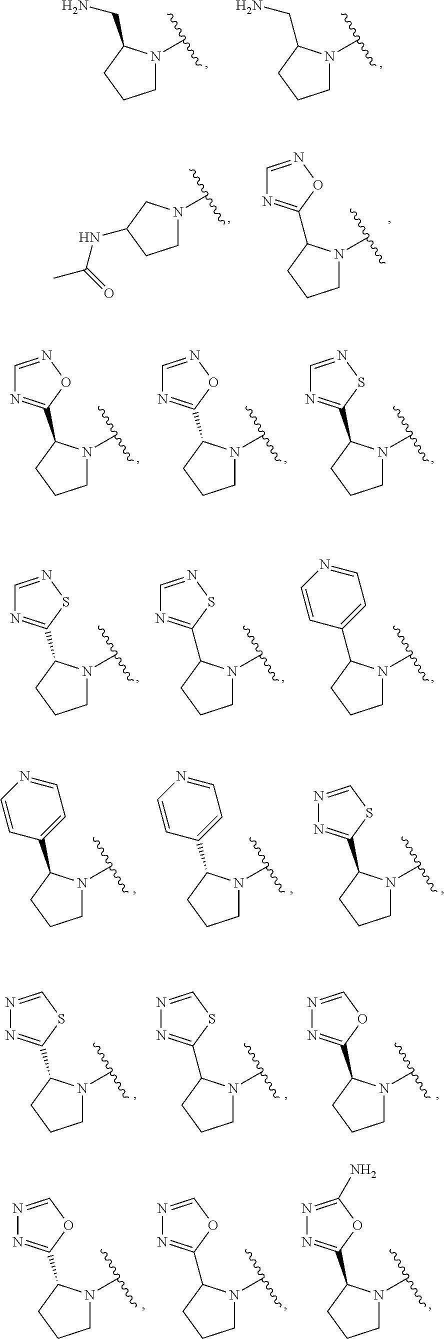

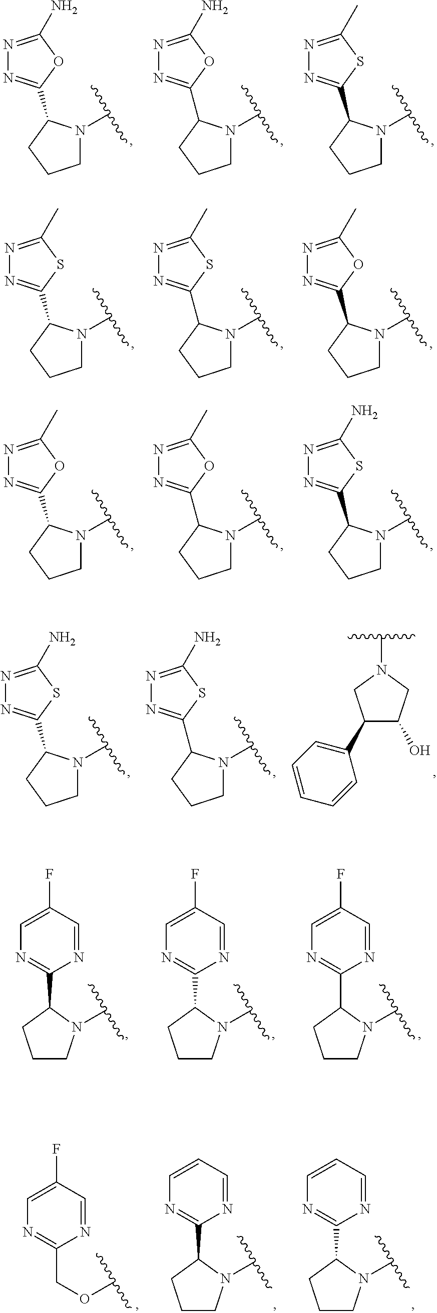

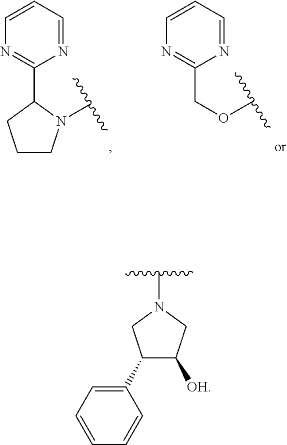

Another specific value for R² is:

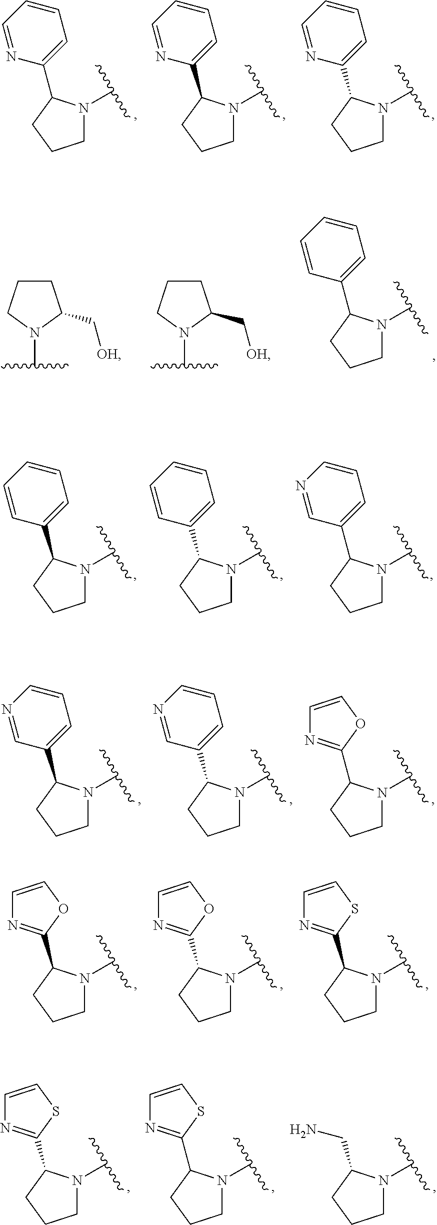

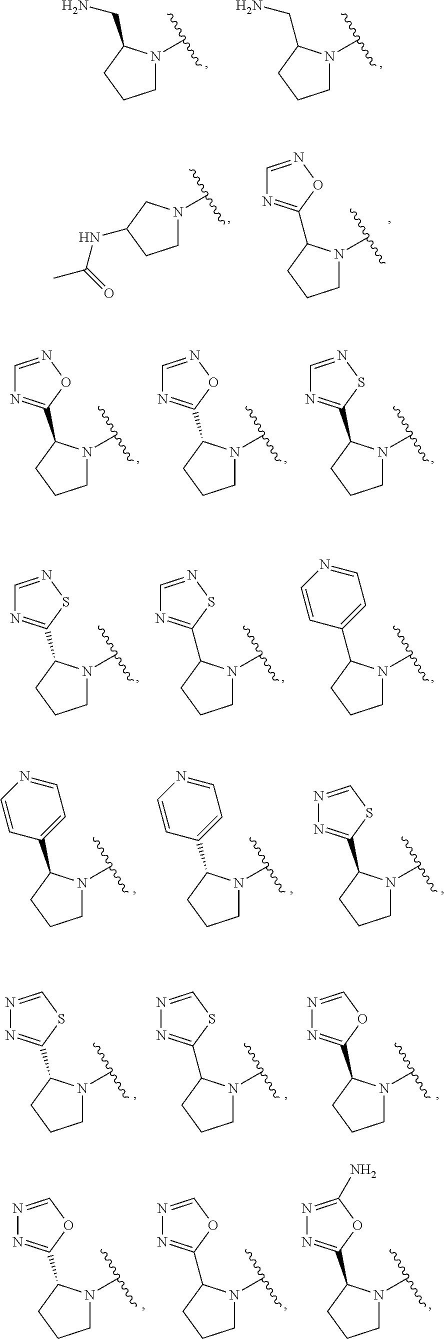

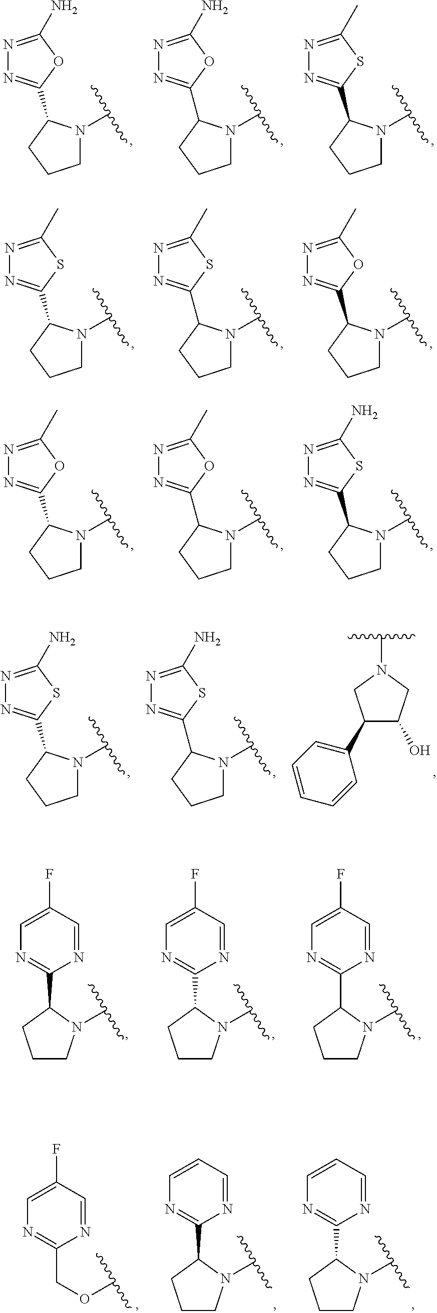

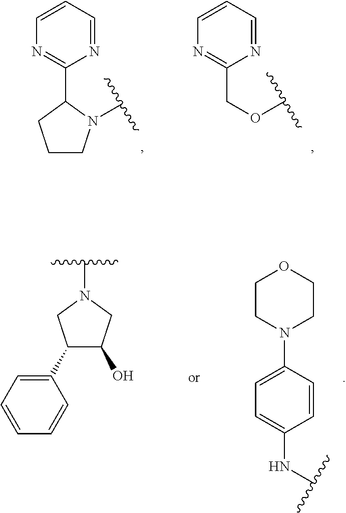

In one embodiment, the invention provides a specific group of compounds of formula I wherein:

A is furan optionally substituted with one or more (e.g. 1 or 2) R³ groups;

X is NH, O, S or absent;

Y is heteroaryl or aryl wherein heteroaryl is linked to X by a carbon atom when X is NH, O or S and wherein any heteroaryl or aryl of Y may be optionally substituted with one or more (e.g. 1, 2, 3, 4 or 5) Rₐ groups;

R¹ is —C(O)NR_{g}Rₕ, —C(S)NR_{g}Rₕ, or —C(═NRᵢ)NR_{g}Rₕ;

R² is heteroaryl, —NR⁶R⁷, —OR⁸, SR⁸ or CHR⁹R¹⁰ wherein any heteroaryl of R² may be optionally substituted with one or more (e.g. 1, 2 or 3) R¹¹ groups;

each R³ is independently halo, (C₁-C₆)alkyl, (C₂-C₆)alkenyl, (C₂-C₆)alkynyl, (C₃-C₆)cycloalkyl, —ORₐ₂, —OC(O)R_{b2}, —OC(O)NR_{c2}R_{d2}, —SRₐ₂, —S(O)₂OH, —S(O)R_{b2}, —S(O)₂R_{b2}, —S(O)₂NR_{c2}R_{d2}, —NR_{c2}R_{d2}, —NRₑ₂C(O)R_{b2}, —NRₑ₂C(O)NR_{c2}R_{d2}, NRₑ₂S(O)₂R_{b2}, —NRₑ₂S(O)₂NR_{c2}R_{d2}, NO₂, —C(O)Rₐ₂, —C(O)ORₐ₂ or —C(O)NR_{c2}R_{d2};

R⁶ is selected from (C₁-C₆)alkyl, (C₂-C₆)alkenyl, (C₂-C₆)alkynyl, (C₃-C₆)cycloalkyl, heteroaryl, heterocycle and aryl; and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino; wherein any alkyl, alkenyl, alkynyl, cycloalkyl, heteroaryl, heterocycle, aryl pyrrolidino, piperidino, piperazino, azetidino, morpholino or thiomorpholino of R⁶ and R⁷ may be optionally substituted with one or more (e.g. 1, 2 or 3) R¹¹ groups;

each R⁸ is independently selected from (C₁-C₆)alkyl, (C₂-C₆)alkenyl, (C₂-C₆)alkynyl, (C₃-C₆)cycloalkyl, heteroaryl and aryl wherein any alkyl, alkenyl, alkynyl, cycloalkyl, heteroaryl or aryl of R⁸ may be optionally substituted with one or more (e.g. 1, 2 or 3) R¹¹ groups;

R⁹ is selected from (C₁-C₆)alkyl, (C₂-C₆)alkenyl, (C₂-C₆)alkynyl, (C₃-C₆)cycloalkyl, heteroaryl, heterocycle and aryl; and R¹⁰ is selected from H and (C₁-C₆)alkyl; or R⁹ and R¹⁰ together with the carbon to which they are attached form a (C₃-C₇)cycloalkyl, pyrrolidino, piperidino, piperazino, azetidino, morpholino, or thiomorpholino wherein any alkyl, alkenyl, alkynyl, cycloalkyl, heteroaryl, heterocycle, aryl pyrrolidino, piperidino, piperazino, azetidino, morpholino, or thiomorpholino of R⁹ and R¹⁰ may be optionally substituted with one or more (e.g. 1, 2 or 3) R¹¹ groups;

each R¹¹ is independently selected from (C₁-C₆)alkyl, (C₂-C₆)alkenyl, (C₂-C₆)alkynyl, (C₃-C₆)cycloalkyl, —ORₘ, —NRₜCORₙ, NRₒRₚ, heteroaryl and aryl wherein alkyl, alkenyl, alkynyl, cycloalkyl, heteroaryl or aryl may be optionally substituted with one or more (e.g. 1, 2, 3, 4 or 5) groups selected from halo, R_{q}, OH, CN, —OR_{q}, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, CHO, —C(O)R_{q}, CO₂H, —C(O)OR_{q} and —C(O)NRᵣRₛ;

each Rₐ is independently selected from (C₁-C₆)alkyl, (C₂-C₆)alkenyl, (C₂-C₆)alkynyl, (C₃-C₆)cycloalkyl, halo, CN, —OR_{f}, —OC(O)R_{b}, —OC(O)NR_{c}R_{d}, —SR_{f}, —S(O)R_{b}, —S(O)₂OH, —S(O)₂R_{b}, —S(O)₂NR_{c}R_{d}, —NR_{c}R_{b}, —NRₑCOR_{b}, —NRₑCO₂R_{b}, —NRₑCONR_{c}R_{d}, —NRₑS(O)₂R_{b}, —NRₑS(O)₂NR_{c}R_{d}, NO₂, —C(O)R_{f}, —C(O)OR_{f} and —C(O)NR_{c}R_{d};

each R_{b} is independently (C₁-C₆)alkyl, (C₂-C₆)alkenyl, (C₂-C₆)alkynyl, (C₃-C₆)cycloalkyl, heterocycle, heteroaryl or aryl;

R_{c} and R_{d} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c} and R_{d} together with the nitrogen to which they are attached form a pyrrolidino, piperidino, piperazino, azetidino, morpholino, or thiomorpholino;

each Rₑ is independently H, (C₁-C₆)alkyl, (C₂-C₆)alkenyl, (C₂-C₆) alkynyl or (C₃-C₆)cycloalkyl;

each R_{f} is independently H, (C₁-C₆)alkyl, (C₂-C₆)alkenyl, (C₂-C₆)alkynyl, (C₃-C₆)cycloalkyl, heterocycle, heteroaryl or aryl;

each R_{g} is independently selected from aryl, heterocycle and heteroaryl wherein any aryl or heteroaryl of R_{g} may be optionally substituted with one or more (e.g. 1, 2, 3, 4 or 5) Rₖ groups and wherein any heterocycle of R_{g} may be optionally substituted with one or more (e.g. 1, 2, 3, 4 or 5) oxo (C═O) or Rₖ groups;

each Rₕ is independently selected from H, (C₁-C₈)alkyl, (C₂-C₈)alkenyl, (C₂-C₈)alkynyl, (C₃-C₈)cycloalkyl, heterocycle, heteroaryl and aryl wherein any aryl or heteroaryl of Rₕ may be optionally substituted with one or more (e.g. 1, 2, 3, 4 or 5) Rₖ groups and wherein any alkyl, alkenyl, alkynyl, cycloalkyl or heterocycle of Rₕ may be optionally substituted with one or more (e.g. 1, 2, 3, 4 or 5) oxo (C═O) or Rₖ groups;

Rᵢ is independently H, (C₁-C₆)alkyl, (C₂-C₆)alkenyl, (C₂-C₆)alkynyl or (C₃-C₆)cycloalkyl;

each Rₖ is independently selected from halo, R_{y}, CN, OH, —OR_{y}, —OC(O)R_{y}, —OC(O)NRᵥR_{w}, SH, —SR_{y}, —S(O)R_{y}, —S(O)₂OH, —S(O)₂R_{y}, —S(O)₂NRᵥR_{w}, —NRᵥR_{w}, —NRₓCOR_{y}, —NRₓCO₂R_{y}, —NRₓCONRᵥR_{w}, —NRₓS(O)₂R_{y}, —NRₓS(O)₂NRᵥR_{w}, NO₂, —C(O)Rᵤ, —C(O)ORᵤ and —C(O)NRᵥR_{w};

each Rₘ is independently H, (C₁-C₆)alkyl, (C₂-C₆)alkenyl, (C₂-C₆)alkynyl, (C₃-C₆)cycloalkyl, heterocycle, heteroaryl or aryl;

each Rₙ is independently (C₁-C₆)alkyl, (C₂-C₆)alkenyl, (C₂-C₆)alkynyl, (C₃-C₆)cycloalkyl, heterocycle, heteroaryl or aryl;

Rₒ and Rₚ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rₒ and Rₚ together with the nitrogen to which they are attached form a pyrrolidino, piperidino, piperazino, azetidino, morpholino, or thiomorpholino;

each R_{q} is independently (C₁-C₆)alkyl, (C₂-C₆)alkenyl, (C₂-C₆)alkynyl, (C₃-C₆)cycloalkyl, heterocycle, heteroaryl or aryl;

Rᵣ and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino;

each Rₜ is independently H, (C₁-C₆)alkyl, (C₂-C₆)alkenyl, (C₂-C₆) alkynyl or (C₃-C₆)cycloalkyl;

each Rᵤ is independently H, (C₁-C₆)alkyl, (C₂-C₆)alkenyl, (C₂-C₆)alkynyl, (C₃-C₆)cycloalkyl, heterocycle, heteroaryl or aryl;

R^{v} and R_{w} are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵥ and R_{w} together with the nitrogen to which they are attached form a pyrrolidino, piperidino, piperazino, azetidino, morpholino, or thiomorpholino wherein any alkyl, alkenyl, alkynyl, cycloalkyl, heteroaryl, heterocycle, aryl pyrrolidino, piperidino, piperazino, azetidino, morpholino, or thiomorpholino of Rᵥ and R_{w} may be optionally substituted with one or more (e.g. 1 or 2) groups independently selected from OH, CH₂OH, NH₂ and CONH₂;

each Rₓ is independently H, (C₁-C₆)alkyl, (C₂-C₆)alkenyl, (C₂-C₆) alkynyl or (C₃-C₆)cycloalkyl;

each R_{y} is independently (C₁-C₆)alkyl, (C₂-C₆)alkenyl, (C₂-C₆)alkynyl, (C₃-C₆)cycloalkyl, heterocycle, heteroaryl or aryl wherein any alkyl, alkenyl, alkynyl, cycloalkyl, heterocycle, heteroaryl or aryl of R_{y} may be optionally substituted with one or more (e.g. 1 or 2) groups selected from ORᵤ and NRᵥR_{w};

each Rₐ₂ is independently H, (C₁-C₆)alkyl, (C₂-C₆)alkenyl, (C₂-C₆)alkynyl, (C₃-C₆)cycloalkyl, heterocycle, heteroaryl or aryl;

each R_{b2} is independently (C₁-C₆)alkyl, (C₂-C₆)alkenyl, (C₂-C₆)alkynyl, (C₃-C₆)cycloalkyl, heterocycle, heteroaryl or aryl;

R_{c2} and R_{d2} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c2} and R_{d2} together with the nitrogen to which they are attached form a pyrrolidino, piperidino, piperazino, azetidino, morpholino, or thiomorpholino; and

each Rₑ₂ is independently H, (C₁-C₆)alkyl, (C₂-C₆)alkenyl, (C₂-C₆) alkynyl or (C₃-C₆)cycloalkyl;

or a salt thereof.

In another embodiment, the invention provides a specific group of compounds of formula I wherein:

A is furan optionally substituted with one or more R³ groups;

X is NH, O, S or absent;

Y is heteroaryl or aryl wherein heteroaryl is linked to X by a carbon atom when X is NH, O or S and wherein any heteroaryl or aryl of Y may be optionally substituted with one or more Rₐ groups;

R¹ is —C(O)NR_{g1}Rₕ₁, —NRᵢC(O)NR_{g}Rₕ, —CHO, —C(O)Rⱼ, —CO₂H, —C(O)ORⱼ, —NRᵢS(O)₂NR_{g}Rₕ, —NRᵢC(O)Rⱼ, —NRᵢS(O)₂Rⱼ, —C(O)C(O)Rⱼ, —C(O)NRᵢS(O)₂Rⱼ, —C(O)NRᵢCHO, —C(O)NRᵢC(O)Rⱼ, —C≡CH, —C≡CRⱼ, —C(S)NR_{g1}Rₕ₁, —C(═NRᵢ)NR_{g1}Rₕ₁, (C₁-C₆)alkyl, (C₃-C₆)cycloalkyl, heterocycle, heteroaryl, aryl or is absent and wherein any alkyl, cycloalkyl, heterocycle, heteroaryl or aryl of R¹ may be optionally substituted with one or more R^{z} groups;

R² is heteroaryl, —NR⁶R⁷, —OR⁸, SR⁸ or CHR⁹R¹⁰ wherein any heteroaryl of R² may be optionally substituted with one or more R¹¹ groups;

each R³ is independently halo, (C₁-C₆)alkyl, (C₂-C₆)alkenyl, (C₂-C₆)alkynyl, (C₃-C₆)cycloalkyl, —ORₐ₂, —OC(O)R_{b2}, —OC(O)NR_{c2}R_{d2}, —SRₐ₂, —S(O)₂OH, —S(O)R_{b2}, —S(O)₂R_{b2}, —S(O)₂NR_{c2}R_{d2}, —NR_{c2}R_{d2}, —NRₑ₂C(O)R_{b2}, —NRₑ₂C(O)NR_{c2}R_{d2}, NRₑ₂S(O)₂R_{b2}, —NRₑ₂S(O)₂NR_{c2}R_{d2}, NO₂, —C(O)Rₐ₂, —C(O)ORₐ₂ or —C(O)NR_{c2}R_{d2};

R⁶ is selected from (C₁-C₆)alkyl, (C₂-C₆)alkenyl, (C₂-C₆)alkynyl, (C₃-C₆)cycloalkyl, heteroaryl, heterocycle and aryl; and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino; wherein any alkyl, alkenyl, alkynyl, cycloalkyl, heteroaryl, heterocycle, aryl pyrrolidino, piperidino, piperazino, azetidino, morpholino or thiomorpholino of R⁶ and R⁷ may be optionally substituted with one or more R₁₁ groups;

each R⁸ is independently selected from (C₁-C₆)alkyl, (C₂-C₆)alkenyl, (C₂-C₆)alkynyl, (C₃-C₆)cycloalkyl, heteroaryl and aryl wherein any alkyl, alkenyl, alkynyl, cycloalkyl, heteroaryl or aryl of R⁸ may be optionally substituted with one or more R¹¹ groups;

R⁹ is selected from (C₁-C₆)alkyl, (C₂-C₆)alkenyl, (C₂-C₆)alkynyl, (C₃-C₆)cycloalkyl, heteroaryl, heterocycle and aryl; and R¹⁰ is selected from H and (C₁-C₆)alkyl; or R⁹ and R¹⁰ together with the carbon to which they are attached form a (C₃-C₇)cycloalkyl, pyrrolidino, piperidino, piperazino, azetidino, morpholino, or thiomorpholino wherein any alkyl, alkenyl, alkynyl, cycloalkyl, heteroaryl, heterocycle, aryl pyrrolidino, piperidino, piperazino, azetidino, morpholino, or thiomorpholino of R⁹ and R¹⁰ may be optionally substituted with one or more R¹¹ groups;

each R¹¹ is independently selected from (C₁-C₆)alkyl, (C₂-C₆)alkenyl, (C₂-C₆)alkynyl, (C₃-C₆)cycloalkyl, —NRₜCORₙ, NRₒRₚ, heteroaryl and aryl wherein alkyl, alkenyl, alkynyl, cycloalkyl, heteroaryl or aryl may be optionally substituted with one or more groups selected from halo, R_{q}, OH, CN, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, —CHO, —C(O)R_{q}, —C(O)OH, —C(O)OR_{q} and —C(O)NRᵣRₛ;

each Rₐ is independently selected from (C₁-C₆)alkyl, (C₂-C₆)alkenyl, (C₂-C₆)alkynyl, (C₃-C₆)cycloalkyl, halo, CN, —OR_{f}, —OC(O)R_{b}, —OC(O)NR_{c}R_{d}, —SR_{f}, —S(O)R_{b}, —S(O)₂OH, —S(O)₂R_{b}, —S(O)₂NR_{c}R_{d}, —NR_{c}R_{d}, —NRₑCOR_{b}, —NRₑCO₂R_{b}, —NRₑCONR_{c}R_{d}, —NRₑS(O)₂R_{b}, —NRₑS(O)₂NR_{c}R_{d}, NO₂, —C(O)R_{f}, —C(O)OR_{f} and —C(O)NR_{c}R_{d};

each R_{b} is independently (C₁-C₆)alkyl, (C₂-C₆)alkenyl, (C₂-C₆)alkynyl, (C₃-C₆)cycloalkyl, heterocycle, heteroaryl or aryl;

R_{c} and R_{d} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c} and R_{d} together with the nitrogen to which they are attached form a pyrrolidino, piperidino, piperazino, azetidino, morpholino, or thiomorpholino;

each Rₑ is independently H, (C₁-C₆)alkyl, (C₂-C₆)alkenyl, (C₂-C₆) alkynyl or (C₃-C₆)cycloalkyl;

each R_{f} is independently H, (C₁-C₆)alkyl, (C₂-C₆)alkenyl, (C₂-C₆)alkynyl, (C₃-C₆)cycloalkyl, heterocycle, heteroaryl or aryl;

R_{g1} is selected from H, (C₁-C₈)alkyl, (C₂-C₈)alkenyl, (C₂-C₈)alkynyl or (C₃-C₈)cycloalkyl wherein any alkyl, alkenyl, alkynyl or cycloalkyl of R_{g1} may be optionally substituted with one or more oxo (C═O) or Rₖ groups; and Rₕ₁ is selected from H, (C₁-C₈)alkyl, (C₂-C₈)alkenyl, (C₂-C₈)alkynyl or (C₃-C₈)cycloalkyl wherein any alkyl, alkenyl, alkynyl or cycloalkyl of Rₕ₁ may be optionally substituted with one or more oxo (C═O) or Rₖ groups; or R_{g1} and Rₕ₁ together with the nitrogen to which they are attached form a pyrrolidino, piperidino, piperazino, azetidino, morpholino, or thiomorpholino wherein any pyrrolidino, piperidino, piperazino, azetidino, morpholino or thiomorpholino of R_{g1} and Rₕ₁ may be optionally substituted with one or more Rₖ or oxo groups;

R_{g} and Rₕ are each independently selected from H, (C₁-C₈)alkyl, (C₂-C₈)alkenyl, (C₂-C₈)alkynyl, (C₃-C₈)cycloalkyl, heterocycle, heteroaryl and aryl wherein any aryl or heteroaryl of R_{g} or Rₕ may be optionally substituted with one or more (e.g. 1, 2, 3, 4 or 5) Rₖ groups and wherein any alkyl, alkenyl, alkynyl, cycloalkyl or heterocycle of R_{g} or Rₕ may be optionally substituted with one or more (e.g. 1, 2, 3, 4 or 5) oxo (C═O) or Rₖ groups; or R_{g} and Rₕ together with the nitrogen to which they are attached form a pyrrolidino, piperidino, piperazino, azetidino, morpholino, or thiomorpholino wherein any pyrrolidino, piperidino, piperazino, azetidino, morpholino or thiomorpholino of R_{g} and Rₕ may be optionally substituted with one or more (e.g. 1, 2, 3, 4 or 5) Rₖ or oxo groups;

each Rᵢ is independently H, (C₁-C₆)alkyl, (C₂-C₆)alkenyl, (C₂-C₆)alkynyl or (C₃-C₆)cycloalkyl;

each Rⱼ is independently selected from (C₁-C₆)alkyl, (C₂-C₆)alkenyl, (C₂-C₆)alkynyl, (C₃-C₆)cycloalkyl, heterocycle, heteroaryl and aryl wherein any aryl or heteroaryl of Rⱼ may be optionally substituted with one or more Rₖ groups and wherein any alkyl, alkenyl, alkynyl, cycloalkyl or heterocycle of R₃ may be optionally substituted with one or more oxo (C═O) or Rₖ groups;

each Rₖ is independently selected from halo, R_{y}, CN, OH, —OR_{y}, —OC(O)R_{y}, —OC(O)NRᵥR_{w}, SH, —SR_{y}, —S(O)R_{y}, —S(O)₂OH, —S(O)₂R_{y}, —S(O)₂NRᵥR_{w}, —NRₓCOR_{y}, —NRₓCO₂R_{y}, —NRₓCONRᵥR_{w}, —NRₓS(O)₂R_{y}, —NRₓS(O)₂NRᵥR_{w}, NO₂, —C(O)Rᵤ, —C(O)ORᵤ and —C(O)NRᵥR_{w};

each Rₘ is independently H, (C₁-C₆)alkyl, (C₂-C₆)alkenyl, (C₂-C₆)alkynyl, (C₃-C₆)cycloalkyl, heterocycle, heteroaryl or aryl;

each Rₙ is independently (C₁-C₆)alkyl, (C₂-C₆)alkenyl, (C₂-C₆)alkynyl, (C₃-C₆)cycloalkyl, heterocycle, heteroaryl or aryl;

Rₒ and Rₚ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rₒ and Rₚ together with the nitrogen to which they are attached form a pyrrolidino, piperidino, piperazino, azetidino, morpholino, or thiomorpholino;

each R_{q} is independently (C₁-C₆)alkyl, (C₂-C₆)alkenyl, (C₂-C₆)alkynyl, (C₃-C₆)cycloalkyl, heterocycle, heteroaryl or aryl;

Rᵣ and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino;

each Rₜ is independently H, (C₁-C₆)alkyl, (C₂-C₆)alkenyl, (C₂-C₆) alkynyl or (C₃-C₆)cycloalkyl;

each Rᵤ is independently H, (C₁-C₆)alkyl, (C₂-C₆)alkenyl, (C₂-C₆)alkynyl, (C₃-C₆)cycloalkyl, heterocycle, heteroaryl or aryl;

Rᵥ and R_{w} are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵥ and R_{w} together with the nitrogen to which they are attached form a pyrrolidino, piperidino, piperazino, azetidino, morpholino, or thiomorpholino wherein any alkyl, alkenyl, alkynyl, cycloalkyl, heteroaryl, heterocycle, aryl pyrrolidino, piperidino, piperazino, azetidino, morpholino, or thiomorpholino of Rᵥ and R_{w} may be optionally substituted with one or more groups independently selected from CH₂OH, OH, NH₂ and CONH₂;

each Rₓ is independently H, (C₁-C₆)alkyl, (C₂-C₆)alkenyl, (C₂-C₆) alkynyl or (C₃-C₆)cycloalkyl;

each R_{y} is independently (C₁-C₆)alkyl, (C₂-C₆)alkenyl, (C₂-C₆)alkynyl, (C₃-C₆)cycloalkyl, heterocycle, heteroaryl or aryl wherein any alkyl, alkenyl, alkynyl, cycloalkyl, heterocycle, heteroaryl or aryl of R_{y} may be optionally substituted with one or more groups selected from ORᵤ and NRᵥR_{w};

each R_{z} is independently halo, heteroaryl, (C₁-C₆)alkyl, CN, —O(C₁-C₆)alkyl, NO₂, —C(O)OH, —(C₁-C₆)alkylNH₂, —(C₁-C₆)alkylOH, —NHC(O)(C₁-C₆)alkyl or —NHC(O)(C₁-C₆)alkylCN wherein heteroaryl is optionally substituted with —(C₁-C₆)alkylNH₂ or —(C₁-C₆)alkylOH;

each Rₐ₂ is independently H, (C₁-C₆)alkyl, (C₂-C₆)alkenyl, (C₂-C₆)alkynyl, (C₃-C₆)cycloalkyl, heterocycle, heteroaryl or aryl;

each R_{b2} is independently (C₁-C₆)alkyl, (C₂-C₆)alkenyl, (C₂-C₆)alkynyl, (C₃-C₆)cycloalkyl, heterocycle, heteroaryl or aryl;

R_{c2} and R_{d2} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c2} and R_{d2} together with the nitrogen to which they are attached form a pyrrolidino, piperidino, piperazino, azetidino, morpholino, or thiomorpholino; and

each Rₑ₂ is independently H, (C₁-C₆)alkyl, (C₂-C₆)alkenyl, (C₂-C₆) alkynyl or (C₃-C₆)cycloalkyl;

or a salt thereof.

A specific value for R_{g1} is (C₁-C₈)alkyl or (C₃-C₈)cycloalkyl, wherein any alkyl or cycloalkyl of R_{g1} may be optionally substituted with one or more oxo (C═O) or Rₖ groups.

Another specific value for R_{g1} is (C₄-C₈)alkyl or (C₄-C₈)cycloalkyl, wherein any alkyl or cycloalkyl of R_{g1} may be optionally substituted with one or more oxo (C═O) or Rₖ groups.

Another specific value for R_{g1} is (C₄-C₈)alkyl, wherein any alkyl of R_{g1} may be optionally substituted with one or more oxo (C═O) or Rₖ groups.

A specific value for Rₕ₁ is H or (C₁-C₆)alkyl, wherein any alkyl of Rₕ₁ may be optionally substituted with one or more oxo (C═O) or Rₖ groups.

Another specific value for R_{g1} is (C₁-C₈)alkyl, (C₃-C₈)cycloalkyl, aryl or heteroaryl.

Another specific value for R_{g1} is (C₄-C₈)alkyl, (C₄-C₈)cycloalkyl, aryl or heteroaryl.

Another specific value for R_{g1} is (C₄-C₈)alkyl or (C₄-C₈)cycloalkyl.

Another specific value for Rₕ₁ is H or (C₁-C₆)alkyl.

Another specific value for Rₕ₁ is H.

In another embodiment, the invention provides a specific group of compounds of formula I wherein:

A is furan optionally substituted with one or more (e.g. 1 or 2) R³ groups;

X is NH;

Y is heteroaryl;

R¹ is —C(O)NR_{g}Rₕ;

R² is —NR⁶R⁷;

each R³ is independently halo or (C₁-C₆)alkyl;

R⁶ is selected from (C₁-C₆)alkyl, (C₃-C₆)cycloalkyl, heteroaryl, heterocycle and aryl, and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino, wherein any alkyl, cycloalkyl, heteroaryl, heterocycle, aryl, pyrrolidino, piperidino, piperazino, azetidino, morpholino, or thiomorpholino of R⁶ and R⁷ may be optionally substituted with one or more (e.g. 1, 2 or 3) R¹¹ groups;

each R¹¹ is independently selected from (C₁-C₆)alkyl, heteroaryl and aryl, wherein alkyl, heteroaryl or aryl may be optionally substituted with one or more (e.g. 1, 2, 3, 4 or 5) groups selected from halo, R_{q}, OH, CN, —OR_{q}, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, CHO, —C(O)R_{q}, CO₂H, —C(O)OR_{q} and —C(O)NRᵣRₛ;

R_{g} is (C₁-C₆)alkyl or (C₃-C₆)cycloalkyl;

Rₕ is H

each R_{q} is independently (C₁-C₆)alkyl, (C₂-C₆)alkenyl, (C₂-C₆)alkynyl, (C₃-C₆)cycloalkyl, heterocycle, heteroaryl or aryl;

Rᵣ and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino; and

each Rₜ is independently H, (C₁-C₆)alkyl, (C₂-C₆)alkenyl, (C₂-C₆) alkynyl or (C₃-C₆)cycloalkyl;

or a salt thereof.

In another embodiment, the invention provides a specific group of compounds of formula I wherein:

A is furan;

X is NH;

Y is pyrazolyl;

R¹ is —C(O)NR_{g}Rₕ;

R² is —NR⁶R⁷;

R⁶ and R⁷ together with the nitrogen to which they are attached form a pyrrolidino substituted with one R¹¹ group;

R¹¹ is heteroaryl or —CH₂OH;

R_{g} is (C₁-C₆)alkyl or (C₃-C₆)cycloalkyl; and

Rₕ is H

or a salt thereof.

In another embodiment, the invention provides a specific group of compounds of formula I wherein:

A is furan;

X is NH;

Y is

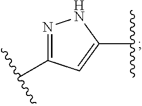

R¹ is —C(O)NR_{g}Rₕ;

R² is —NR⁶R⁷;

R⁶ and R⁷ together with the nitrogen to which they are attached form a pyrrolidino substituted with one R¹¹ group;

R¹¹ is pyridyl or —CH₂OH;

R_{g} is (C₁-C₆)alkyl or (C₃-C₆)cycloalkyl; and

Rₕ is H

or a salt thereof.

The invention also includes the compounds of formula I wherein one or more of the specific values and/or embodiments enumerated above are excluded from the compounds of formula I.

### Tautomers of Pyrazoles:

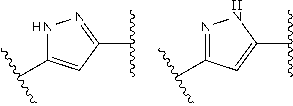

Pyrazoles may exhibit the isomeric forms referred as tautomers. Tautomers are isomeric forms of a compound that are in equilibrium with each other. The concentrations of the isomeric forms will depend on the environment in which the compound is found and may be different depending on if the compound is a solid or is in an organic or aqueous solution.

A wide variety of functional groups and other structures exhibit tautomerism and all tautomers of compounds of formula I are within the scope of the present invention.

Processes which can be used to prepare compounds of formula I and intermediates useful for preparing compounds of formula 1 are shown in Schemes 1-19.

### General Methods of Preparation of Invention Compounds:

Generally, heterocycles and hetereoaryls can be prepared from know methods as reported in the literature (a. Ring system handbook, published by American Chemical Society edition 1993 and subsequent supplements. b. The Chemistry of Heterocyclic Compounds; Weissberger, A., Ed.; Wiley: New York, 1962. c. Nesynov, E. P.; Grekov, A. P. The chemistry of 1,3,4-oxadiazole derivatives. Russ. Chem. Rev. 1964, 33, 508-515. d. Advances in Heterocyclic Chemistry; Katritzky, A. R., Boulton, A. J., Eds.; Academic Press: New York, 1966. e. In Comprehensive Heterocyclic Chemistry; Potts, K. T., Ed.; Pergamon Press: Oxford, 1984. f. Eloy, F. A review of the chemistry of 1,2,4-oxadiazoles. Fortschr. Chem. Forsch. 1965, 4, pp 807-876. g. Adv. Heterocycj. Chem. 1976. h. Comprehensive Heterocyclic Chemistry; Potts, K. T., Ed.; Pergamon Press: Oxford, 1984. i. Chem. Rev. 1961 61, 87-127. j. 1,2,4-Triazoles; John Wiley & Sons: New York, 1981; Vol 37). Some of the functional groups during the synthesis may need to be protected and subsequently deprotected. Examples of suitable protecting groups can be found in “Protective groups in organic synthesis” fourth edition edited by Greene and Wuts.

Scheme 1 outlines a general method which was used to synthesize compounds of formula I while Schemes 2 and 11 outline alternative methods which can be used to prepare compounds of formula I. Scheme 7 depicts a route to prepare intermediates which were used to prepare compounds of formula I; Schemes 3-6 and 8-10 depict alternative routes which can be used to prepare intermediates useful for preparing compounds of formula I. Schemes 12-19 depict methods that were used to prepare compounds of formula I. The intermediates prepared in Schemes 12-19 can also be useful for preparing additional compounds of formula I.

Representative compounds of formula 1 were prepared according to Scheme 1. A suitable furopyrimidine compound 1A with an appropriate leaving group (halo, sulfonates or other groups known in literature) was treated with an appropriately substituted amino compound in a suitable solvent such as alcohol in presence of base such as (and not restricted to) triethylamine to afford compound of general formula 1B. The second leaving group was displaced with an appropriately substituted amine either thermally or under microwave conditions using a base or amine and in a suitable solvent like dimethylformamide, dimethylacetamide or 1-methyl-2-pyrrolidinone (NMP), in presence or absence of a transition metal catalyst (known to one skilled in the art) to furnish a compound of formula 1.

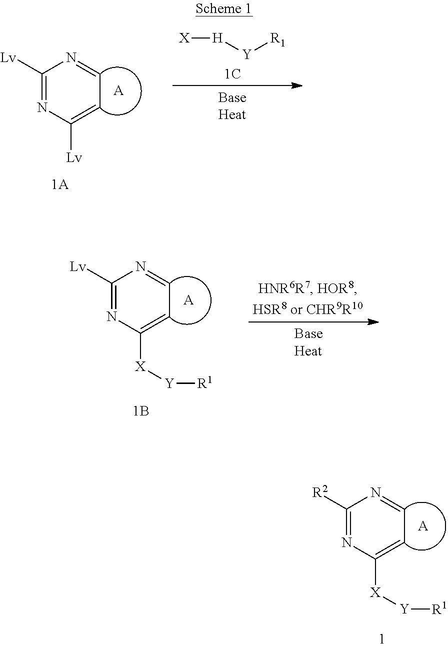

Scheme 2 depicts a general methodology which can be used to obtain a compound of formula 1. Reaction of guanidine 2A with an appropriately substituted alkyl 3-aminofuran-2-carboxylate, 2-aminofuran-3-carboxylate or 4-aminofuran-3-carboxylate 2B can afford an appropriately substituted hydroxy-furo[3,2-d]pyrimidine, hydroxy-furo[2,3-d]pyrimidine or hydroxyl-furo[3,4-d]pyrimidine 2D. The hydroxyl on pyrimidine 2D can be converted to a halo pyrimidine 2E using phosphorous oxyhalides. The halo group on 2E may be displaced with an appropriately substituted amine either thermally or under microwave conditions using a base or amine and in a suitable solvent like dimethylformamide, dimethylacetamide or 1-methyl-2-pyrrolidinone (NMP), in the presence or absence of a transition metal catalyst (known to one skilled in the art) to furnish compound of formula 1. Similarly guanidine 2A can be reacted with an appropriately substituted 3-aminofuran-2-carbonitrile, 2-aminofuran-3-carbonitrile or 4-aminofuran-3-carbonitrile 2C to furnish an appropriately substituted amino-furo[3,2-d]pyrimidine, amino-furo[2,3-d]pyrimidine or amino-furo[3,4-d]pyrimidine 2F. Title compound 1 can be obtained by cross coupling reactions involving the amine 2F and a suitable leaving group on 2G using a transition-metal catalyst and conditions known in literature (for literature example of transition-metal catalyzed cross coupling reactions, see: a. Y. Monguchi, et al., Advanced Synthesis & Catalysis, 2008, 350, 2767-2777; b. T. Watanabe, et al., Chemical Communications (Cambridge, United Kingdom), 2007, 43, 4516-4518; c. M. Kienle, et al., European Journal of Organic Chemistry, 2007, 25, 4166-4176; d. H.-Z. Zhang, et al., Bioorganic & Medicinal Chemistry, 2008, 16, 222-231; e. J. P. Schulte II, et al., Synlett. 2007, 15, 2331-2336; f. C. Yang, et al., CN 101475493 A 20090708; g. S.-E. Park, et al., Synthesis, 2009, 5, 815-823; h. L. Rout, et al., Advanced Synthesis & Catalysis, 2008, 350, 395-398; i. H. Huang, et al., Journal of Organic Chemistry, 2008, 73, 6037-6040. j. J. Li, et al., Journal of Organometallic Chemistry, 2007, 692, 3732-3742. k. C. Chen, et al., Journal of Organic Chemistry, 2007, 72, 6324-6327; l. L. Rout, et al., Organic Letters, 2007, 9, 3397-3399; m. C. Xu, et al., Tetrahedron Letters, 2007, 48, 1619-1623; n. X. Xie, et al., Journal of Organic Chemistry, 2006, 71, 6522-6529; o. S. Harkal, et al., Advanced Synthesis & Catalysis, 2004, 346, 1742-1748. p. Yuki Gosei Kagaku Kyokaishi., Synlett, 2005, 63, 80-81; q. L. J. Goossen, et al., Synlett., 2005, 2, 275-278; r. F. Rataboul, et al., Chemistry—A European Journal, 2004, 10, 2983-2990; s. A. S. Gajare, et al., Chemical Communications (Cambridge, United Kingdom), 2004, 17, 1994-1995; t. C. Desmarets, et al., Journal of Organic Chemistry, 2002, 67, 3029-3036; u. C. F. Allen, Chemical Reviews (Washington, D.C., United States), 1959, 59, 983-1030; v. et al., Journal of Organic Chemistry, 2001, 66, 1403-1412; w. N. Kataoka, et al., Journal of Organic Chemistry, 2002, 67, 5553-5566; x. J. P. Wolfe, et al., J. Am. Chem. Soc., 1996, 118, 7215-7216. y. S. Wagaw, et al., J. Am. Chem. Soc., 1997, 119, 8451-8458; z. J.-F. Marcoux, S et al., J. Org. Chem., 1997, 62, 1568-1569; aa. J. P. Wolfe, S et al., J. Org. Chem., 1997, 62, 6066-6068; ab. J. P. Wolfe, et al., J. Am. Chem. Soc. 1997, 119, 6054-6058; ac. R. Kuwano, et al., J. Org. Chem. 2002, 67, 6479-6486).

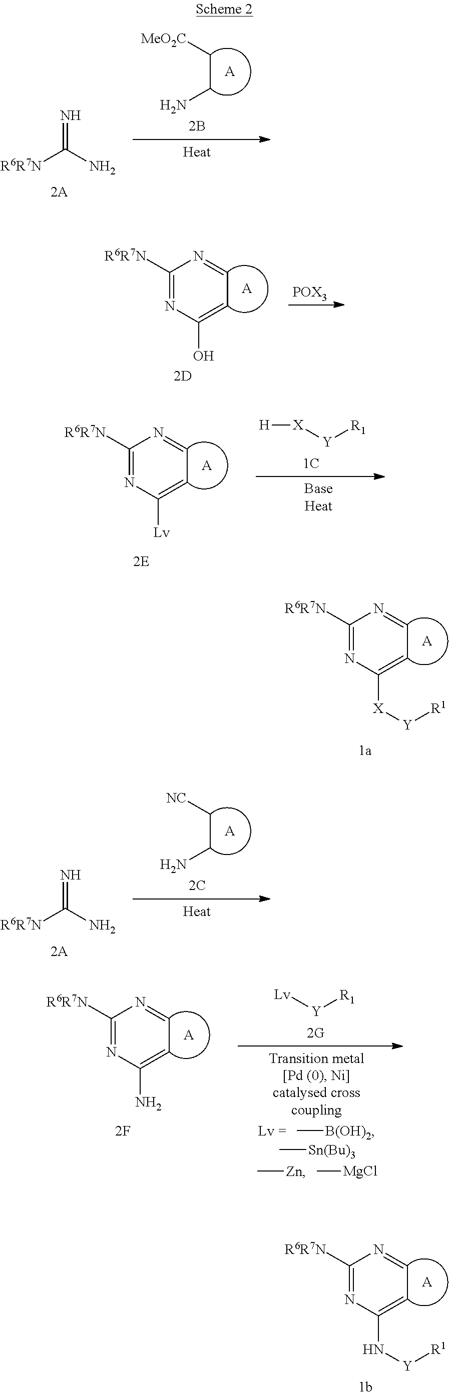

Appropriately substituted hydroxy-furo[3,2-d]pyrimidines, hydroxy-furo[2,3-d]pyrimidines or hydroxyl-furo[3,4-d]pyrimidines 2D can also be obtained by the method depicted in Scheme 3. The cyano group can be introduced on amine 3A using cyanogen bromide or other known methods in the literature to furnish compound 3B. Treatment of nitrile 3B with alcohol under acidic condition can afford imidate 3C. Reaction of imidate 3C with an appropriately substituted alkyl 3-aminofuran-2-carboxylate, 2-aminofuran-3-carboxylate or 4-aminofuran-3-carboxylate 2B can provide an appropriately substituted hydroxy-furo[3,2-d]pyrimidine, hydroxy-furo[2,3-d]pyrimidine or hydroxyl-furo[3,4-d]pyrimidine 2D.

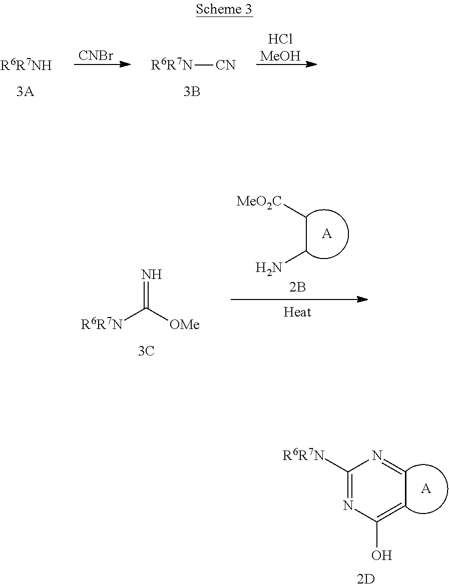

Scheme 4 depicts methods which can be used to prepare intermediates 4B and 4D. Oxidation of the nitrile of an appropriately substituted 3-aminofuran-2-carbonitrile, 2-aminofuran-3-carbonitrile or 4-aminofuran-3-carbonitrile 2C can provide amide 4A. The amide 4A can be cyclized to the appropriately substituted hydroxy-furo[3,2-d]pyrimidine, hydroxy-furo[2,3-d]pyrimidine or hydroxyl-furo[3,4-d]pyrimidine guanine 4B using the conditions as depicted on Scheme 4. The hydroxyl of compound 4B can be converted to an appropriate leaving group, usually a halide to give compound 4C. Diazotization followed by halogenation gives the compound 4D. Similarly, an appropriately substituted alkyl 3-aminofuran-2-carboxylate, 2-aminofuran-3-carboxylate or 4-aminofuran-3-carboxylate 2B can be cyclized to the appropriately substituted hydroxy-furo[3,2-d]pyrimidine, hydroxy-furo[2,3-d]pyrimidine or hydroxyl-furo[3,4-d]pyrimidine guanine 4B using the conditions as depicted on Scheme 4.

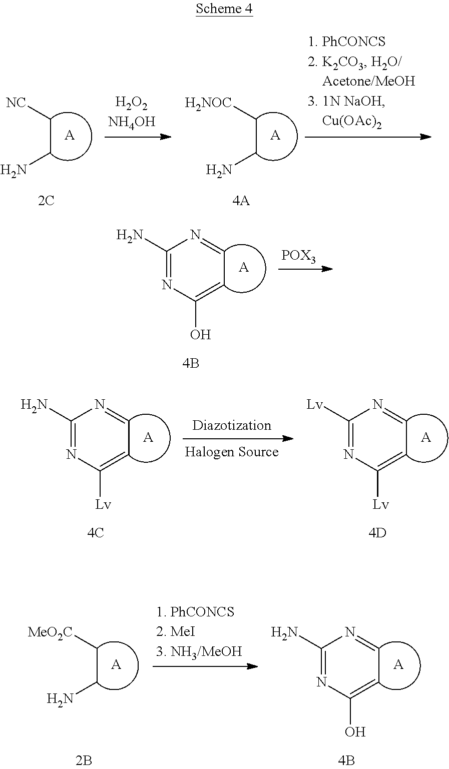

Scheme 5 illustrates a methodology that can be used for the preparation of dihalo furo[3,2-d]pyrimidine compounds 5F. The starting material 3-iodofuran-2-carboxylic acid 5A can be prepared by literature procedures (a. T. G. Hamill, et al., Journal of Labelled Compounds & Radiopharmaceuticals, 2001, 44, 61-72; b. J.-M. Duffault, et al., Synthetic Communications, 1998, 28, 2467-2481; c. M. Takahashi, et al., Heterocycles, 1993, 36, 1867-82; d. R. Sornay, et al., Bulletin de la Societe Chimique de France, 1971, 3, 990-1000). Compound 5A can be reacted with sodium azide to give compound 5B, which upon reduction can generate compound 5C. Further cyclization of compound 5C to 5D can be achieved using guanidine. The hydroxyl of compound 5D can be converted to an appropriate leaving group such as a halide to give compound 5E. Diazotization followed by halogenation can provide compound 5F.

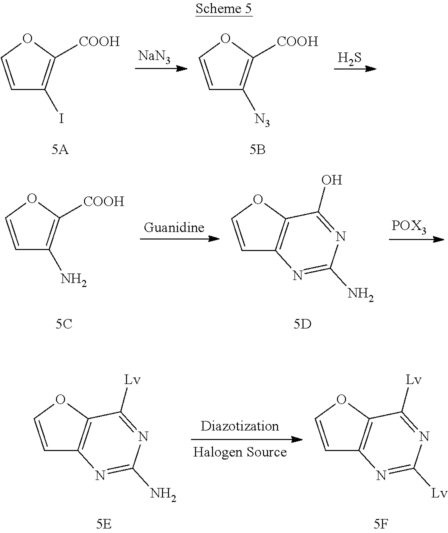

Alternatively compound 5D can be prepared from methyl 3-nitrofuran-2-carboxylate (6A) as outlined in Scheme 6. The starting material methyl 3-nitrofuran-2-carboxylate can be prepared by literature methods (S. A. Shackelford, et al., Journal of Organic Chemistry, 2003, 68, 267-275). Reduction of the nitro on 6A to amine 6B followed by cyclization using methylated thiourea can provide furo[3,2-d]pyrimidine 5D. Reaction conditions for conversion of amine 6B to guanine furo[3,2-d]pyrimidine 5D can be found in the literature (a. R. Nigel, et al., Eur. Pat. Appl., 2009, 19 pp, EP 2020412 A1 20090204; b. Y. S. Babu, P. et al., PCT Int. Appl., 2006, 152 pp, WO 2006050161).

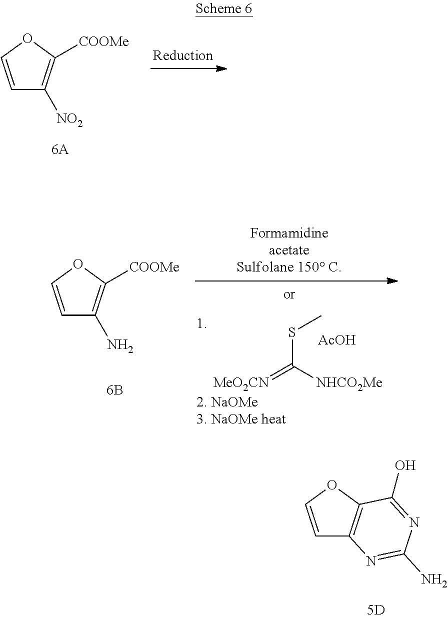

Scheme 7 outlines a method that was used to prepare compound 2,4-halofuro[3,2-d]pyrimidine 7K as well as some alternative preparations. The starting material 3-halo-acrylonitrile 7A (J. Org. Chem., 1992, 57, 708-713) can be treated with the sodium salt of 2-hydroxyacetonitrile to give compound 7B which is analogous to the reaction described in J. Med. Chem., 2000, 43, 4288-4312. 3-Hydroxypropenenitrile (J. Org. Chem., 1991, 56, 970-975) on treatment with haloacetonitrile also generates 7B. Compound 7B can be treated with strong base, such as lithium-N,N-diisopropylamide or sodium ethoxide, to generate compound 7C (Tetrahedron Lett., 1986, 27, 815-818). The cyano group on compound 7C can be converted to give ester compound 7E. Similarly, treatment of compound 7A with bromodiethylmalonate can furnish compound 7D which on base cyclization yields ester compound 7E. Alternatively compound 7E was prepared from 3-furoic acid 7F. Curtius rearrangement of compound 7F using diphenylphosphoryl azide in presence of base and tert-butanol as solvent gave the boc protected amino compound 7G. The methoxy carbonyl group was introduced using base and dimethyl carbonate to furnish compound 7H. Hydrolysis of Boc group on compound 7H gave the desired compound 7E. Reaction of compound 7E with chlorosulfonyl isocyanate provided the urea compound 7I, which was cyclized under basic conditions to dihydroxy furo[3,2-d]pyrimidine 7J. Reaction of compound 7J with POCl₃ gave compound 7K (alternative phosphorous oxyhalides can provide other dihalo furo[3,2-d]pyrimidines 1A). Alternatively dihydroxy furo[3,2-d]pyrimidine 7J can be obtained from 7E using benzoyl isocyanate followed by hydrolysis with a base.

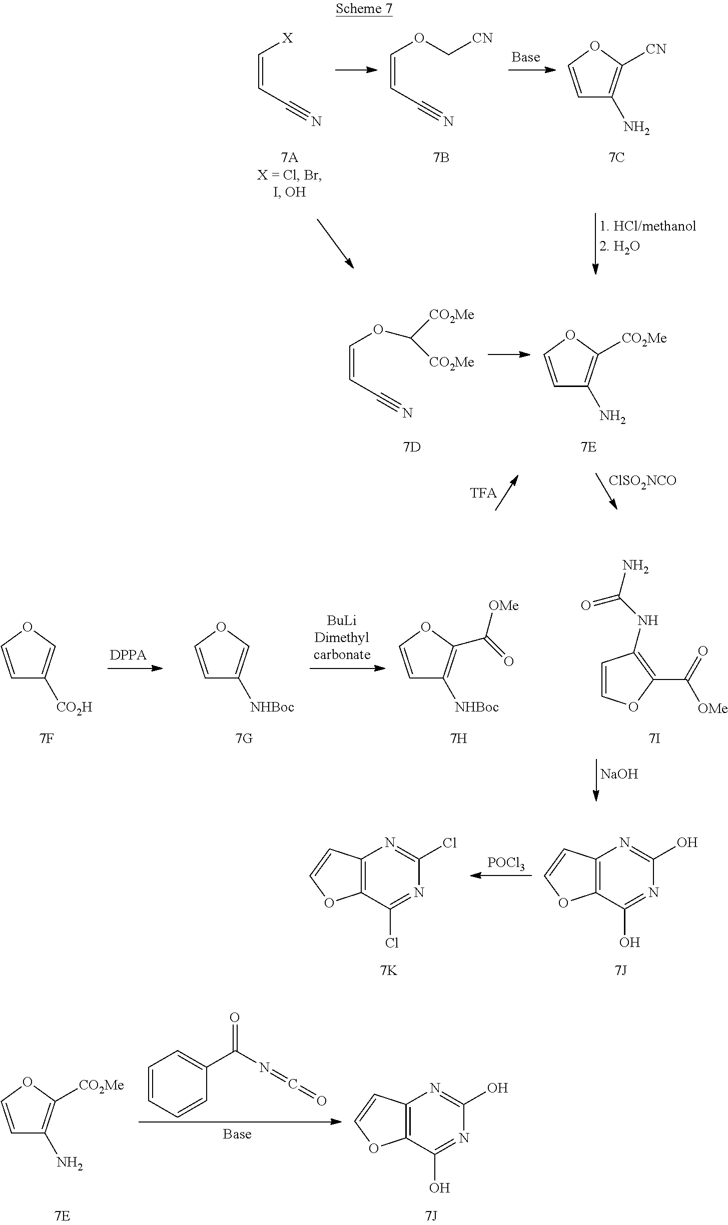

Scheme 8 illustrates a preparation of furo[2,3-d]pyrimidine types of compounds. Treatment of 1,4-dioxane-2,5-diol 8A with malononitrile under basic conditions can provide 2-aminofuran-3-carbonitrile compound 8B. Compound 8B can be converted to methyl 2-aminofuran-3-carboxylate 8C by known procedures which include conversion of ester via imidate followed by hydrolysis of the imidate to ester 8C. The rest of the steps for the conversion of compound 8C to 8F are similar as shown in Scheme 7 for the conversion of 7E to 7K to provide dihalo furo[2,3-d]pyrimidine type compound 8F. Compound 8B can be cyclized with guanidine or other methods to guanine 8G followed by diazotization and halogenation of 8G to provide compound 8F.

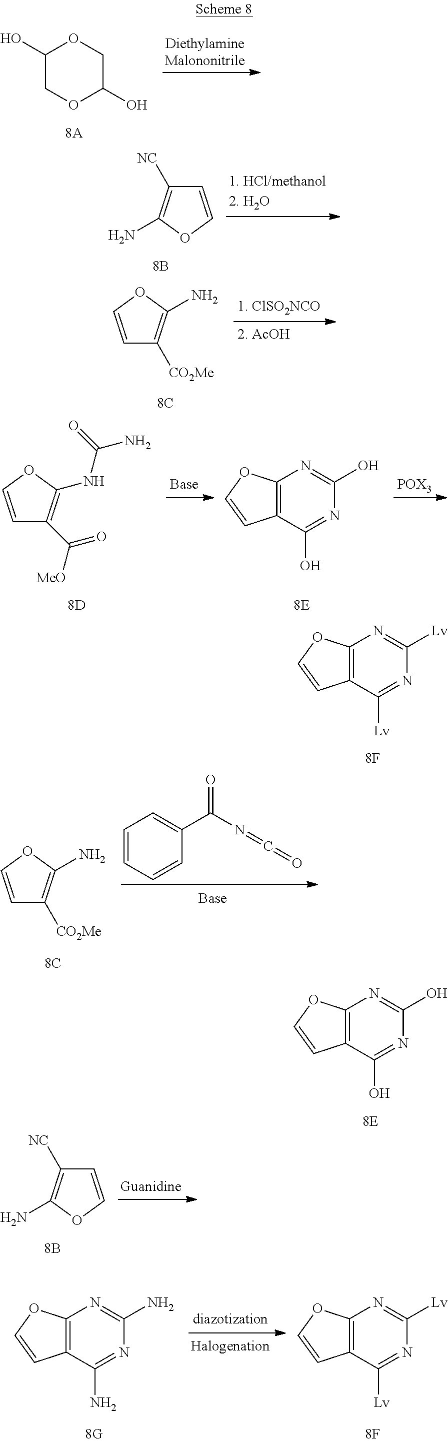

Scheme 9 illustrates an alternate preparation for furo[2,3-d]pyrimidine intermediates 8F. 2,4,6-Halo pyrimidines 9A can be reacted with 2,2-diethoxyethanol in presence of base like sodium hydride to obtain compound 9B which can be cyclized with phosphoric acid to furnish the desired furo[2,3-d]pyrimidine type compound 8F.

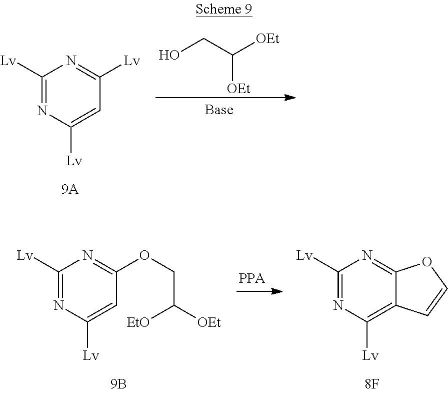

Scheme 10 depicts a method for preparation of furo[3,4-d]pyrimidine intermediates (10G). Commercially available diethyl furan-3,4-dicarboxylate or dimethyl furan-3,4-dicarboxylate can be hydrolyzed to monoester 10B using procedures described in the literature (a. K. Yabu, et al., Tetrahedron Letters, 2002, 43, 2923-2926; b. D. J. Ager, et al., Synthetic Communications, 1995, 25, 739-42; c. W. Loesel, et al., Ger. Offen., 1983, 21 pp, DE 3143876; d. S. P. Tanis, Tetrahedron Letters, 1982, 23, 3115-18; e. S. Kakimoto, et al., Hokkaido Daigaku Men'eki Kagaku Kenkyusho Kiyo, 1976, 36, 13-16; f. M. R. Boyd, et al., Synthesis, 1971, 10, 545-6; g. R. R. Doyle, et al., Journal of the Scientific Laboratories, Denison University, 1971, 52 (Art. 1-5), 5-8; h. K. Galuszko, et al., Univ. Warsaw, Roczniki Chemii, 1964, 38, 511-13; i. M. R. Boyd, et al. Nature (London), New Biology, 1972, 236, 158-9; J. R. Andrisano, et al., Gazzetta Chimica Italiana, 1953, 83, 340-6).

Reaction of compound 10B with excess hydrazine hydrate can provide hydrazide 10C. Hydrazide 10C can be converted to acylazide 1°D using aqueous nitrous acid. Heating a solution of acylazide in an appropriate solvent can provide 1H-furo[3,4-d][1,3]oxazine-2,4-dione 10E (references for preparation of 1H-furo[3,4-d][1,3]oxazine-2,4-dione 10E include: a. C. Zhan, et al., 2008, 23 pp, CN 101293909; b. T. O. Olagbemiro, Bulletin des Societes Chimiques Belges, 1981, 90, 1067-72. c. J. B. Press, et al., Journal of Organic Chemistry, 1981, 46, 3853-6. Compound 10E can be converted to furo[3,4-d]pyrimidine-2,4(1H,3H)-dione 10F by reacting 10E with ammonia followed by cyclization using carbonyl diimidazole (references for preparation of furo[3,4-d]pyrimidine-2,4(1H,3H)-dione 10F include: a. S. Butini, et al., Journal of Medicinal Chemistry, 2008, 51, 6614-6618; b. R. G. Jones, Journal of Organic Chemistry, 1960, 25, 956-9. Reaction of compound 10F with phosphorous oxyhalide can give dihalo furo[3,4-d]pyrimidine 10G.

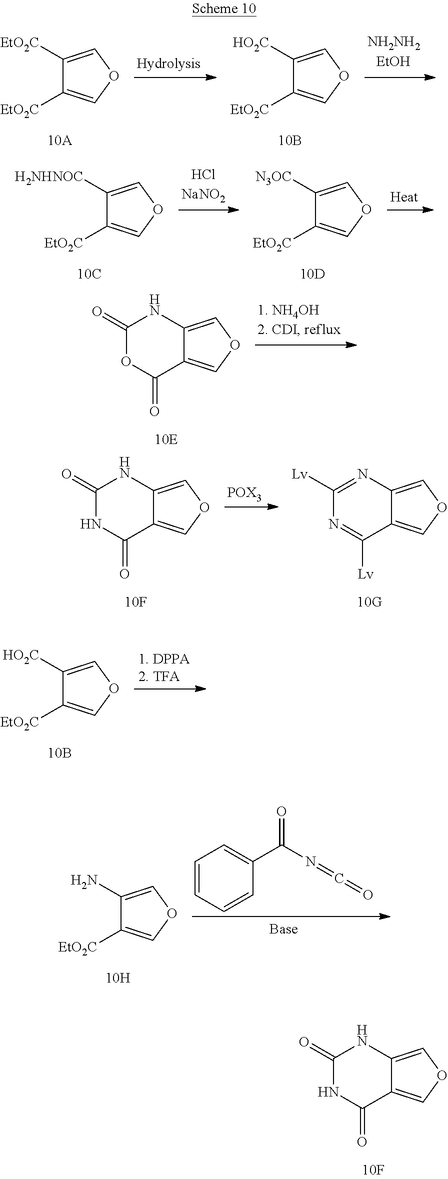

Compounds of formula 11G can be prepared according to Scheme 11. Guanylation of amine 11A with amino(imino)methanesulfonic acid 11B can provide the guanidine of formula 2A. Condensation of guanidine 2A with dialkylmalonate can provide dihydroxy pyrimidine 11C. The hydroxyl on pyrimidine 11C can be converted to a halo pyrimidine 11D using a phosphorous oxyhalide. Reaction of dibromo pyrimidine 11D with 2,2-diethoxyethanol can afford compound 11E which can be cyclized to bromo-furo[2,3-d]pyrimidine 11F using PPA or other acids. The halo group on 11F may be displaced with an appropriately substituted amine either thermally or under microwave conditions using a base or amine and in a suitable solvent like dimethylformamide, dimethylacetamide or 1-methyl-2-pyrrolidinone (NMP), in presence or absence of a transition metal catalyst (known to one skilled in the art) to furnish compound of formula 11G.

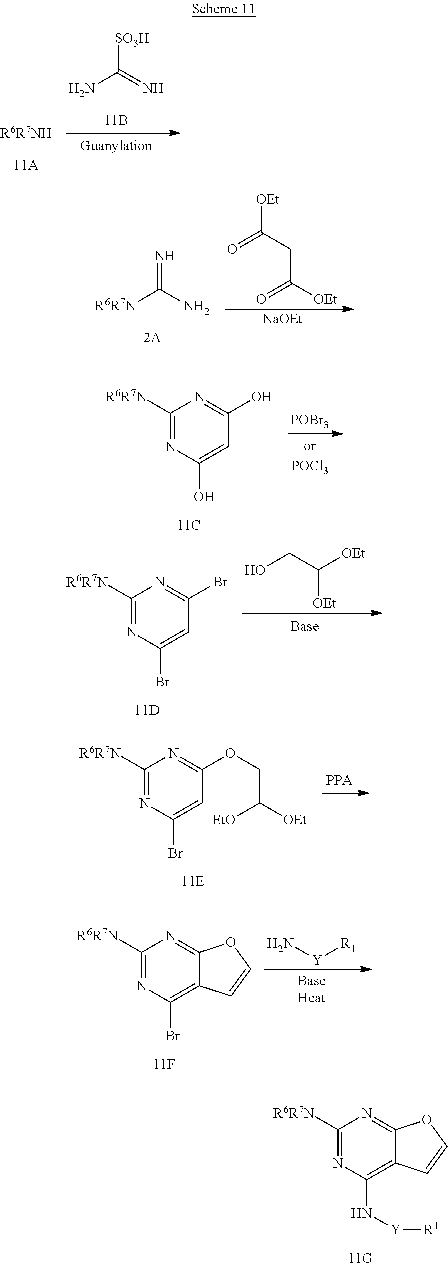

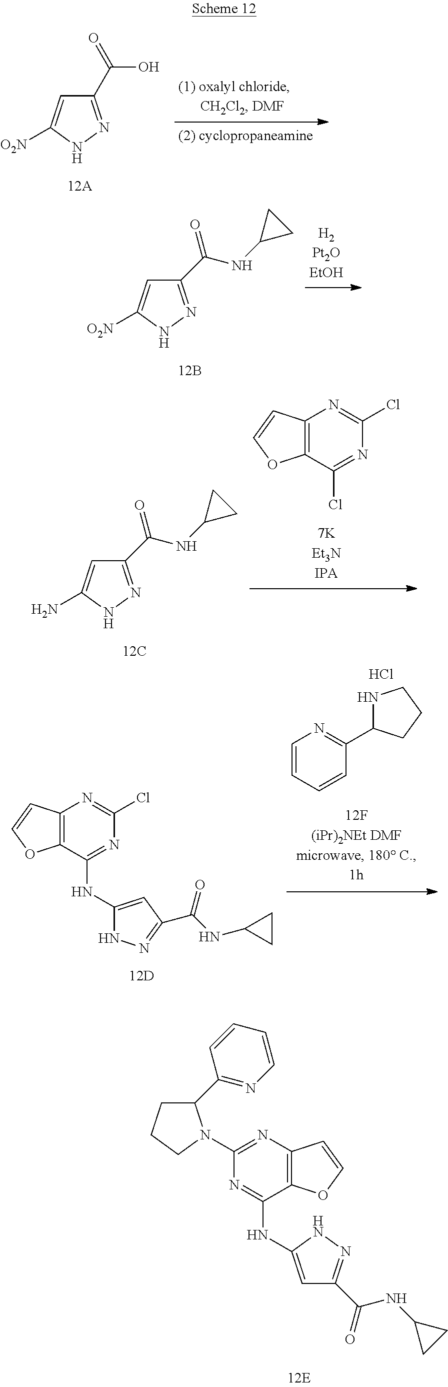

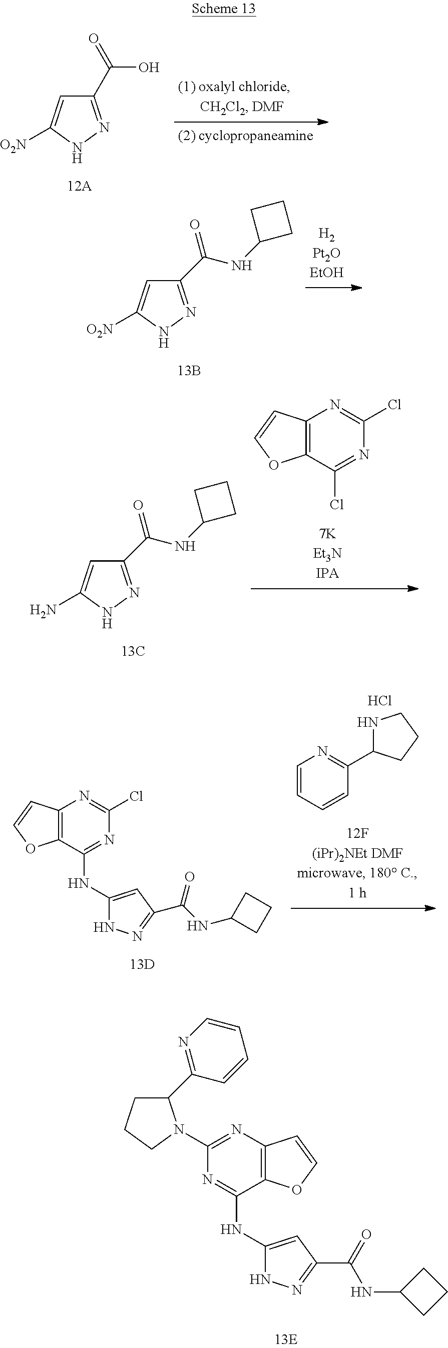

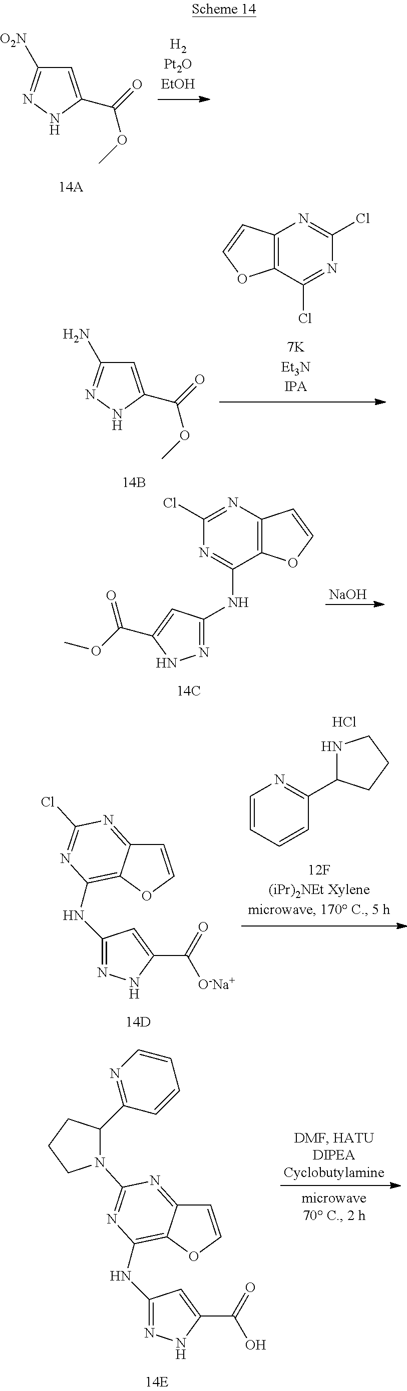

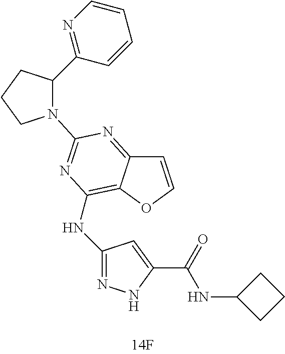

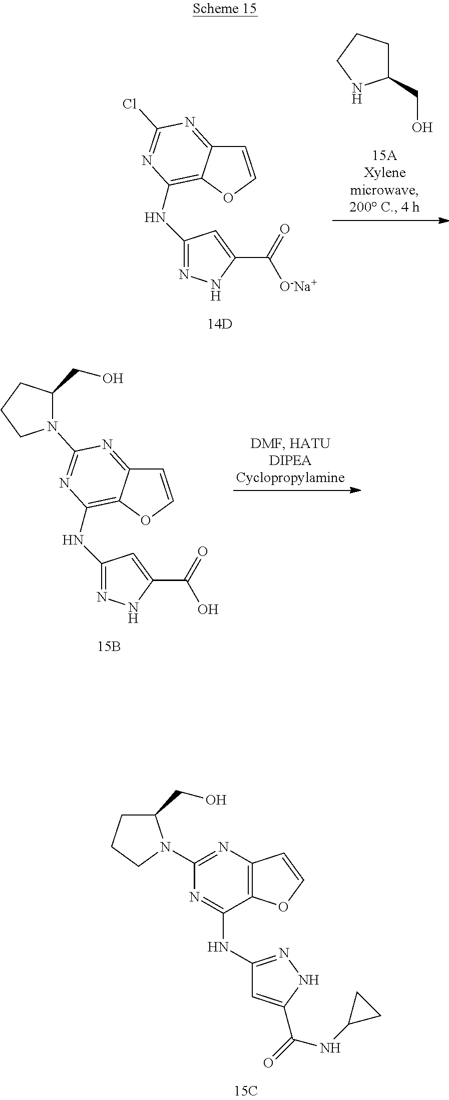

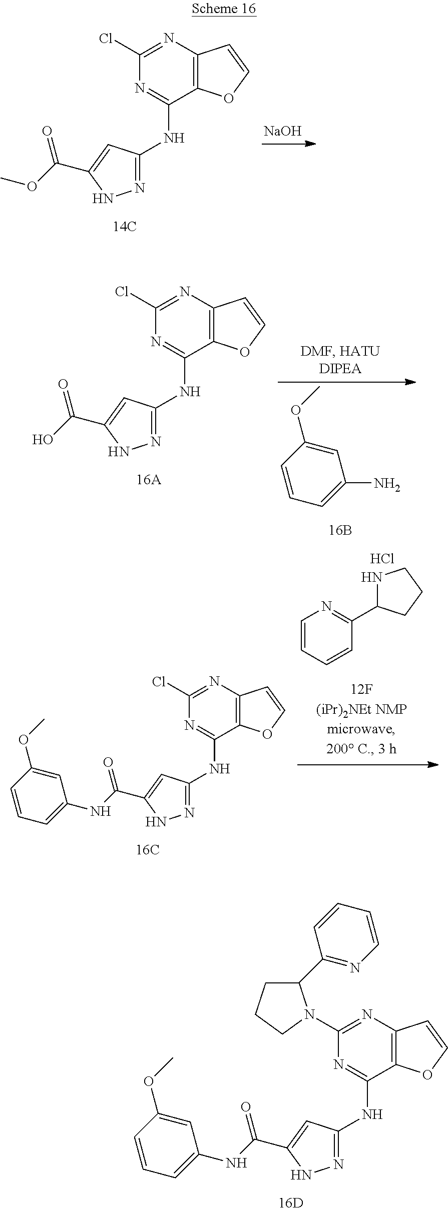

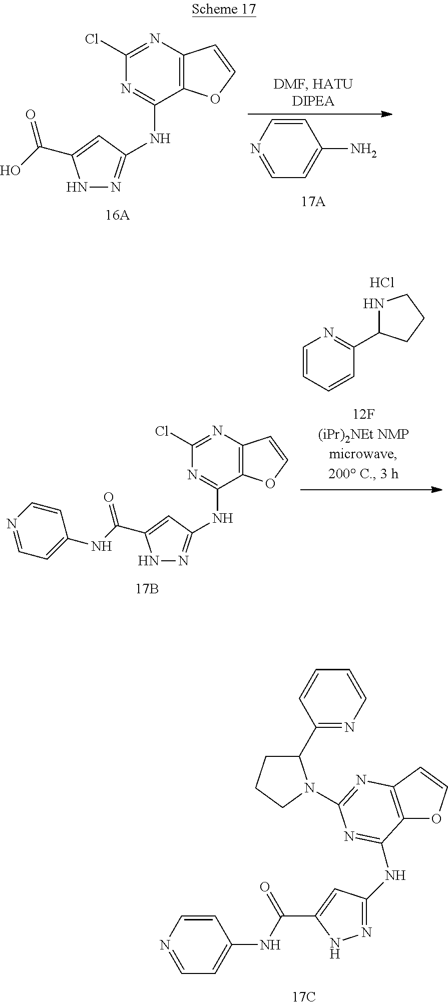

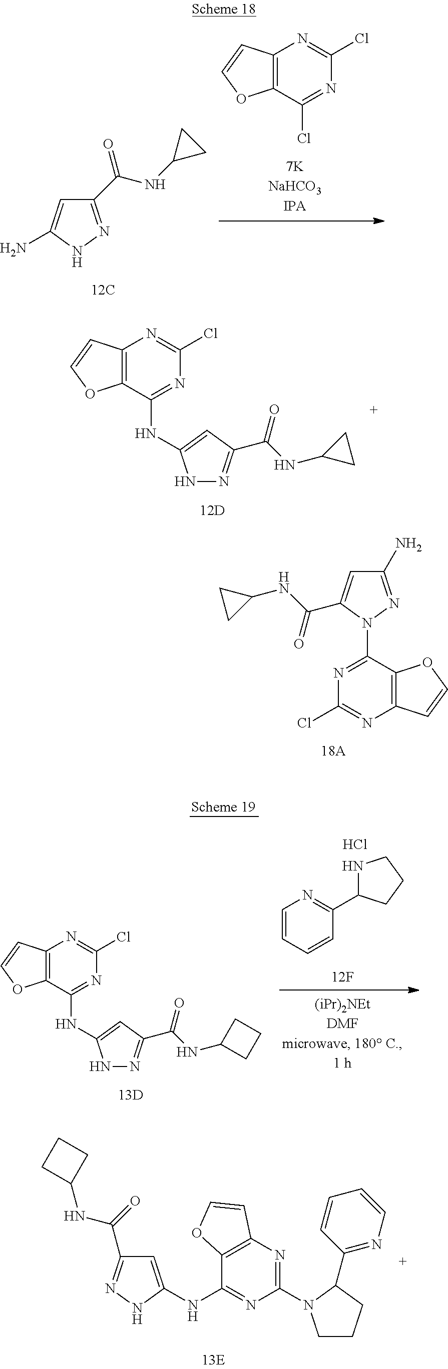

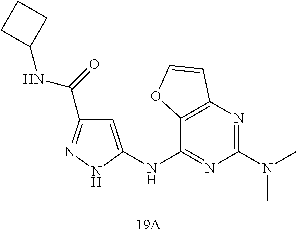

A compound of formula I can be prepared by displacing a leaving group from a compound of formula 1B:

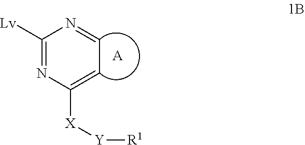

to provide the corresponding compound of formula I, for example by displacing the leaving group of formula 1B with a nucleophile (e.g. an amine, alcohol, thiol or carbanion) to provide a compound of formula I. Thus, the intermediate of formula 1B is useful for preparing a compound of formula I.

A compound of formula I can be prepared by displacing a leaving group from a compound of formula 1B′:

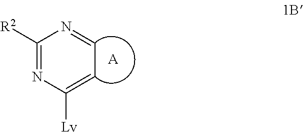

to provide the corresponding compound of formula I, for example by displacing the leaving group of formula 1B′ with a nucleophile (e.g. an amine, alcohol, thiol or carbanion) to provide a compound of formula I. Thus, the intermediate of formula 1B′ is useful for preparing a compound of formula I.

Accordingly, the invention provides a method:
a) for preparing a compound of formula I comprising treating a corresponding compound of formula 1B with an appropriate nucleophile (e.g. an amine, alcohol, thiol or carbanion) to provide the compound of formula I.
b) for preparing a compound of formula I comprising treating a corresponding compound of formula 1B′ with an appropriate nucleophile (e.g. an amine, alcohol, thiol or carbanion) to provide the compound of formula I.
c) for preparing a compound of formula I comprising deprotecting a corresponding compound bearing one or more protecting groups to provide the compound of formula I.
d) for preparing a salt of a compound of formula I comprising treating a corresponding compound of formula I with an acid (e.g. an organic acid or inorganic acid) or base (e.g. an alkali base or alkaline base) to provide the salt of the compound of formula I.

In one embodiment, the invention provides a method for preparing a salt of a compound of formula I, comprising reacting the compound of formula I with an acid under conditions suitable to provide the salt.

In one embodiment, the invention provides a method for preparing a pharmaceutical composition comprising a compound of formula I, or a pharmaceutically acceptable salt thereof, in combination with a pharmaceutically acceptable diluent or carrier, comprising combining the compound of formula I, or the pharmaceutically acceptable salt thereof, with the pharmaceutically acceptable diluent or carrier to provide the pharmaceutical composition.

The compounds of formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for immunosuppression. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to suppress an immune response in the animal.

Compounds of the invention may also be useful in the treatment of other diseases, conditions or disorders associated with the function of a kinase such as a Janus kinase (e.g. JAK1, JAK2 or TYK2) including the pathological activation of a kinase such as a Janus kinase (e.g. JAK1, JAK2 or TYK2). Accordingly, in one embodiment the invention provides a compound of formula I for the treatment of a kinase such as a Janus kinase (e.g. JAK1, JAK2 or TYK2) related disease, condition or disorder.

The ability of a compound of the invention to bind to JAK3 may be determined using pharmacological models which are well known to the art, or using Test A described below.

### Test A.

Inhibition constants (IC₅₀s) were determined against JAK3 (JH1domain-catalytic) kinase and other members of the JAK family. Assays were performed as described in Fabian et al. (2005) Nature Biotechnology, vol. 23, p. 329 and in Karaman et al. (2008) Nature Biotechnology, vol. 26, p. 127. Inhibition constants were determined using 11 point dose response curves which were performed in triplicate. Table 1 shown below lists compounds of the invention and their respective IC₅₀ values.

The ability of a compound of the invention to provide an immunomodulatory effect can also be determined using pharmacological models which are well known to the art. The ability of a compound of the invention to provide an anti-cancer effect can also be determined using pharmacological models which are well known to the art.

The invention will now be illustrated by the following non-limiting Examples of the preparation of compounds of the invention and intermediates.

### Example 1

### N-cyclopropyl-5-(2-(2-(pyridin-2-yl)pyrrolidin-1-yl)furo[3,2-d]pyrimidin-4-ylamino)-1H-pyrazole-3-carboxamide (12E)

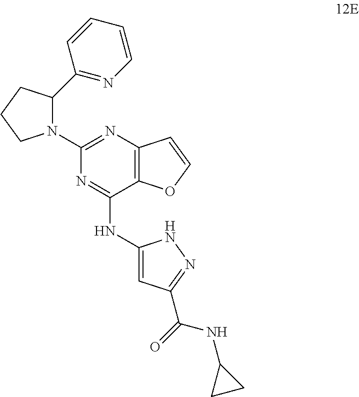

To a solution of 5-(2-chlorofuro[3,2-d]pyrimidin-4-ylamino)-N-cyclopropyl-1H-pyrazole-3-carboxamide (12D) (0.115 g, 0.36 mmol) in DMF (0.5 mL) was added 2-(pyrrolidin-2-yl)pyridine Hydrochloride (12F) (0.067 g, 0.36 mmol), DIPEA (0.157 mL, 0.9 mmol) and heated in a microwave for 1 h at 180° C. The reaction mixture was concentrated in vacuo and purified by flash column chromatography [silica gel, eluting with eluting with 0-100% (9:1) ethyl acetate/methanol in hexanes] to furnish N-cyclopropyl-5-(2-(2-(pyridin-2-yl)pyrrolidin-1-yl)furo[3,2-d]pyrimidin-4-ylamino)-1H-pyrazole-3-carboxamide (12E) (0.038 g, 25%) as an off-white solid; mp 238.4° C.; ¹H NMR (300 MHz, MeOD) δ 8.62-8.42 (m, 1H), 7.85 (s, 1H), 7.78-7.63 (m, 1H), 7.22 (m, 2H), 6.66 (s, 1H), 6.30 (bs, 1H), 5.51-5.25 (m, 1H), 4.08-3.95 (m, 1H), 3.88-3.72 (m, 1H), 2.97-2.75 (m, 1H), 2.63-2.41 (m, 1H), 2.08 (s, 3H), 0.84 (s, 4H); ¹H NMR (300 MHz, DMSO/D₂O) δ 8.61-8.39 (m, 1H), 8.20-7.94 (m, 1H), 7.76-7.60 (m, 1H), 7.20 (m, 3H), 6.88-6.62 (m, 1H), 5.42-5.16 (m, 1H), 4.02-3.82 (m, 1H), 3.78-3.61 (m, 1H), 2.95-2.75 (m, 1H), 2.47-2.31 (m, 1H), 1.97 (s, 3H), 0.64 (s, 4H). MS (ES+) 431.9 (M+1), 883.15 (2M+Na), (ES−) 429.19 (M−1), 465.20 (M+Cl).

### Preparation of intermediate compound 5-(2-chlorofuro[3,2-d]pyrimidin-4-ylamino)-N-cyclopropyl-1H-pyrazole-3-carboxamide (12D)

### Step 1:

A solution of 2M oxalyl chloride (in dichloromethane, 7 mL, 14 mmol) was added to a suspension of 5-nitro-3-pyrazocarboxylic acid (12A) (1.10 g, 7 mmol) in dichloromethane (25 mL) and THF (0.7 mL) at 0° C. One drop of DMF was added to the reaction mixture and stirred at room temperature for 3 h. The reaction mixture was evaporated in vacuum to dryness and the residue obtained was dissolved in dichloromethane (35 mL). To the solution was added a mixture of cyclopropaneamine (0.6 mL, 8.5 mmol), pyridine (1.13 mL), and dichloromethane (2 mL) over a period of 10 min. After stirring at room temperature overnight, the reaction mixture was concentrated in vacuum to dryness and the residue obtained was purified by flash column chromatography (silica gel, eluting with hexanes/ethyl acetate 0 to 100%) to furnish N-cyclopropyl-5-nitro-1H-pyrazole-3-carboxamide (12B) (0.93 g, 68%) as an off-white solid; mp 206.5° C.; ¹H NMR (300 MHz, DMSO) δ 14.80 (s, 1H), 8.76 (d, J=3.8 Hz, 1H), 7.56 (s, 1H), 2.96-2.70 (m, 1H), 0.75 (td, J=4.7, 7.1 Hz, 2H), 0.58 (dt, J=4.5, 7.3 Hz, 2H).

### Step 2:

To a solution of N-cyclopropyl-5-nitro-1H-pyrazole-3-carboxamide (12B) (0.9 g, 4.6 mmol) in ethanol (20 mL) was added platinum oxide (125 mg) and hydrogenated at 60 psi for 3 h. The catalyst was removed by filtration through a pad of celite and the filtrate was concentrated in vacuo to give 5-amino-N-cyclopropyl-1H-pyrazole-3-carboxamide (12C) (0.8 g, 100%) as a olive solid; mp >246° C.; ¹H NMR (300 MHz, DMSO) δ 12.20-11.68 (bs, 1H), 8.39-7.62 (bs, 1H), 6.12-5.41 (bs, 1H), 5.32-4.49 (bs, 2H), 2.74 (m, 1H), 0.64 (m, 2H), 0.54 (m, 2H).

### Step 3:

To a solution of 2,4-dichlorofuro[3,2-d]pyrimidine (7K) (0.19 g, 1 mmol) in isopropanol (10 mL) was added triethylamine (0.21 mL, 1.5 mmol), 5-amino-N-cyclopropyl-1H-pyrazole-3-carboxamide (12C) (0.2 g, 1.2 mmol) and heated at reflux for 48 h. The reaction mixture was concentrated in vacuo to dryness and the residue obtained was purified by flash column chromatography [silica gel 24 g, eluting with 0-100% (9:1) ethyl acetate/methanol in hexanes] to furnish 5-(2-chlorofuro[3,2-d]pyrimidin-4-ylamino)-N-cyclopropyl-1H-pyrazole-3-carboxamide (12D) (0.12 g, 38%) as a light yellow solid; mp 246.8° C. ¹H NMR (300 MHz, DMSO) δ 13.26 (s, 1H), 10.77 (s, 1H), 8.60 (d, J=3.9 Hz, 1H), 8.37 (d, J=2.1 Hz, 1H), 7.09 (s, 1H), 7.04 (d, J=2.1 Hz, 1H), 2.82 (d, J=3.9 Hz, 1H), 0.76-0.68 (m, 2H), 0.60 (m, 2H). MS (ES−) 316.91 (M−1).

### Preparation of intermediate compound 2,4-dichlorofuro[3,2-d]pyrimidine (7K)

### Step-1:

Diphenyl phosphoryl azide (50 g) was added dropwise over 45 min to a solution of 3-furoic acid 7F (54.4 g), triethylamine (108 mL) and t-BuOH (78 mL) in toluene (800 mL). The solution was heated at reflux for 6 h and then at room temperature overnight. The reaction was quenched with water (1000 mL) and the resulting solution extracted with ethyl acetate (3×1000 mL). The combined organic phases were washed with water (800 mL), brine (800 mL), decolorized with activated charcoal, dried with MgSO₄, filtered and concentrated in vacuo to furnish a brown semisolid which was treated with CH₂Cl₂ (300 mL), and hexanes (600 mL). The solid was filtered to give tert-butyl furan-3-ylcarbamate (7G) (48.7 g, 60%) as white needles; mp 136.5° C.; ¹H NMR (300 MHz, DMSO) δ 9.24 (s, 1H), 7.64 (s, 1H), 7.46 (t, J=1.8 Hz, 1H), 6.33 (s, 1H), 1.44 (s, 9H). MS (ES) 184.20 (M+1).

### Step 2:

To a solution of tert-butyl furan-3-ylcarbamate (7G) (21 g, 114.65 mmol) in THF (800 mL) was added N,N,N′,N′-tetramethylene ethylenediamine (21.5 mL, 142.45 mmol). The resulting orange solution was cooled to −30° C. before treating dropwise with n-BuLi (1.6 M in hexanes, 157 mL, 250 mmol) and allowed to warm to 0° C. for 1 h after the addition of n-BuLi. The solution was again cooled to −30° C. and treated with dimethyl carbonate (28.75 mL, 341 mmol), allowed to warm to 0° C. over 45 min. The reaction was quenched with 2M HCl (400 mL) and extracted with ethyl acetate (800, 600, 400 mL). The combined organic layers were dried over MgSO₄, concentrated to dryness, and purified by flash column chromatography (silica gel, eluting with hexanes/ethyl acetate 0 to 100%) to give methyl 3-(tert-butoxycarbonylamino)furan-2-carboxylate (7H) (13.5 g, 49%) as a light brown oil. ¹H NMR (300 MHz, DMSO) δ 8.32 (s, 1H), 7.85 (s, 1H), 7.10 (s, 1H), 3.83 (s, 3H), 1.50 (s, 9H); MS (ES+) 264.1 (M+Na).

### Step 3:

To a solution of methyl 3-(tert-butoxycarbonylamino)furan-2-carboxylate (7H) (13.5 g, 55.96 mmol) in dichloromethane (100 mL) was added TFA (50 mL) and the resulting solution was stirred at room temperature for 5 h. The crude reaction mixture was concentrated under vacuum to dryness. The residue obtained was dissolved in dichloromethane (200 mL) and washed with sat. NaHCO₃ (3×100 mL). The organic layer was dried over MgSO₄, and concentrated in vacuum to dryness. The residue obtained was purified by flash column chromatography (silica gel, eluting with MeOH/CHCl₃ 0 to 20%) to furnish methyl 3-aminofuran-2-carboxylate (7E) (7.89 g, 100%) as a light yellow oil; ¹H NMR (300 MHz, CDCl₃) δ 7.26 (d, J=1.9 Hz, 1H), 6.13 (d, J=2.0 Hz, 1H), 4.51 (s, 2H), 3.88 (s, 3H). MS (ES+): 164.2 (M+Na).

### Step 4:

To a solution of methyl 3-aminofuran-2-carboxylate (7E) (0.35 g, 2.5 mmol) in dichloromethane (10 mL) was added at 0° C. sulfurisocyanatidic chloride (0.26 mL, 3.0 mmol) and stirred at 0° C. for 45 minutes. The reaction mixture was concentrated in vacuum to dryness and to the residue obtained was added acetic acid (0.5 mL), water (1 mL) and stirred at room temperature for 1 h. The reaction mixture was neutralized to pH 8 using saturated aqueous NaHCO₃ and the solid obtained was collected by filtration, dried in vacuum to furnish methyl 3-ureidofuran-2-carboxylate (7I) (0.29 g, 63%) as a white solid; mp 208.1° C.; ¹H NMR (300 MHz, DMSO) δ 8.46 (s, 1H), 7.73 (d, J=1.8 Hz, 1H), 7.27 (d, J=1.8 Hz, 1H), 6.70 (s, 2H), 3.82 (s, 3H).

### Step 5:

To a solution of methyl 3-ureidofuran-2-carboxylate (7I) (7.37 g, 40 mmol) was added aqueous NaOH (1.5 N, 210 mL, 315 mmol) and heated at reflux for 1.5 h. The pH of the reaction mixture was adjusted between 4-5 using aqueous HCl (6 N) and concentrated to (100 mL) volume. The solid obtained was collected by filtration dried in vacuum to furnish furo[3,2-d]pyrimidine-2,4-diol (7J) (4.56 g, 75%) as a brown solid; mp 232.9° C.; ¹H NMR (300 MHz, DMSO) δ 11.23 (s, 1H), 11.09 (s, 1H), 8.04 (d, J=2.0 Hz, 1H), 6.54 (d, J=2.0 Hz, 1H).

### Step 6:

To furo[3,2-d]pyrimidine-2,4-diol (7J) (1.52 g, 10 mmol) was added dimethyl aniline (1 mL, 8 mmol), phosphorous oxychloride (0.9 mL, 9.65 mmol) and heated at 130° C. for 3 h. The reaction mixture was cooled to room temperature and quenched carefully with ice. The solid obtained was collected by filtration washed with water and dried in vacuum to furnish 2,4-dichlorofuro[3,2-d]pyrimidine (7K) (1.02 g, 54%) as a light brown solid; mp 107.3° C.; ¹H NMR (300 MHz, CDCl₃) δ 8.08 (d, J=2.2, 1H), 7.02 (d, J=2.2, 1H).

### Example 2

### N-cyclobutyl-5-(2-(2-(pyridin-2-yl)pyrrolidin-1-yl)furo[3,2-d]pyrimidin-4-ylamino)-1H-pyrazole-3-carboxamide (13E)

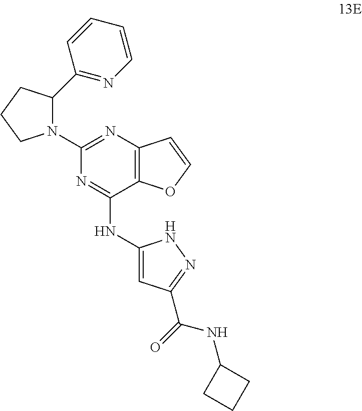

To a solution of 5-(2-chlorofuro[3,2-d]pyrimidin-4-ylamino)-N-cyclobutyl-1H-pyrazole-3-carboxamide (13D) (0.124 g, 0.37 mmol) in DMF (0.5 mL) was added 2-(pyrrolidin-2-yl)pyridine hydrochloride (12F) (0.086 g, 0.47 mmol), DIPEA (0.18 mL, 1.88 mmol) and heated in a microwave for 2 h at 180° C. The reaction mixture was concentrated in vacuo and purified by flash column chromatography [silica gel, eluting with eluting with 0-100% (9:1) ethyl acetate/methanol in hexanes] to furnish, N-cyclobutyl-5-(2-(2-(pyridin-2-yl)pyrrolidin-1-yl)furo[3,2-d]pyrimidin-4-ylamino)-1H-pyrazole-3-carboxamide (13E) (0.05 g, 30%) as an off-white solid; mp 250.1° C.; ¹H NMR (300 MHz, DMSO) δ 13.05-12.87 (bs, 0.67H), 12.87-12.70 (bs, 0.33H), 10.58-10.39 (bs, 0.33H), 10.12-9.92 (bs, 0.67H), 8.64-8.39 (m, 1.67H), 8.35-8.17 (m, 0.33H), 8.15-7.97 (m, 1H), 7.73-7.57 (m, 1H), 7.20 (m, 2H), 6.87-6.65 (m, 1H), 5.48-5.15 (m, 1H), 4.54-4.33 (m, 1H), 4.00-3.83 (m, 1H), 3.80-3.64 (m, 1H), 2.45-2.07 (m, 6H), 2.04-1.85 (m 3H), 1.81-1.56 (m, 2H). MS (ES+) 445.10 (M+1), ES(−) 479.1 (M+Cl).

### Preparation of intermediate compound 5-(2-chlorofuro[3,2-d]pyrimidin-4-ylamino)-N-cyclobutyl-1H-pyrazole-3-carboxamide (13D)

### Step 1:

A solution of 2M oxalyl chloride (in dichloromethane, 7 mL, 14 mmol) was added to a suspension of 5-nitro-3-pyrazocarboxylic acid (12A) (1.10 g, 7 mmol) in dichloromethane (25 mL), THF (0.7 mL) at 0° C. One drop of DMF was added to the reaction mixture and stirred at room temperature for 3 h. The reaction mixture was evaporated in vacuum to dryness and the residue obtained was dissolved in dichloromethane (35 mL). To the solution was added a mixture of cyclobutylamine (0.72 mL, 8.5 mmol), pyridine (1.13 mL), and dichloromethane (2 mL) over a period of 10 min. After stirring at room temperature overnight, the reaction mixture was concentrated in vacuum to dryness and the residue obtained was purified by flash column chromatography (silica gel, eluting with hexanes/ethyl acetate 0 to 100%) to furnish N-cyclobutyl-5-nitro-1H-pyrazole-3-carboxamide (13B) (0.927 g, 67.5%) as an off-white solid; mp 240.1° C.; ¹H NMR (300 MHz, DMSO) δ 14.77 (s, 1H), 8.92 (d, J=7.5 Hz, 1H), 7.65 (s, 1H), 4.55-4.24 (m, 1H), 2.33-2.15 (m, 2H), 2.13-1.95 (m, 2H), 1.79-1.60 (m, 2H). MS (ES−): 209.0 M−1.

### Step 2:

To a solution of N-cyclobutyl-5-nitro-1H-pyrazole-3-carboxamide (13B) (0.77 g, 3.7 mmol) in ethanol (20 mL) was added platinum oxide (125 mg) and hydrogenated at 60 psi for 3 h. The catalyst was removed by filtration through a pad of celite and the filtrate concentrated in vacuo to give 5-amino-N-cyclobutyl-1H-pyrazole-3-carboxamide (13C) (0.578 g, 87%) as a dark pink solid; mp 147.0° C.; ¹H NMR (300 MHz, MeOD) δ 5.87 (s, 1H), 4.44 (m, 1H), 2.32 (m, 2H), 2.15-1.97 (m, 2H), 1.75 (m, 2H); ¹H NMR (300 MHz, DMSO) δ 11.87 (s, 1H), 8.47-7.81 (m, 1H), 6.16-5.42 (m, 1H), 5.07 (bs, 2H), 4.34 (dd, J=8.3, 16.6 Hz, 1H), 2.06 (m, 4H), 1.61 (m, 2H). MS (ES−) 215.0 (M+Cl).

### Step 3:

To a solution of 2,4-dichlorofuro[3,2-d]pyrimidine (7K) (0.19 g, 1 mmol) in isopropanol (10 mL) was added diisopropylamine (0.231 mL, 1.33 mmol), 5-amino-N-cyclobutyl-1H-pyrazole-3-carboxamide (13C) (0.21 g, 1.1 mmol) and heated at reflux for 72 h. The reaction mixture was concentrated in vacuo to dryness and the residue obtained was purified by flash column chromatography [silica gel 24 g, eluting with 0-100% CMA-80 (chloroform:Methanol:conc. ammonia 80:18:2) in chloroform] to furnish 5-(2-chlorofuro[3,2-d]pyrimidin-4-ylamino)-N-cyclobutyl-1H-pyrazole-3-carboxamide (13D) (0.2 g, 57%) as an off-white solid; mp 277.1° C.; ¹H NMR (300 MHz, DMSO) δ 13.24 (s, 1H), 10.78 (s, 1H), 8.75 (d, J=7.6 Hz, 1H), 8.38 (d, J=2.0 Hz, 1H), 7.14 (s, 1H), 7.04 (d, J=2.0 Hz, 1H), 4.41 (dd, J=8.2, 16.3 Hz, 1H), 2.21 (m, 2H), 2.15-2.02 (m, 2H), 1.68 (m, 2H). MS (ES−) 330.90 (M−1).

### Example 3

### N-cyclobutyl-3-(2-(2-(pyridin-2-yl)pyrrolidin-1-yl)furo[3,2-d]pyrimidin-4-ylamino)-1H-pyrazole-5-carboxamide (14F)

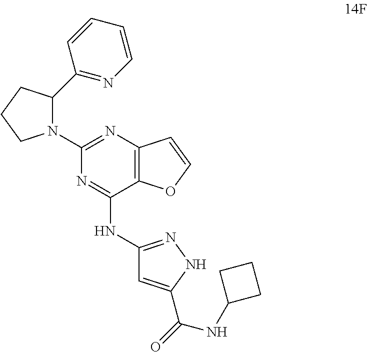

To a solution of 3-(2-(2-(pyridin-2-yl)pyrrolidin-1-yl)furo[3,2-d]pyrimidin-4-ylamino)-1H-pyrazole-5-carboxylic acid (14E) (0.065 g, 0.16 mmol) in DMF (1 mL) was added HATU (0.076 g, 0.2 mmol), DIPEA (0.069 mL, 0.4 mmol) and cyclobutylamine (0.018 mL, 0.21 mmol). The reaction mixture was heated at 70° C. for 2 h in a microwave and concentrated in vacuo to dryness. The residue obtained was purified twice by flash column chromatography [first column silica gel 12 g, eluting with 0-100% CMA-80 in chloroform followed by 0-100% CMA-50 in CMA-80, second column silica gel 12 g, eluting with 0-100% CMA-80 in chloroform] to furnish N-cyclobutyl-3-(2-(2-(pyridin-2-yl)pyrrolidin-1-yl)furo[3,2-d]pyrimidin-4-ylamino)-1H-pyrazole-5-carboxamide (14F) (17 mg, 23%) as a off-white; mp 274.3° C.; ¹H NMR (300 MHz, DMSO) δ 13.04-12.86 (bs, 0.64H), 12.85-12.69 (bs, 0.36H), 10.57-10.37 (m, 0.36H), 10.09-9.93 (bs, 0.64H), 8.63-8.39 (m, 1.67H), 8.33-8.18 (m, 0.33H), 8.16-7.99 (m, 1H), 7.74-7.57 (m, 1H), 7.20 (m, 2H), 6.86-6.62 (m, 1H), 5.45-5.17 (m, 1H), 4.55-4.35 (m, 1H), 4.01-3.84 (m, 1H), 3.80-3.66 (m, 1H), 2.44-2.06 (m, 6H), 2.05-1.86 (m, 3H), 1.80-1.58 (m, 2H); MS (ES+) 445.16 (M+1), 911.34 (2M+Na), ES(−) 443.1 (M−1).

### Preparation of intermediate compound 3-(2-(2-(pyridin-2-yl)pyrrolidin-1-yl)furo[3,2-d]pyrimidin-4-ylamino)-1H-pyrazole-5-carboxylic acid (14E)

### Step 1:

To a solution of methyl 3-nitro-1H-pyrazole-5-carboxylate (14A) (5 g, 29.22 mmol) in methanol (75 mL) was added Pd/C (10% on C, 0.6 g). The resulting mixture was hydrogenated at 50 psi for 24 h. After filtering the catalyst through a pad of Celite, the filtrate was concentrated in vacuum to dryness and the residue was purified by flash column chromatography (silica gel, eluting with MeOH/CHCl₃ 0 to 20%) to furnish methyl 3-amino-1H-pyrazole-5-carboxylate (14B) (4.4 g, 100%) as an off-white solid; mp 134.1° C.; ¹H NMR (300 MHz, DMSO) δ 12.99-11.69 (bs, 1H), 5.77 (s, 1H), 5.03 (bs, 2H), 3.74 (s, 3H). MS (ES+) 142.2 (M+1).

### Step 2:

To a solution of 2,4-dichlorofuro[3,2-d]pyrimidine (7K) (0.28 g, 1.5 mmol) in isopropanol (15 mL) was added diisopropylamine (0.653 mL, 3.75 mmol), methyl 3-amino-1H-pyrazole-5-carboxylate (14B) (0.25 g, 1.8 mmol) and heated at reflux for 48 h. The reaction mixture was concentrated in vacuo to dryness and the residue obtained was triturated with water. The solid obtained was collected by filtration to furnish on drying in vacuum methyl 3-(2-chlorofuro[3,2-d]pyrimidin-4-ylamino)-1H-pyrazole-5-carboxylate (14C) (0.23 g, 52%) as an off-white solid; mp 274.9° C.; ¹H NMR (300 MHz, DMSO) δ 13.78 (s, 1H), 11.06 (s, 1H), 8.40 (d, J=2.0 Hz, 1H), 7.18 (s, 1H), 7.07 (d, J=2.1 Hz, 1H), 3.87 (s, 3H). MS ES(−) 292.2 (M−1).

### Step 3:

To a solution of methyl 3-(2-chlorofuro[3,2-d]pyrimidin-4-ylamino)-1H-pyrazole-5-carboxylate (14C) (0.357 g, 1.22 mmol) in ethanol (1.25 mL) was added aqueous 1 N NaOH (1.25 mL, 1.25 mmol) and heated at reflux for 24 h. The reaction mixture was concentrated in vacuum and the residue obtained was triturated with water. The solid was collected by filtration dried in vacuum to furnish sodium 3-(2-chlorofuro[3,2-d]pyrimidin-4-ylamino)-1H-pyrazole-5-carboxylate (14D) (0.3 g, 83%) as a beige solid; mp 274.3° C.; ¹H NMR (300 MHz, DMSO) δ 12.42 (s, 1H), 10.61 (s, 1H), 8.32 (s, 1H), 7.00 (s, 1H), 6.63 (s, 1H). MS (ES−) 277.7 (M-Na).

### Step 4:

To 2-(pyrrolidin-2-yl)pyridine Hydrochloride (12F) (0.101 g, 0.547 mmol) in xylene (0.4 mL) was added DIPEA (0.095 mL, 0.547 mmol) and stirred at room temperature for 15 mins. To the reaction mixture was added Sodium 3-(2-chlorofuro[3,2-d]pyrimidin-4-ylamino)-1H-pyrazole-5-carboxylate (14D) (0.083 g, 0.274 mmol) and heated in a microwave for 5 h at 170° C. The reaction mixture was quenched with acetic acid (0.25 mL, 4.25 mmol) and concentrated in vacuum. The residue obtained was purified by flash column chromatography [silica gel, eluting with 0-100% CMA-80 (chloroform:Methanol:conc. ammonia 80:18:2) in chloroform followed with 0-100% CMA-50 (chloroform:Methanol:conc. ammonia 50:40:10) in CMA-80] to furnish 3-(2-(2-(pyridin-2-yl)pyrrolidin-1-yl)furo[3,2-d]pyrimidin-4-ylamino)-1H-pyrazole-5-carboxylic acid (14E) (77 mg, 72% contaminated with ammonium acetate) which was used as such for next step. MS (ES+) 392.1 (M+1).

### Example 4

### (S)—N-cyclopropyl-3-(2-(2-(hydroxymethyl)pyrrolidin-1-yl)furo[3,2-d]pyrimidin-4-ylamino)-1H-pyrazole-5-carboxamide (15C)

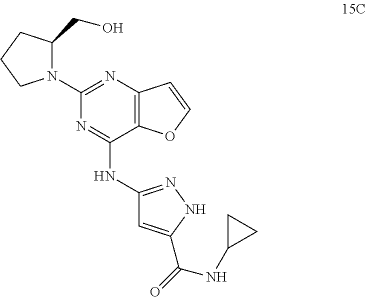

To a solution of (S)-3-(2-(2-(hydroxymethyl)pyrrolidin-1-yl)furo[3,2-d]pyrimidin-4-ylamino)-1H-pyrazole-5-carboxylic acid (15B) (0.11 g, 0.33 mmol) in DMF (2 mL) was added HATU (0.16 g, 0.42 mmol), DIPEA (0.072 mL, 0.42 mmol) and cyclopropylamine (0.116 mL, 1.65 mmol). The reaction mixture was stirred at room temperature overnight and concentrated in vacuo to dryness. The residue obtained was purified twice by flash column chromatography [first column silica gel 24 g, eluting with 0-100% CMA-80 in chloroform, second column silica gel 12 g, eluting with 0-100% CMA-80 in chloroform] to furnish (S)—N-Cyclopropyl-3-(2-(2-(hydroxymethyl)pyrrolidin-1-yl)furo[3,2-d]pyrimidin-4-ylamino)-1H-pyrazole-5-carboxamide (15C) (18 mg, 15%) as a as a beige solid; mp 96.8° C.; ¹H NMR (300 MHz, DMSO) δ 13.07 (s, 0.7H), 12.91-12.65 (bs, 0.3H), 10.60-10.38 (bs, 0.3H), 10.34-10.04 (bs, 0.7H), 8.05 (m, 2H), 7.25 (s, 0.7H), 6.75 (m, 1H), 6.57-6.36 (m, 0.3H), 5.63-5.24 (m, 0.7H), 5.16-4.97 (m, 0.3H), 4.28-4.00 (m, 1H), 3.89-3.69 (m, 1H), 3.56-3.45 (m, 1H), 3.30-3.21 (m, 1H), 2.81 (m, 1H), 1.92 (m, 4H), 1.23 (m, 1H), 0.63 (m, 4H). MS (ES+) 384.1 (M+1), ES (−) 382.3 (M−1).

### Preparation of intermediate compound (S)-3-(2-(2-(hydroxymethyl)-pyrrolidin-1-yl)furo[3,2-d]pyrimidin-4-ylamino)-1H-pyrazole-5-carboxylic acid (15B)

To (S)-pyrrolidin-2-ylmethanol (15A) (0.17 mL, 1.75 mmol) in xylene (0.8 mL) was added Sodium 3-(2-chlorofuro[3,2-d]pyrimidin-4-ylamino)-1H-pyrazole-5-carboxylate (14D) (0.212 g, 0.7 mmol) and heated in a microwave for 4 h at 200° C. The reaction mixture was quenched with acetic acid (0.4 mL) and concentrated in vacuum. The residue obtained was purified by flash column chromatography [silica gel 12 g, eluting with 0-100% CMA-80 (chloroform:Methanol:conc. ammonia 80:18:2) in chloroform followed with 0-100% CMA-50 (chloroform:Methanol:conc. ammonia 50:40:10) in CMA-80] to furnish (S)-3-(2-(2-(hydroxymethyl)pyrrolidin-1-yl)furo[3,2-d]pyrimidin-4-ylamino)-1H-pyrazole-5-carboxylic acid (15B) (11 mg, 45%). MS (ES+) 345.1 (M+1), (ES−) 343.02 (M−1).

### Example 5

### N-(3-methoxyphenyl)-3-(2-(2-(pyridin-2-yl)pyrrolidin-1-yl)furo[3,2-c]pyrimidin-4-ylamino)-1H-pyrazole-5-carboxamide (16D)

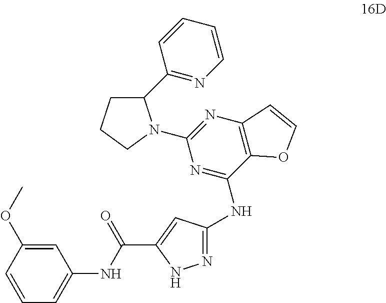

To a solution of 3-(2-chlorofuro[3,2-d]pyrimidin-4-ylamino)-N-(3-methoxyphenyl)-1H-pyrazole-5-carboxamide (16C) (0.043 g, 0.11 mmol) in NMP (0.5 mL) was added 2-(pyrrolidin-2-yl)pyridine Hydrochloride (12F) (0.050 g, 0.22 mmol), DIPEA (0.078 mL, 0.444 mmol) and heated in a microwave for 3 h at 200° C. The reaction mixture was cooled to room temperature diluted with water (5 mL) and extracted with ethyl acetate (3×5 mL). The organic layers were combined, washed with water (5 mL), brine (5 mL), dried, and concentrated in vacuo. The residue obtained was purified twice by flash column chromatography [silica gel 12 g and 4 g, eluting with 0-100% (9:1) ethyl acetate/methanol in hexanes] to furnish N-(3-methoxyphenyl)-3-(2-(2-(pyridin-2-yl)pyrrolidin-1-yl)furo[3,2-d]pyrimidin-4-ylamino)-1H-pyrazole-5-carboxamide (16D) (0.02 g, 36%) as an off-white solid; NMR (300 MHz, DMSO) δ 13.31-13.14 (m, 0.6H), 13.06-12.88 (m, 0.4H), 10.66-10.43 (m, 0.4H), 10.26-10.08 (m, 1.2H), 10.04-9.88 (m, 0.4H), 8.61-8.45 (m, 0.6H), 8.39-8.20 (m, 0.4H), 8.19-8.00 (m, 1H), 7.72-7.42 (m, 3H), 7.18 (s, 3H), 6.86-6.63 (m, 2H), 5.59-5.36 (m, 0.4H), 5.35-5.20 (m, 0.6H), 4.03-3.85 (m, 1H), 3.76 (s, 4H), 3.31 (s, 1H), 2.43-2.31 (m, 1H), 1.98 (s, 3H). MS (ES(−) 495.0 (M−1).

### Preparation of intermediate compound 3-(2-chlorofuro[3,2-d]pyrimidin-4-ylamino)-N-(3-methoxyphenyl)-1H-pyrazole-5-carboxamide (16C)

### Step 1:

To a solution of methyl 3-(2-chlorofuro[3,2-d]pyrimidin-4-ylamino)-1H-pyrazole-5-carboxylate (14C) (0.55 g, 1.87 mmol) in ethanol (2 mL) was added aqueous 1 N NaOH (2 mL, 2 mmol) and heated at reflux for 48 h. The reaction mixture was quenched with acetic acid (0.6 mL) and concentrated in vacuum to dryness. The residue obtained was triturated with water (5 mL) and IPA (5 mL). The solid obtained was collected by filtration dried in vacuum to furnish 3-(2-chlorofuro[3,2-d]pyrimidin-4-ylamino)-1H-pyrazole-5-carboxylic acid (16A) (0.34 g, 60%) as a off white solid; ¹HNMR (300 MHz, DMSO) δ 13.87-13.22 (m, 1H), 10.99 (s, 1H), 8.39 (d, J=2.1, 1H), 7.08 (s, 1H), 7.06 (d, J=2.2, 1H); MS (ES−) 278.3 (M−1).

### Step 2:

To a solution of 3-(2-chlorofuro[3,2-d]pyrimidin-4-ylamino)-1H-pyrazole-5-carboxylic acid ((16A) (0.38 g, 1.36 mmol) in DMF (5 mL) was added HATU (0.91 g, 2.4 mmol), DIPEA (0.627 mL, 3.6 mmol) and 3-methoxyaniline (16B) (0.27 mL, 2.4 mmol). The reaction mixture was stirred at room temperature overnight and diluted with water (15 mL). The reaction mixture was extracted with ethyl acetate (3×10 mL). The organic layers were combined, washed with water (10 mL), brine (10 mL), dried, and concentrated in vacuo. The residue obtained was purified by flash column chromatography [silica gel 24 g eluting with 0-100% (9:1) ethyl acetate/methanol in hexanes] to furnish 3-(2-chlorofuro[3,2-d]pyrimidin-4-ylamino)-N-(3-methoxyphenyl)-1H-pyrazole-5-carboxamide (16C) (0.043 g, 18%) as an off-white solid; MS (ES−) 383.2 (M−1).

### Example 6

### 3-(2-(2-(pyridin-2-yl)pyrrolidin-1-yl)furo[3,2-d]pyrimidin-4-ylamino)-N-(pyridin-4-yl)-1H-pyrazole-5-carboxamide (17C)

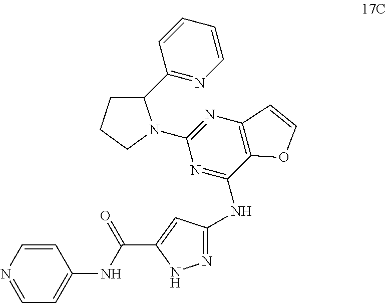

To a solution of 3-(2-chlorofuro[3,2-d]pyrimidin-4-ylamino)-N-(pyridin-4-yl)-1H-pyrazole-5-carboxamide (17B) (0.102 g, 0.29 mmol) in NMP (0.5 mL) was added 2-(pyrrolidin-2-yl)pyridine Hydrochloride (12F) (0.127 g, 0.574 mmol), DIPEA (0.2 mL, 1.15 mmol) and heated in a microwave for 3 h at 200° C. The reaction mixture was diluted with water (5 mL) and extracted with ethyl acetate (3×5 mL). The organic layers were combined, washed with water (5 mL), brine (5 mL), dried, and concentrated in vacuo. The residue obtained was purified twice by flash column chromatography [silica gel 12 g and 4 g, eluting with 0-100% (9:1) ethyl acetate/methanol in hexanes] to furnish 3-(2-(2-(pyridin-2-yl)pyrrolidin-1-yl)furo[3,2-d]pyrimidin-4-ylamino)-N-(pyridin-4-yl)-1H-pyrazole-5-carboxamide (17C) (0.01 g, 8%) as an off-white solid; ¹H NMR (300 MHz, DMSO) δ 13.47-13.24 (m, 0.5H), 13.22-13.00 (m, 0.5H), 10.67-10.37 (m, 1.5H), 10.29-10.12 (m, 0.5H), 8.49 (s, 3H), 8.23-8.05 (m, 1H), 7.88 (s, 2H), 7.74-7.59 (m, 1H), 7.19 (s, 2H), 6.88-6.66 (m, 1H), 5.56-5.20 (m, 1H), 3.99-3.85 (m, 1H), 3.82-3.69 (m, 1H), 3.29-3.22 (m, 1H), 2.39-2.33 (m, 1H), 1.99 (s, 3H). MS ES (+) 468.03, ES (−) 466.1 (M−1).

### Preparation of intermediate compound 3-(2-chlorofuro[3,2-d]pyrimidin-4-ylamino)-N-(pyridin-4-yl)-1H-pyrazole-5-carboxamide (17B)

To a solution of 3-(2-chlorofuro[3,2-d]pyrimidin-4-ylamino)-1H-pyrazole-5-carboxylic acid (16A) (0.17 g, 0.6 mmol) in DMF (2 mL) was added HATU (0.464 g, 1.22 mmol), DIPEA (0.212 mL, 1.22 mmol) and pyridin-4-amine (17A) (0.115 g, 1.22 mmol). The reaction mixture was stirred at room temperature overnight and diluted with water (10 mL). The reaction mixture was extracted with ethyl acetate (3×10 mL). The organic layers were combined, washed with water (10 mL), brine (10 mL), dried, and concentrated in vacuo. The residue obtained was purified by flash column chromatography [silica gel 12 g eluting with 0-100% (9:1) ethyl acetate/methanol in hexanes] to furnish 3-(2-chlorofuro[3,2-d]pyrimidin-4-ylamino)-N-(pyridin-4-yl)-1H-pyrazole-5-carboxamide (17B) (0.102 g, 47%) as an off-white solid; MS (ES+) 356.0 (M+1), (ES−) 353.8 (M−1).

### Example 7

### 3-Amino-1-(2-chlorofuro[3,2-d]pyrimidin-4-yl)-N-cyclopropyl-1H-pyrazole-5-carboxamide (18A)

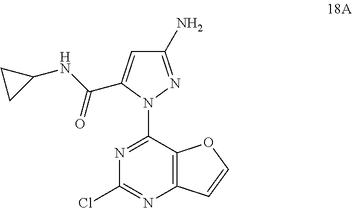

To a solution of 2,4-dichlorofuro[3,2-d]pyrimidine (7K) (0.19 g, 1 mmol) in isopropanol (10 mL) was added solid NaHCO₃ (1.68 g, 2 mmol), 5-amino-N-cyclopropyl-1H-pyrazole-3-carboxamide (12C) (0.2 g, 1.2 mmol) and heated at reflux for 48 h. The reaction mixture was concentrated in vacuo to dryness and the residue obtained was purified by flash column chromatography [silica gel, 24 g, eluting with 0-100% (9:1) ethyl acetate/methanol in hexanes] to furnish. 5-(2-Chlorofuro[3,2-d]pyrimidin-4-ylamino)-N-cyclopropyl-1H-pyrazole-3-carboxamide (12D) and 3-Amino-1-(2-chlorofuro[3,2-d]pyrimidin-4-yl)-N-cyclopropyl-1H-pyrazole-5-carboxamide (18A) (0.02 g, 6%) as a yellow solid; mp 218.8° C.; ¹H NMR (300 MHz, DMSO) δ 8.73 (d, J=2.2 Hz, 1H), 8.07 (d, J=4.2 Hz, 1H), 7.31 (d, J=2.2 Hz, 1H), 6.92 (s, 2H), 5.82 (s, 1H), 2.81 (dt, J=5.6, 11.3 Hz, 1H), 0.75-0.66 (m, 2H), 0.64-0.56 (m, 2H); ¹H NMR (300 MHz, MeOD) δ 8.45 (d, J=2.2 Hz, 1H), 7.10 (d, J=2.2 Hz, 1H), 5.91 (s, 1H), 2.89-2.76 (m, 1H), 0.84 (m, 2H), 0.73-0.63 (m, 2H).

### Example 8

### N-cyclobutyl-5-(2-(dimethylamino)furo[3,2-d]pyrimidin-4-ylamino)-1H-pyrazole-3-carboxamide (19A)

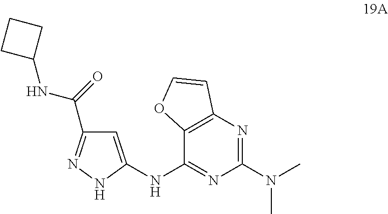

To a solution of 5-(2-chlorofuro[3,2-d]pyrimidin-4-ylamino)-N-cyclobutyl-1H-pyrazole-3-carboxamide (13D) (0.124 g, 0.37 mmol) in DMF (0.5 mL) was added 2-(pyrrolidin-2-yl)pyridine hydrochloride (12F) (0.086 g, 0.47 mmol), DIPEA (0.18 mL, 1.88 mmol) and heated in a microwave for 2 h at 180° C. The reaction mixture was concentrated in vacuo and purified by flash column chromatography [silica gel eluting with 0-100% (9:1) ethyl acetate/methanol in hexanes] to furnish N-cyclobutyl-5-(2-(dimethylamino)furo[3,2-d]pyrimidin-4-ylamino)-1H-pyrazole-3-carboxamide (19A) (0.052 g, 41%) as an off-white solid; mp 270.8° C.; ¹H NMR (300 MHz, DMSO) δ 13.12-12.88 (bs, 0.7H), 12.85-12.56 (bs, 0.3H), 10.43-10.14 (bs, 0.3H), 10.08-9.78 (s, 0.7H), 8.69-8.44 (m, 0.7H), 8.28-8.17 (m, 0.3H), 8.15-7.96 (m, 1H), 7.18 (s, 0.7H), 6.73 (m, 1H), 6.57-6.40 (m, 0.3H), 4.37 (m, 1H), 3.11 (s, 6H), 2.20 (m, 4H), 1.67 (m, 2H). MS (ES−) 339.99 (M−1). N-Cyclobutyl-5-(2-(2-(pyridin-2-yl)pyrrolidin-1-yl)furo[3,2-d]pyrimidin-4-ylamino)-1H-pyrazole-3-carboxamide (13E) (0.05 g, 30%) as an off-white solid; mp 250.1° C.; ¹H NMR (300 MHz, DMSO) δ 13.05-12.87 (bs, 0.67H), 12.87-12.70 (bs, 0.33H), 10.58-10.39 (bs, 0.33H), 10.12-9.92 (bs, 0.67H), 8.64-8.39 (m, 1.67H), 8.35-8.17 (m, 0.33H), 8.15-7.97 (m, 1H), 7.73-7.57 (m, 1H), 7.20 (m, 2H), 6.87-6.65 (m, 1H), 5.48-5.15 (m, 1H), 4.54-4.33 (m, 1H), 4.00-3.83 (m, 1H), 3.80-3.64 (m, 1H), 2.45-2.07 (m, 6H), 2.04-1.85 (m 3H), 1.81-1.56 (m, 2H). MS (ES+) 445.10 (M+1), ES(−) 479.1 (M+Cl).

### Example 9

The following illustrate representative pharmaceutical dosage forms, containing a compound of formula I (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 01N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

TABLE I
Activity for Representative Compounds of
the Invention for JAK Family of Enzymes

| Compound | Activity |
|---|---|
| 12E | IC₅₀ < 5 uM |
| 19A | IC₅₀ > 10 uM |
| 13E | IC₅₀ < 5 uM |
| 14F | IC₅₀ < 5 uM |
| 15C | IC₅₀ < 5 uM |
| 16D | IC₅₀ < 5 uM |
| 17C | IC₅₀ < 5 uM |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 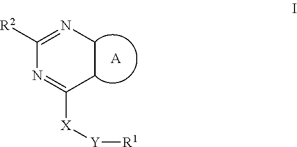
wherein:
A is furan optionally substituted with one or more R³ groups;
X is NH, O, S or absent;
Y is heteroaryl or aryl, wherein heteroaryl is linked to X by a carbon atom when X is NH, O or S and wherein any heteroaryl or aryl of Y may be optionally substituted with one or more Rₐ groups;
R¹ is —C(O)NR_{g}Rₕ, —C(S)NR_{g}Rₕ, or —C(═NRᵢ)NR_{g}Rₕ;
R² is heteroaryl, —NR⁶R⁷, —OR⁸, SR⁸ or CHR⁹R¹⁰, wherein any heteroaryl of R² may be optionally substituted with one or more R¹¹ groups;
each R³ is independently halo, (C₁-C₆)alkyl, (C₂-C₆)alkenyl, (C₂-C₆)alkynyl, (C₃-C₆)cycloalkyl, —ORₐ₂, —OC(O)R_{b2}, —OC(O)NR_{c2}R_{d2}, —SRₐ₂, —S(O)₂OH, —S(O)R_{b2}, —S(O)₂R_{b2}, —S(O)₂NR_{c2}R_{d2}, —NR_{c2}R_{d2}, —NRₑ₂C(O)R_{b2}, —NRₑ₂C(O)NR_{c2}R_{d2}, NRₑ₂S(O)₂R_{b2}, —NRₑ₂S(O)₂NR_{c2}R_{d2}, NO₂, —C(O)Rₐ₂, —C(O)ORₐ₂ or —C(O)NR_{c2}R_{d2};
R⁶ is selected from (C₁-C₆)alkyl, (C₂-C₆)alkenyl, (C₂-C₆)alkynyl, (C₃-C₆)cycloalkyl, heteroaryl, heterocycle and aryl, and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino or thiomorpholino of R⁶ and R⁷ may be optionally substituted with one or more R^{H} groups;
each R⁸ is independently selected from (C₁-C₆)alkyl, (C₂-C₆)alkenyl, (C₂-C₆)alkynyl, (C₃-C₆)cycloalkyl, heteroaryl and aryl, wherein any alkyl, alkenyl, alkynyl, cycloalkyl, heteroaryl or aryl of R⁸ may be optionally substituted with one or more R¹¹ groups;
R⁹ is selected from (C₁-C₆)alkyl, (C₂-C₆)alkenyl, (C₂-C₆)alkynyl, (C₃-C₆)cycloalkyl, heteroaryl, heterocycle and aryl, and R¹⁰ is selected from H and (C₁-C₆)alkyl; or R⁹ and R¹⁰ together with the carbon to which they are attached form a (C₃-C₇)cycloalkyl, pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of R⁹ and R¹⁰ may be optionally substituted with one or more R¹¹ groups;
each R¹¹ is independently selected from (C₁-C₆)alkyl, (C₂-C₆)alkenyl, (C₂-C₆)alkynyl, (C₃-C₆)cycloalkyl, —ORₘ, —NRₜCORₙ, NRₒRₚ, heteroaryl and aryl, wherein alkyl, alkenyl, alkynyl, cycloalkyl, heteroaryl or aryl may be optionally substituted with one or more groups selected from halo, R_{q}, OH, CN, —OR_{q}, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, CHO, —C(O)R_{q}, CO₂H, —C(O)OR_{q} and —C(O)NRᵣRₛ;
each Rₐ is independently selected from (C₁-C₆)alkyl, (C₂-C₆)alkenyl, (C₂-C₆)alkynyl, (C₃-C₆)cycloalkyl, halo, CN, —OR_{f}, —OC(O)R_{b}, —OC(O)NR_{c}R_{d}, —SR_{f}, —S(O)R_{b}, —S(O)₂OH, —S(O)₂R_{b}, —S(O)₂NR_{c}R_{d}, —NR_{c}R_{d}, —NRₑCOR_{b}, —NRₑCO₂R_{b}, —NRₑCONR_{c}R_{d}, —NRₑS(O)₂R_{b}, —NRₑS(O)₂NR_{c}R_{d}, NO₂, —C(O)R_{f}, —C(O)OR_{f} and —C(O)NR_{c}R_{d};
each R_{b} is independently (C₁-C₆)alkyl, (C₂-C₆)alkenyl, (C₂-C₆)alkynyl, (C₃-C₆)cycloalkyl, heterocycle, heteroaryl or aryl;
R_{c} and R_{d} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c} and R_{d} together with the nitrogen to which they are attached form a pyrrolidino, piperidino, piperazino, azetidino, morpholino, or thiomorpholino;
each Rₑ is independently H, (C₁-C₆)alkyl, (C₂-C₆)alkenyl, (C₂-C₆) alkynyl or (C₃-C₆)cycloalkyl;
each R_{f} is independently H, (C₁-C₆)alkyl, (C₂-C₆)alkenyl, (C₂-C₆)alkynyl, (C₃-C₆)cycloalkyl, heterocycle, heteroaryl or aryl;
each R_{g} is independently selected from aryl, heterocycle and heteroaryl, wherein any aryl or heteroaryl of R_{g} may be optionally substituted with one or more Rₖ groups and wherein any heterocycle of R_{g} may be optionally substituted with one or more oxo (C═O) or Rₖ groups;
each Rₕ is independently selected from H, (C₁-C₈)alkyl, (C₂-C₈)alkenyl, (C₂-C₈)alkynyl, (C₃-C₈)cycloalkyl, heterocycle, heteroaryl and aryl, wherein any aryl or heteroaryl of Rₕ may be optionally substituted with one or more Rₖ groups and wherein any alkyl, alkenyl, alkynyl, cycloalkyl or heterocycle of Rₕ may be optionally substituted with one or more oxo (C═O) or Rₖ groups;
Rᵢ is independently H, (C₁-C₆)alkyl, (C₂-C₆)alkenyl, (C₂-C₆)alkynyl or (C₃-C₆)cycloalkyl;
each Rₖ is independently selected from halo, R_{y}, CN, OH, —OR_{y}, —OC(O)R_{y}, —OC(O)NRᵥR_{w}, SH, —SR_{y}, —S(O)R_{y}, —S(O)₂OH, —S(O)₂R_{y}, —S(O)₂NRᵥR_{w}, —NRᵥR_{w}, —NRₓCOR_{y}, —NRₓCO₂R_{y}, —NRₓCONRᵥR_{w}, —NRₓS(O)₂R_{y}, —NRₓS(O)₂NRᵥR_{w}, NO₂, —C(O)Rᵤ, —C(O)ORᵤ and —C(O)NRᵥR_{w};
each Rₘ is independently H, (C₁-C₆)alkyl, (C₂-C₆)alkenyl, (C₂-C₆)alkynyl, (C₃-C₆)cycloalkyl, heterocycle, heteroaryl or aryl;
each Rₙ is independently (C₁-C₆)alkyl, (C₂-C₆)alkenyl, (C₂-C₆)alkynyl, (C₃-C₆)cycloalkyl, heterocycle, heteroaryl or aryl;
Rₒ and Rₚ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rₒ and Rₚ together with the nitrogen to which they are attached form a pyrrolidino, piperidino, piperazino, azetidino, morpholino, or thiomorpholino;
each R_{q} is independently (C₁-C₆)alkyl, (C₂-C₆)alkenyl, (C₂-C₆)alkynyl, (C₃-C₆)cycloalkyl, heterocycle, heteroaryl or aryl;
Rᵣ and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino;
each Rₜ is independently H, (C₁-C₆)alkyl, (C₂-C₆)alkenyl, (C₂-C₆) alkynyl or (C₃-C₆)cycloalkyl;
each Rᵤ is independently H, (C₁-C₆)alkyl, (C₂-C₆)alkenyl, (C₂-C₆)alkynyl, (C₃-C₆)cycloalkyl, heterocycle, heteroaryl or aryl;
Rᵥ and R_{w} are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵥ and R_{w} together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of Rᵥ and R_{w} may be optionally substituted with one or more groups independently selected from OH, CH₂OH, NH₂ and CONH₂;
each Rₓ is independently H, (C₁-C₆)alkyl, (C₂-C₆)alkenyl, (C₂-C₆) alkynyl or (C₃-C₆)cycloalkyl;
each R_{y} is independently (C₁-C₆)alkyl, (C₂-C₆)alkenyl, (C₂-C₆)alkynyl, (C₃-C₆)cycloalkyl, heterocycle, heteroaryl or aryl, wherein any alkyl, alkenyl, alkynyl, cycloalkyl, heterocycle, heteroaryl or aryl of R_{y} may be optionally substituted with one or more groups selected from ORᵤ and NRᵥR_{w};
each Rₐ₂ is independently H, (C₁-C₆)alkyl, (C₂-C₆)alkenyl, (C₂-C₆)alkynyl, (C₃-C₆)cycloalkyl, heterocycle, heteroaryl or aryl;
each R_{b2} is independently (C₁-C₆)alkyl, (C₂-C₆)alkenyl, (C₂-C₆)alkynyl, (C₃-C₆)cycloalkyl, heterocycle, heteroaryl or aryl;
R_{c2} and R_{d2} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c2} and R_{d2} together with the nitrogen to which they are attached form a pyrrolidino, piperidino, piperazino, azetidino, morpholino, or thiomorpholino; and
each Rₑ₂ is independently H, (C₁-C₆)alkyl, (C₂-C₆)alkenyl, (C₂-C₆) alkynyl or (C₃-C₆)cycloalkyl;
or a salt thereof.

2. The compound of claim 1 which is a compound of formula IIb: 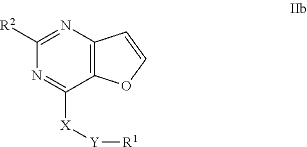
or a salt thereof.

3. The compound of claim 1 which is a compound of formula IIa: 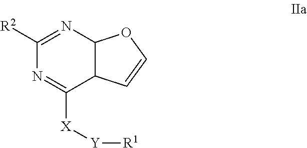
or a salt thereof.

4. The compound of claim 1 which is a compound of formula IIc: 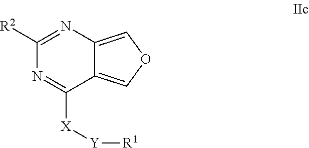
or a salt thereof.

5. The compound of claim 1 wherein R¹ is —C(O)NR_{g}Rₕ.

6-9. (canceled)

10. The compound of claim 1 wherein —X—Y—R¹ is:

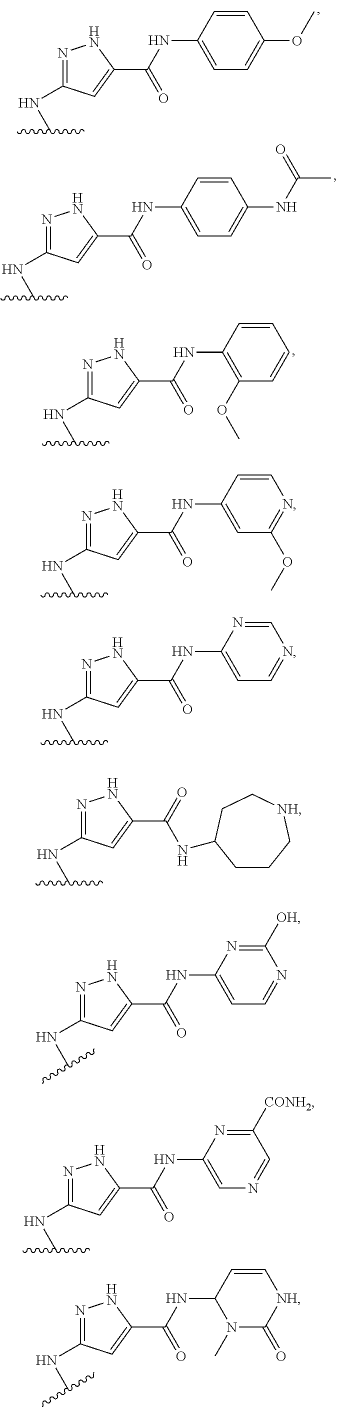

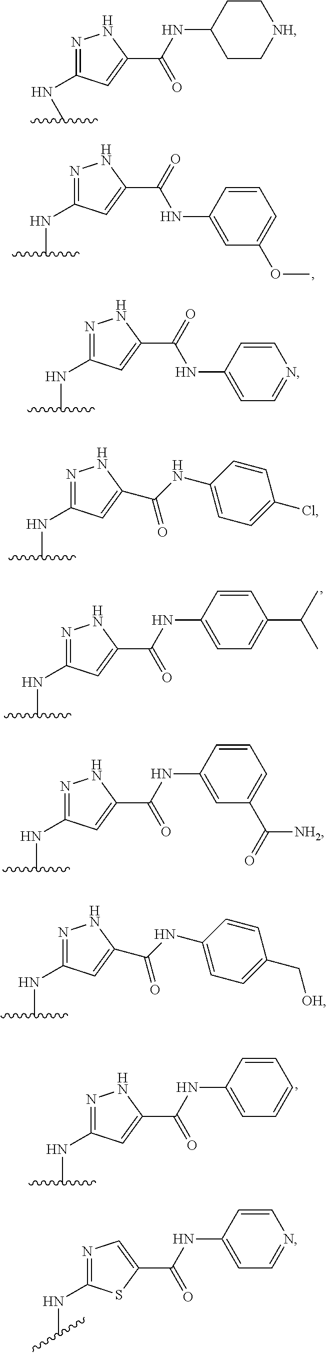

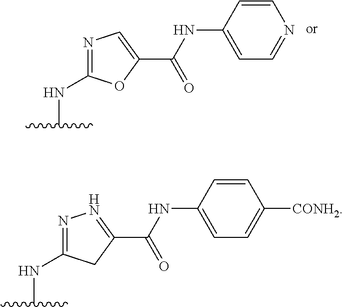

11. A compound of formula I: 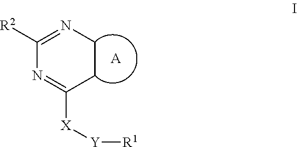
wherein:
A is furan optionally substituted with one or more R³ groups;
X is NH, O, S or absent;
Y is heteroaryl or aryl, wherein heteroaryl is linked to X by a carbon atom when X is NH, O or S and wherein any heteroaryl or aryl of Y may be optionally substituted with one or more Rₐ groups;
R¹ is —C(O)NR_{g1}Rₕ₁, —NRᵢC(O)NR_{g}Rₕ, —CHO, —C(O)Rⱼ, —CO₂H, —C(O)ORⱼ, —NRᵢS(O)₂NR_{g}Rₕ, —NRᵢC(O)Rⱼ, —NRᵢS(O)₂Rⱼ, —C(O)C(O)Rⱼ, —C(O)NRᵢS(O)₂Rⱼ, —C(O)NRᵢCHO, —C(O)NRᵢC(O)Rⱼ, —C≡CH, —C≡CRⱼ, —C(S)NR_{g1}Rₕ₁, —C(═NRᵢ)NR_{g1}Rₕ₁, (C₁-C₆)alkyl, (C₃-C₆)cycloalkyl, heterocycle, heteroaryl, aryl or is absent, wherein any alkyl, cycloalkyl, heterocycle, heteroaryl or aryl of R¹ may be optionally substituted with one or more R_{z} groups;
R² is heteroaryl, —NR⁶R⁷, —OR⁸, SR⁸ or CHR⁹R¹⁰, wherein any heteroaryl of R² may be optionally substituted with one or more R¹¹ groups;
each R³ is independently halo, (C₁-C₆)alkyl, (C₂-C₆)alkenyl, (C₂-C₆)alkynyl, (C₃-C₆)cycloalkyl, —ORₐ₂, —OC(O)R_{b2}, —OC(O)NR_{c2}R_{d2}, —SRₐ₂, —S(O)₂OH, —S(O)R_{b2}, —S(O)₂R_{b2}, —S(O)₂NR_{c2}R_{d2}, —NR_{c2}R_{d2}, —NRₑ₂C(O)R_{b2}, —NRₑ₂C(O)NR_{c2}R_{d2}, NRₑ₂S(O)₂R_{b2}, —NRₑ₂S(O)₂NR_{c2}R_{d2}, NO₂, —C(O)Rₐ₂, —C(O)ORₐ₂ or —C(O)NR_{c2}R_{d2};
R⁶ is selected from (C₁-C₆)alkyl, (C₂-C₆)alkenyl, (C₂-C₆)alkynyl, (C₃-C₆)cycloalkyl, heteroaryl, heterocycle and aryl, and R⁷ is selected from H and (C₁-C₆)alkyl; or R⁶ and R⁷ together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino or thiomorpholino of R⁶ and R⁷ may be optionally substituted with one or more R¹¹ groups;
each R⁸ is independently selected from (C₁-C₆)alkyl, (C₂-C₆)alkenyl, (C₂-C₆)alkynyl, (C₃-C₆)cycloalkyl, heteroaryl and aryl, wherein any alkyl, alkenyl, alkynyl, cycloalkyl, heteroaryl or aryl of R⁸ may be optionally substituted with one or more R¹¹ groups;
R⁹ is selected from (C₁-C₆)alkyl, (C₂-C₆)alkenyl, (C₂-C₆)alkynyl, (C₃-C₆)cycloalkyl, heteroaryl, heterocycle and aryl, and R¹⁰ is selected from H and (C₁-C₆)alkyl; or R⁹ and R¹⁰ together with the carbon to which they are attached form a (C₃-C₇)cycloalkyl, pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of R⁹ and R¹⁰ may be optionally substituted with one or more R¹¹ groups;
each R¹¹ is independently selected from (C₁-C₆)alkyl, (C₂-C₆)alkenyl, (C₂-C₆)alkynyl, (C₃-C₆)cycloalkyl, —ORₘ, —NRₜCORₙ, NRₒRₚ, heteroaryl and aryl, wherein alkyl, alkenyl, alkynyl, cycloalkyl, heteroaryl or aryl may be optionally substituted with one or more groups selected from halo, R_{q}, OH, CN, —OR_{q}, —OC(O)R_{q}, —OC(O)NRᵣRₛ, SH, —SR_{q}, —S(O)R_{q}, —S(O)₂OH, —S(O)₂R_{q}, —S(O)₂NRᵣRₛ, —NRᵣRₛ, —NRₜCOR_{q}, —NRₜCO₂R_{q}, —NRₜCONRᵣRₛ, —NRₜS(O)₂R_{q}, —NRₜS(O)₂NRᵣRₛ, NO₂, —CHO, —C(O)R_{q}, —C(O)OH, —C(O)OR_{q} and —C(O)NRᵣRₛ;
each Rₐ is independently selected from (C₁-C₆)alkyl, (C₂-C₆)alkenyl, (C₂-C₆)alkynyl, (C₃-C₆)cycloalkyl, halo, CN, —OR_{f}, —OC(O)R_{b}, —OC(O)NR_{c}R_{d}, —SR_{f}, —S(O)R_{b}, —S(O)₂OH, —S(O)₂R_{b}, —S(O)₂NR_{c}R_{d}, —NR_{c}R_{d}, —NRₑCOR_{b}, —NRₑCO₂R_{b}, —NRₑCONR_{c}R_{d}, —NRₑS(O)₂R_{b}, —NRₑS(O)₂NR_{c}R_{d}, NO₂, —C(O)R_{f}, —C(O)OR_{f} and —C(O)NR_{c}R_{d};
each R_{b} is independently (C₁-C₆)alkyl, (C₂-C₆)alkenyl, (C₂-C₆)alkynyl, (C₃-C₆)cycloalkyl, heterocycle, heteroaryl or aryl;
R_{c} and R_{d} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c} and R_{d} together with the nitrogen to which they are attached form a pyrrolidino, piperidino, piperazino, azetidino, morpholino, or thiomorpholino;
each Rₑ is independently H, (C₁-C₆)alkyl, (C₂-C₆)alkenyl, (C₂-C₆) alkynyl or (C₃-C₆)cycloalkyl;
each R_{f} is independently H, (C₁-C₆)alkyl, (C₂-C₆)alkenyl, (C₂-C₆)alkynyl, (C₃-C₆)cycloalkyl, heterocycle, heteroaryl or aryl;
R_{g1} is selected from H, (C₁-C₈)alkyl, (C₂-C₈)alkenyl, (C₂-C₈)alkynyl or (C₃-C₈)cycloalkyl, wherein any alkyl, alkenyl, alkynyl or cycloalkyl of R_{g1} may be optionally substituted with one or more oxo (C═O) or Rₖ groups, and Rₕ₁ is selected from H, (C₁-C₈)alkyl, (C₂-C₈)alkenyl, (C₂-C₈)alkynyl or (C₃-C₈)cycloalkyl, wherein any alkyl, alkenyl, alkynyl or cycloalkyl of Rₕ₁ may be optionally substituted with one or more oxo (C═O) or Rₖ groups; or R_{g1} and Rₕ₁ together with the nitrogen to which they are attached form a pyrrolidino, piperidino, piperazino, azetidino, morpholino, or thiomorpholino, wherein any pyrrolidino, piperidino, piperazino, azetidino, morpholino or thiomorpholino of R_{g1} and Rₕ₁ may be optionally substituted with one or more Rₖ or oxo groups;
R_{g} and Rₕ are each independently selected from H, (C₁-C₈)alkyl, (C₂-C₈)alkenyl, (C₂-C₈)alkynyl, (C₃-C₈)cycloalkyl, heterocycle, heteroaryl and aryl, wherein any aryl or heteroaryl of R_{g} or Rₕ may be optionally substituted with one or more Rₖ groups and wherein any alkyl, alkenyl, alkynyl, cycloalkyl or heterocycle of R_{g} or Rₕ may be optionally substituted with one or more oxo (C═O) or Rₖ groups; or R_{g} and Rₕ together with the nitrogen to which they are attached form a pyrrolidino, piperidino, piperazino, azetidino, morpholino, or thiomorpholino, wherein any pyrrolidino, piperidino, piperazino, azetidino, morpholino or thiomorpholino of R_{g} and Rₕ may be optionally substituted with one or more Rₖ or oxo groups;
each Rᵢ is independently H, (C₁-C₆)alkyl, (C₂-C₆)alkenyl, (C₂-C₆)alkynyl or (C₃-C₆)cycloalkyl;
each Rⱼ is independently selected from (C₁-C₆)alkyl, (C₂-C₆)alkenyl, (C₂-C₆)alkynyl, (C₃-C₆)cycloalkyl, heterocycle, heteroaryl and aryl, wherein any aryl or heteroaryl of Rⱼ may be optionally substituted with one or more Rₖ groups and wherein any alkyl, alkenyl, alkynyl, cycloalkyl or heterocycle of Rⱼ may be optionally substituted with one or more oxo (C═O) or Rₖ groups;
each Rₖ is independently selected from halo, R_{y}, CN, OH, —OR_{y}, —OC(O)R_{y}, —OC(O)NRᵥR_{w}, SH, —SR_{y}, —S(O)R_{y}, —S(O)₂OH, —S(O)₂R_{y}, —S(O)₂NRᵥR_{w}, —NRᵥR_{w}, —NRₓCOR_{y}, —NRₓCO₂R_{y}, —NRₓCONRᵥR_{w}, —NRₓS(O)₂R_{y}, —NRₓS(O)₂NRᵥR_{w}, NO₂, —C(O)Rᵤ, —C(O)ORᵤ and —C(O)NRᵥR_{w};
each Rₘ is independently H, (C₁-C₆)alkyl, (C₂-C₆)alkenyl, (C₂-C₆)alkynyl, (C₃-C₆)cycloalkyl, heterocycle, heteroaryl or aryl;
each Rₙ is independently (C₁-C₆)alkyl, (C₂-C₆)alkenyl, (C₂-C₆)alkynyl, (C₃-C₆)cycloalkyl, heterocycle, heteroaryl or aryl;
Rₒ and Rₚ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rₒ and Rₚ together with the nitrogen to which they are attached form a pyrrolidino, piperidino, piperazino, azetidino, morpholino, or thiomorpholino;
each R_{q} is independently (C₁-C₆)alkyl, (C₂-C₆)alkenyl, (C₂-C₆)alkynyl, (C₃-C₆)cycloalkyl, heterocycle, heteroaryl or aryl;
Rᵣ and Rₛ are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵣ and Rₛ together with the nitrogen to which they are attached form a pyrrolidino, piperidino, piperazino, azetidino, morpholino, or thiomorpholino;
each Rₜ is independently H, (C₁-C₆)alkyl, (C₂-C₆)alkenyl, (C₂-C₆) alkynyl or (C₃-C₆)cycloalkyl;
each Rᵤ is independently H, (C₁-C₆)alkyl, (C₂-C₆)alkenyl, (C₂-C₆)alkynyl, (C₃-C₆)cycloalkyl, heterocycle, heteroaryl or aryl;
Rᵥ and R_{w} are each independently selected from H, (C₁-C₆)alkyl, (C₂-C₆)alkenyl, (C₂-C₆)alkynyl, (C₃-C₆)cycloalkyl, heterocycle, heteroaryl and aryl; or Rᵥ and R_{w} together with the nitrogen to which they are attached form a pyrrolidino, piperidino, piperazino, azetidino, morpholino, or thiomorpholino, wherein any alkyl, alkenyl, alkynyl, cycloalkyl, heteroaryl, heterocycle, aryl pyrrolidino, piperidino, piperazino, azetidino, morpholino, or thiomorpholino of Rᵥ and R_{w} may be optionally substituted with one or more groups independently selected from CH₂OH, OH, NH₂ and CONH₂;
each Rₓ is independently H, (C₁-C₆)alkyl, (C₂-C₆)alkenyl, (C₂-C₆) alkynyl or (C₃-C₆)cycloalkyl;
each R_{y} is independently (C₁-C₆)alkyl, (C₂-C₆)alkenyl, (C₂-C₆)alkynyl, (C₃-C₆)cycloalkyl, heterocycle, heteroaryl or aryl, wherein any alkyl, alkenyl, alkynyl, cycloalkyl, heterocycle, heteroaryl or aryl of R_{y} may be optionally substituted with one or more groups selected from ORᵤ and NRᵥR_{w};
each R_{z} is independently halo, heteroaryl, (C₁-C₆)alkyl, CN, —O(C₁-C₆)alkyl, NO₂, —C(O)OH, —(C₁-C₆)alkylNH₂, —(C₁-C₆)alkylOH, —NHC(O)(C₁-C₆)alkyl or —NHC(O)(C₁-C₆)alkylCN, wherein heteroaryl is optionally substituted with —(C₁-C₆)alkylNH₂ or —(C₁-C₆)alkylOH;
each Rₐ₂ is independently H, (C₁-C₆)alkyl, (C₂-C₆)alkenyl, (C₂-C₆)alkynyl, (C₃-C₆)cycloalkyl, heterocycle, heteroaryl or aryl;
each R_{b2} is independently (C₁-C₆)alkyl, (C₂-C₆)alkenyl, (C₂-C₆)alkynyl, (C₃-C₆)cycloalkyl, heterocycle, heteroaryl or aryl;
R_{c2} and R_{d2} are each independently selected from H, (C₁-C₆)alkyl, (C₂-C₆)alkenyl, (C₂-C₆)alkynyl, (C₃-C₆)cycloalkyl, heterocycle, heteroaryl and aryl; or R_{c2} and R_{d2} together with the nitrogen to which they are attached form a pyrrolidino, piperidino, piperazino, azetidino, morpholino, or thiomorpholino; and
each Rₑ₂ is independently H, (C₁-C₆)alkyl, (C₂-C₆)alkenyl, (C₂-C₆) alkynyl or (C₃-C₆)cycloalkyl;
or a salt thereof.

12. The compound of claim 11 which is a compound of formula IIa: 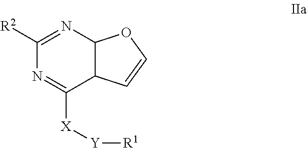
or a salt thereof.

13. The compound of claim 11 which is a compound of formula IIb: 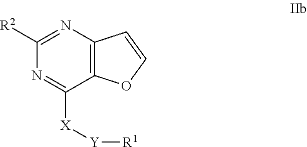
or a salt thereof.

14. The compound of claim 11 which is a compound of formula IIc: 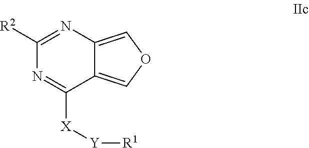
or a salt thereof.

15. The compound of claim 11 wherein R¹ is —C(O)NR_{g1}Rₕ₁, —NRᵢC(O)NR_{g1}Rₕ₁, —C(O)Rⱼ, or is absent.

16. (canceled)

17. The compound of claim 11 wherein R¹ is —C(O)NR_{g1}Rₕ₁.

18-22. (canceled)

23. The compound of claim 11 wherein —X—Y—R¹ is:

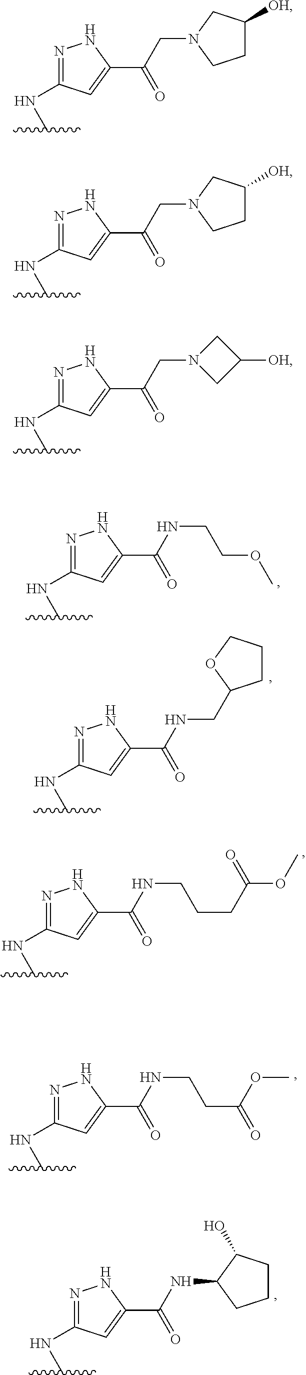

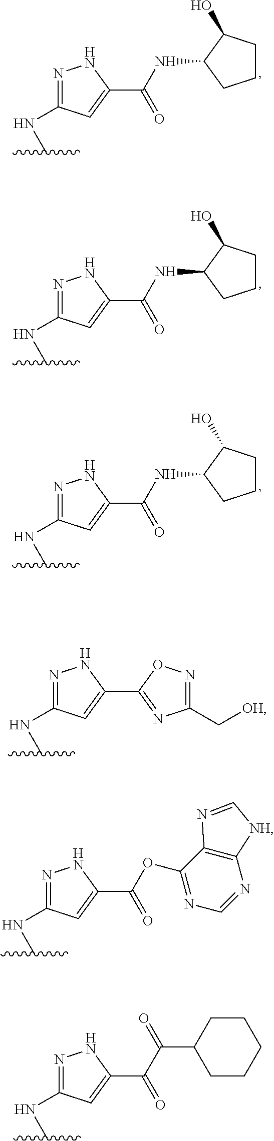

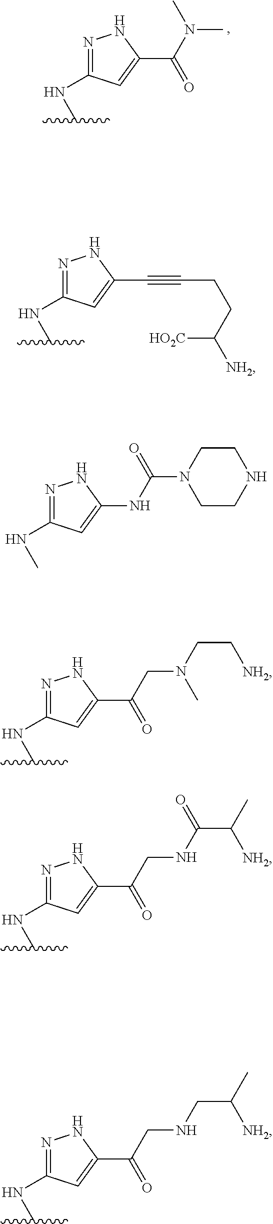

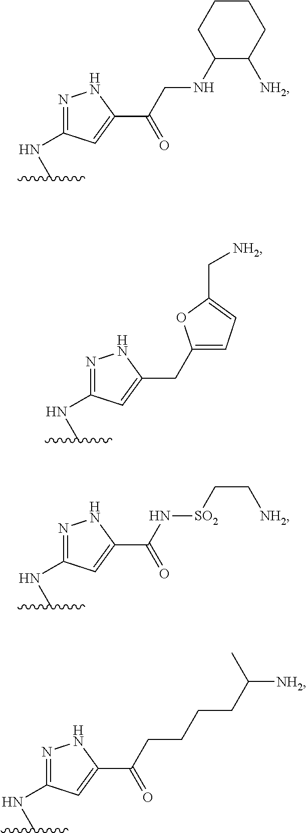

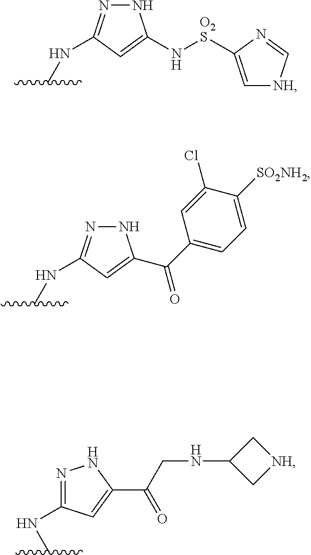

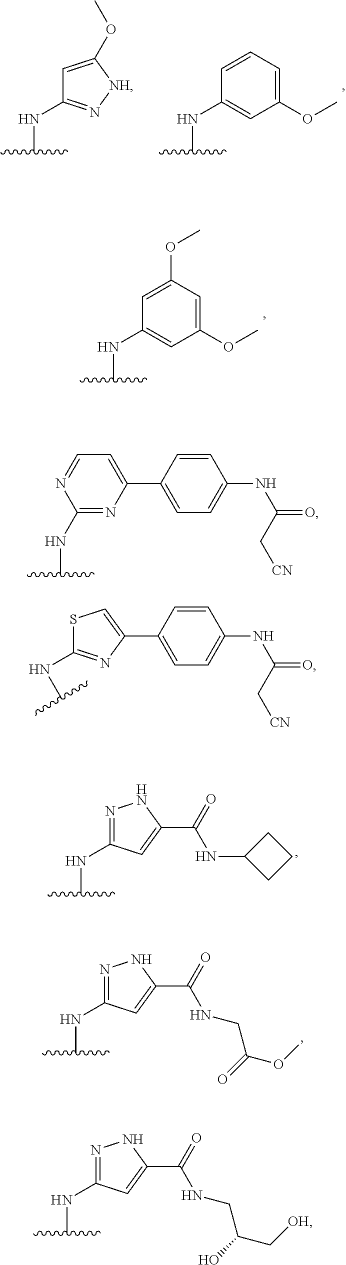

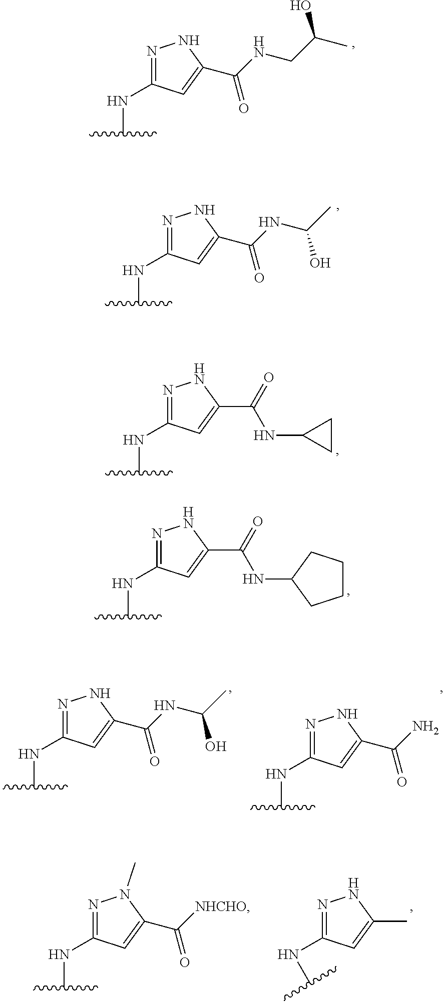

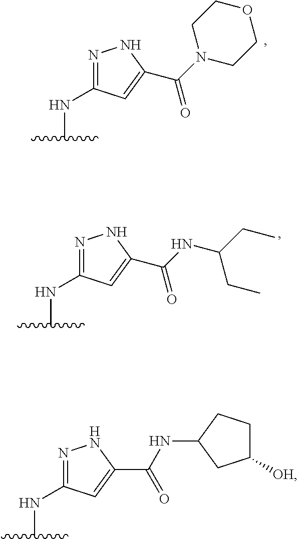

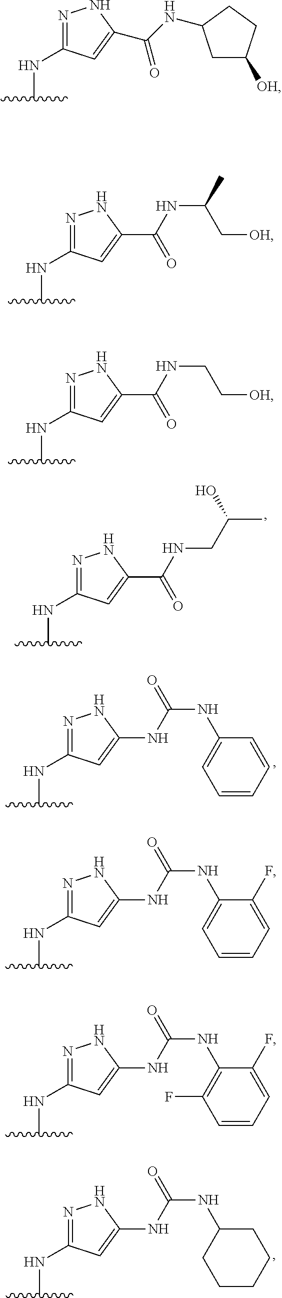

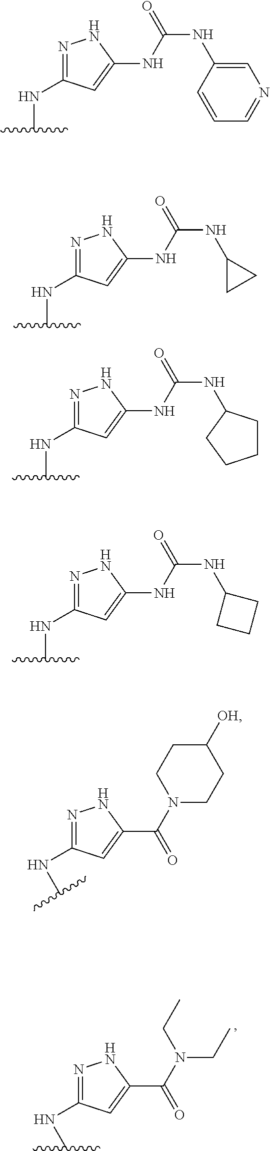

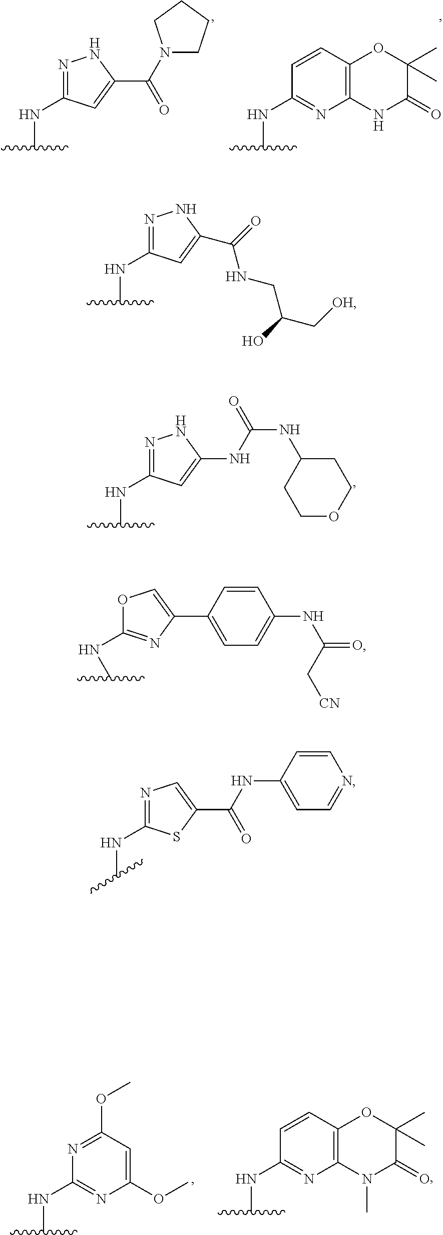

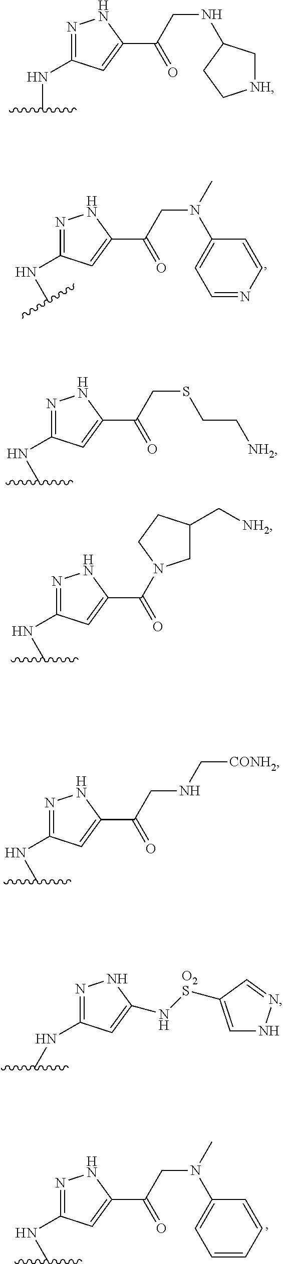

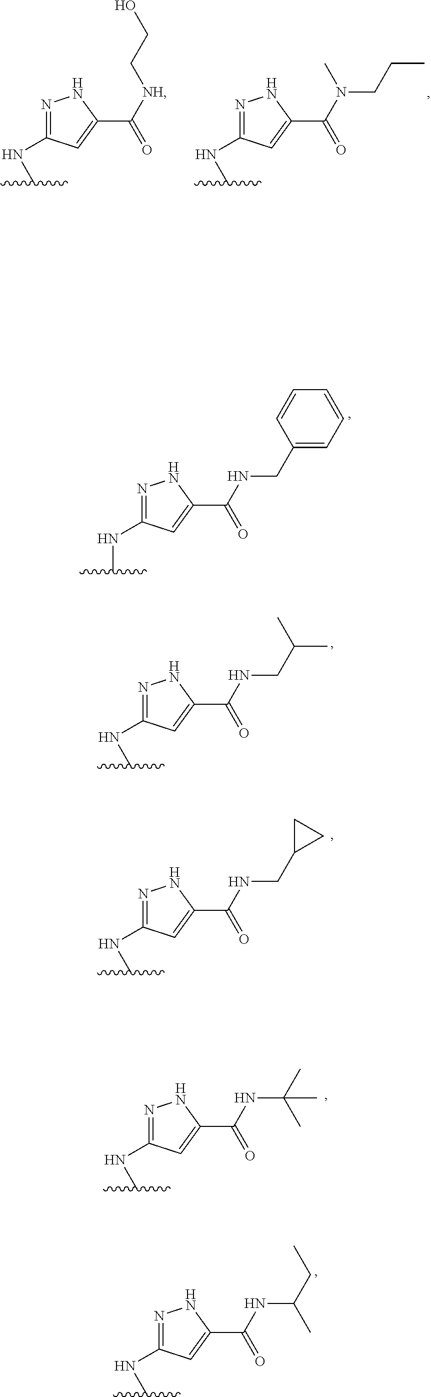

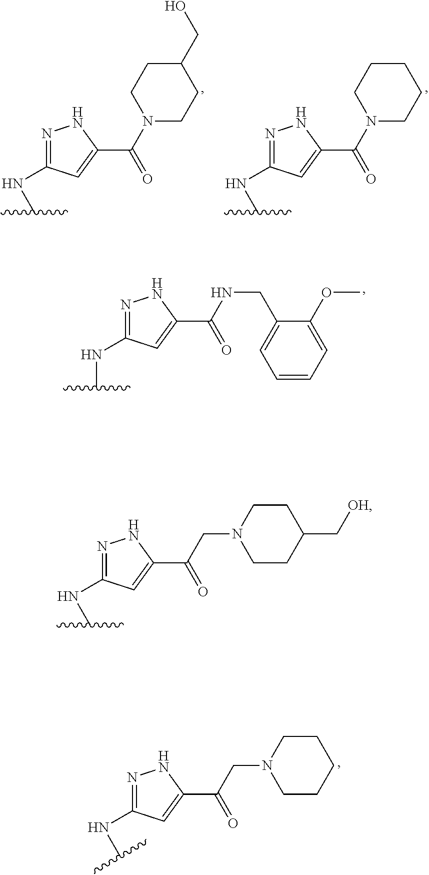

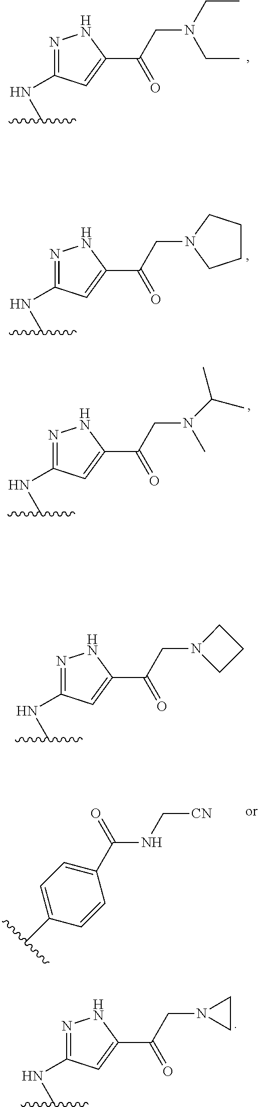

24. The compound of claim 1 wherein X is absent.

25. (canceled)

26. The compound of claim 1 wherein X is NH.

27. The compound of claim 1 wherein Y is heteroaryl, wherein any heteroaryl of Y may be optionally substituted with one or more Rₐ groups.

28. The compound of claim 1 wherein Y is pyrazolyl, pyrimidinyl, thiazolyl or oxazolyl, wherein any pyrazolyl, pyrimidinyl, thiazolyl or oxazolyl of Y may be optionally substituted with one or more Rₐ groups.

29. The compound of claim 1 wherein Y is

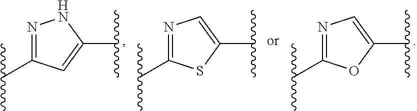

30. The compound of claim 1 wherein Y is

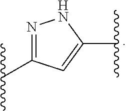

31. The compound of claim 1 wherein Y is aryl, wherein any aryl of Y may be optionally substituted with one or more Rₐ groups.

32. The compound of claim 1 wherein Y is phenyl.

33. The compound of claim 1 wherein R² is —NR⁶R⁷.

34. (canceled)

35. (canceled)

36. The compound of claim 33 wherein —NR⁶R⁷ is pyrrolidino, piperidino, piperazino, azetidino, morpholino, or thiomorpholino substituted with one or two R¹¹ groups.

37. The compound of claim 33 wherein —NR⁶R⁷ is pyrrolidino substituted with one or two R¹¹ groups.

38. The compound of claim 1 wherein R² is

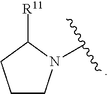

39. The compound of claim 1 wherein R¹¹ is selected from heteroaryl, aryl, —CH₂OH, —CH₂NH₂, —NHC(O)CH₃ and OH.

40-42. (canceled)

43. The compound of claim 1 wherein R² is:

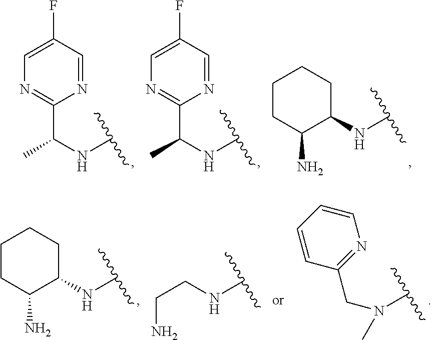

44. The compound of claim 1 wherein R² is:

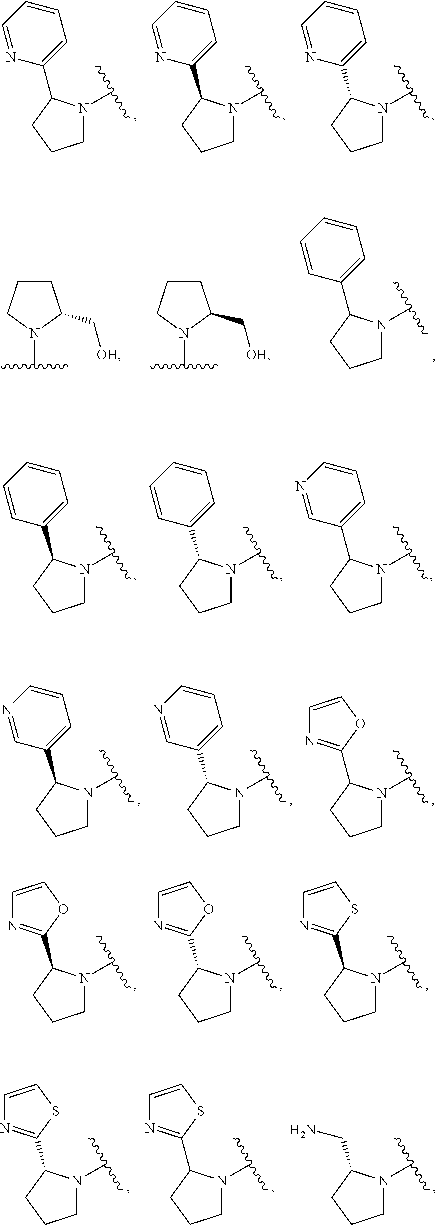

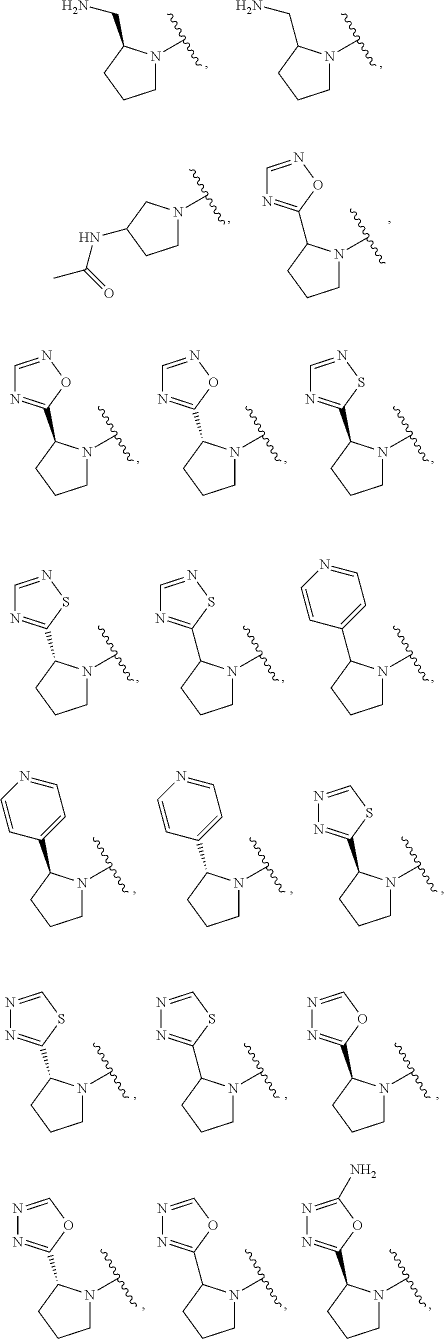

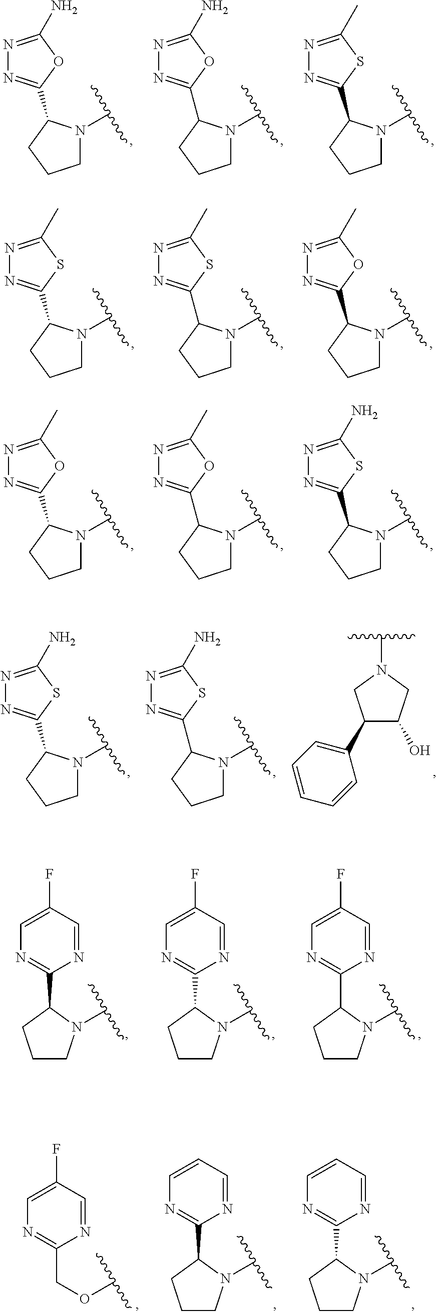

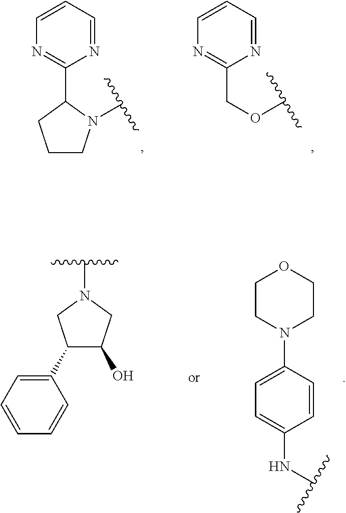

45. The compound of claim 1 which is:
N-(3-methoxyphenyl)-3-(2-(2-(pyridin-2-yl)pyrrolidin-1-yl)furo[3,2-d]pyrimidin-4-ylamino)-1H-pyrazole-5-carboxamide; or
3-(2-(2-(pyridin-2-yl)pyrrolidin-1-yl)furo[3,2-d]pyrimidin-4-ylamino)-N-(pyridin-4-yl)-1H-pyrazole-5-carboxamide;
or a salt thereof.

46. The compound of claim 11 which is:
N-cyclopropyl-5-(2-(2-(pyridin-2-yl)pyrrolidin-1-yl)furo[3,2-d]pyrimidin-4-ylamino)-1H-pyrazole-3-carboxamide;
N-cyclobutyl-5-(2-(2-(pyridin-2-yl)pyrrolidin-1-yl)furo[3,2-d]pyrimidin-4-ylamino)-1H-pyrazole-3-carboxamide (13E);
N-cyclobutyl-3-(2-(2-(pyridin-2-yl)pyrrolidin-1-yl)furo[3,2-d]pyrimidin-4-ylamino)-1H-pyrazole-5-carboxamide;
(S)—N-cyclopropyl-3-(2-(2-(hydroxymethyl)pyrrolidin-1-yl)furo[3,2-d]pyrimidin-4-ylamino)-1H-pyrazole-5-carboxamide; or
N-cyclobutyl-5-(2-(dimethylamino)furo[3,2-d]pyrimidin-4-ylamino)-1H-pyrazole-3-carboxamide;
or a salt thereof.

47. A pharmaceutical composition comprising a compound of formula I as described in claim 1, or a pharmaceutically acceptable salt thereof, in combination with a pharmaceutically acceptable diluent or carrier.

48. (canceled)

49. A method for treating a disease or condition associated with pathologic JAK activation in a mammal, comprising administering a compound of formula I as described in claim 1, or a pharmaceutically acceptable salt thereof, to the mammal.

50. (canceled)

51. (canceled)

52. The method of claim 49 wherein the disease or condition associated with pathologic JAK activation is cancer.

53. The method of claim 49 wherein the disease or condition associated with pathologic JAK activation is a hematologic or other malignancy.

54. A method for suppressing an immune response in a mammal, comprising administering a compound of formula I as described in claim 1, or a pharmaceutically acceptable salt thereof to the mammal.

55. (canceled)

56. (canceled)
